SEQUENCE LISTING (5 sequences)

SEQ ID NO: 1 - DNA, 69 bp, synthetic construct
  source 1..69: mol_type = other DNA
atccagagtgacgcagcaccgcccatgtcgaataggtgccacgtagggacttggacacggtggcttagt

SEQ ID NO: 2 - DNA, 77 bp, synthetic construct
  source 1..77: mol_type = other DNA
atccagagtgacgcagcacgagccttgtgtcgttcggagtgctggtccacgactactattggacacggtggcttagt

SEQ ID NO: 3 - DNA, 77 bp, synthetic construct
  source 1..77: mol_type = other DNA
gaggcataccagcttattcaaggggccggggcaaggggggggtaccgtggtaggacatagtaagtgcaatctgcgaa

SEQ ID NO: 4 - DNA, 67 bp, synthetic construct
  source 1..67: mol_type = other DNA
tgcgtgtgtagtgtgtctgggctacatcgatgagctgcctagggtccctcttagggatttgggcggg

SEQ ID NO: 5 - DNA, 66 bp, synthetic construct
  source 1..66: mol_type = other DNA
tgcgtgtgtactgtgtctggtcccgtagctactagcgaactccctgcctcttagggatttgggcgg

(19) United States
(12) Patent Application Publication
(10) Pub. No.: US 2025/0388907 A1
(43) Pub. Date: Dec. 25, 2025

(54) MULTISPECIFIC POLYNUCLEOTIDE NANOSTRUCTURES FOR CANCER DETECTION AND THERAPY

(71) Applicant: Atom Bioworks, Inc., Cary, NC (US)

(72) Inventors: Adam Friedman, Cary, NC (US); Sherwood Yao, Cary, NC (US)

(21) Appl. No.: 18/705,089

(22) PCT Filed: Oct. 28, 2022
(86) PCT No.: PCT/US2022/048248

§ 371 Date: Apr. 26, 2024

Related U.S. Application Data
(60) Provisional application No. 63/273,666, filed on Oct. 29, 2021.

(51) Int. Cl. C12N 15/115; A61K 31/7088

(52) U.S. Cl. CPC C12N 15/115; A61K 31/7088; C12N 2310/16

(57) ABSTRACT

Disclosed herein are multispecific polynucleotide nanostructures and techniques that use multispecific polynucleotide nanostructures as multimolecular recognition entities for the detection of antigens. For example, an artificial biopolymer complex can include a network of polynucleotides, a first set of binders attached at uniquely addressable loci on a first surface of the network of polynucleotides, and a second set of binders attached at uniquely addressable loci on a second surface of the network of polynucleotides. The first set of binders bind to antigens of a target analyte and the second set of binders bind to biomarkers of immune cells. The first and second set of binders are attached at the uniquely addressable loci on the network of polynucleotides separated by predetermined inter-binder and/or intra-binder distances that match intermolecular spacing and/or intramolecular spacing of antigens on a surface of the target analyte and biomarkers on a surface of the immune cells, respectively.

### RELATED APPLICATION

The application claims priority to U.S. Provisional Application No. 63/273,666, filed on Oct. 29, 2021. The entire disclosure of said provisional application is herein incorporated by reference for all purposes.

### FIELD

The present disclosure relates to antigen detection and therapeutic targeting, and in particular to multispecific polynucleotide nanostructures and techniques that use multispecific polynucleotide nanostructures as multimolecular recognition entities for the detection of antigens and therapy thereof.

### BACKGROUND

In the battle against cancer, early detection and the eradication of cells resistant to —or inaccessible by—existing therapeutics are key factors for successful treatment. However, the detection of cancer in the early stage has been hindered by the intrinsic limits of conventional cancer diagnostic methods. Nanotechnology provides new molecular detection agents and materials and has therefore been investigated for the detection of cancer biomarkers and cancer cells, as well as for in vivo imaging. Although nanotechnology has not yet been deployed clinically for cancer diagnosis, it is already on the market in a variety of medical tests and screens, such as the use of gold nanoparticles in home pregnancy tests. For cancer diagnosis, nanotechnology is being investigated for the capture of cancer biomarkers, such as cancer-associated proteins, circulating tumor DNA, circulating tumor cells, and exosomes. In therapy, the ability of targeted therapeutics to eradicate phenotypically heterogeneous cancer cell populations within patients via, for example, the targeting of two biomarkers simultaneously, has made progress over the past few years with the development of antibodies and CAR-T cells, but there is a lack of therapeutics capable of targeting more than two biomarkers on one therapeutic molecular entity, and that can do so with limited to no cytotoxicity.

A cancer biomarker acts as a measurable biological molecule that can be found in blood and other tissues or body fluids, such as saliva and urine, indicating that cancer exists in the body. Cancer biomarkers may be proteins (secreted proteins or cell surface proteins), carbohydrates, or nucleic acids (circulating tumor DNA, miRNA, etc.) that are secreted by the body or cancer cells when cancer is present. The measurement of certain cancer biomarker levels enables early detection of cancer or tumor recurrence and helps monitor the efficacy of the therapy. Nevertheless, the use of biomarkers has been limited by several barriers, including low biomarker concentrations in body fluids, heterogeneity in the abundance and timing of biomarkers within patients, and the difficulty in carrying out prospective studies. Nanotechnology provides high sensitivity, specificity, and multiplexed measurement capacity, and thus the ability to overcome some of these barriers.

Another key factor for the successful treatment of cancer, is the therapeutic options available for the patient. Cancer therapies are typically limited to surgery, radiation, and chemotherapy. All three methods risk damage to normal tissues, the inducement of therapeutic resistance, or incomplete eradication of cancer. Nanotechnology offers the means to target chemotherapies directly and selectively to cancerous cells and neoplasms, guide in surgical resection of tumors, and enhance the therapeutic efficacy of radiation-based and other current treatment modalities. All of this can add up to a decreased risk to the patient, an increased probability of survival, and overall improved patient outcomes.

Nanotechnology is being investigated for cancer therapy that extends beyond drug delivery into the creation of new therapeutics available only through the use of nanomaterial properties. Although small compared to cells, nanoparticles are large enough to encapsulate many small molecule compounds, which can be of multiple types, and avoid some in vivo clearance pathways by being above the glomerular filtration rate cut-off. At the same time, the relatively large surface area of a nanoparticle can be functionalized with binders, including small molecules, DNA or RNA strands, peptides, aptamers, or antibodies. These binders can be used for therapeutic effects or to direct nanoparticle fate in vivo. These properties enable combination drug delivery, multi-modality treatment, and combined therapeutic and diagnostic, known as “theranostic,” action. The physical properties of nanoparticles, such as energy absorption and re-radiation, can also be used to disrupt diseased tissue, as in laser ablation and hyperthermia applications.

### SUMMARY

Provided herein, according to various embodiments, is an artificial biopolymer complex comprising: a network of polynucleotides comprising structural units connected to one another via a series of arms and junctions, where intersections of two or more (e.g., three or more) arms form the junctions; a first set of binders attached to a first surface of the network of polynucleotides, where: the first set of binders bind to antigens of a target analyte; and the first set of binders are attached at uniquely addressable loci on the arms forming the junctions, where the uniquely addressable loci are separated by predetermined inter-binder distances such that the first set of binders are positioned on the network of polynucleotides in a predetermined two-dimensional or three-dimensional spatial pattern that matches a two-dimensional or three-dimensional spatial pattern of the antigens on the target analyte; and a second set of binders attached to a second surface of the network of polynucleotides, where: the second set of binders bind to biomarkers of the immune cells; and the second set of binders are attached at uniquely addressable loci on the arms forming the junctions, where the uniquely addressable loci are separated by predetermined inter-binder distances such that the second set of binders are positioned on the network of polynucleotides in a predetermined two-dimensional or three-dimensional spatial pattern that matches a two-dimensional or three-dimensional spatial pattern of the biomarkers on the immune cells.

Also provided herein is an artificial biopolymer complex comprising: a network of polynucleotides comprising structural units connected to one another via a series of arms and junctions, wherein intersections of two or more arms (e.g., three or more arms) form the junctions; antigen binders attached to a surface of the network of polynucleotides, wherein: the antigen binders bind to antigens of a target analyte; and wherein at least some of the antigen binders are attached at uniquely addressable loci on the arms forming the junctions, wherein the uniquely addressable loci are separated by predetermined inter-binder distances such that the antigen binders are positioned on the network of polynucleotides in a predetermined two-dimensional or three-dimensional spatial pattern that matches a two-dimensional or three-dimensional spatial pattern of the antigens on the target analyte.

In some embodiments, each of the structural units have a predetermined shape defined by one or more strands of polynucleotides; at least a portion of the one or more strands of polynucleotides of each structural unit is complementary to at least a portion of the one or more strands of polynucleotides of another structural unit, and the complementary portions of the strands of the polynucleotides are hybridized to connect the structural units; the complementary portions of the strands of the polynucleotides form the arms with a predetermined length, and the intersections of the two or more arms form the junctions at a predetermined distance from one another based on the predetermined length of the arms.

Also provided herein is an artificial biopolymer complex comprising: a network of polynucleotides comprising connector chains of polynucleotides attached to a structural chain of polynucleotides, wherein the connector chains are shorter than the structural chain; antigen binders attached to a first surface of the network of polynucleotides, wherein: the antigen binders bind to antigens of a target analyte; and the antigen binders are attached at uniquely addressable loci on the structural chain via the connector chains, wherein the uniquely addressable loci are separated by predetermined inter-binder distances such that the antigen binders are positioned on the network of polynucleotides in a predetermined two-dimensional or three-dimensional spatial pattern that matches a two-dimensional or three-dimensional spatial pattern of the antigens on the target analyte.

In some embodiments, the artificial biopolymer complex further comprises one or more therapeutic agents covalently or non-covalently attached to the network of polynucleotides.

In some embodiments, the two-dimensional or three-dimensional spatial pattern of the antigens is defined by intermolecular spacing of the antigens on a surface of the target analyte.

In some embodiments, the predetermined inter-binder distances of the uniquely addressable loci of the first set of binders match the intermolecular spacing of the antigens such that the first set of binders on the first surface of the network of polynucleotides align spatially with the antigens on the surface of the target analyte.

In some embodiments, each of the antigens is (i) a length and width in angstroms or nanometers (e.g., 1 nm-999 nm, or 1-800 nm, 1-600 nm, 1-500 nm, 1-400 nm, 1-300 nm, 1-200 nm, 2-150 nm, 2-100 nm, 2-90 nm, or 4-50 nm) from other antigens on the target analyte or (ii) a length, width, and depth from the other antigens on the target analyte, which define the intermolecular spacing of the antigens; and each of the binders of the first set of binders is (i) a length and width in angstroms or nanometers from other binders of the first set of binders on the network of polynucleotides or (ii) a length, width, and depth in angstroms or nanometers from the other binders of the first set of binders on the network of polynucleotides, which defines the predetermined inter-binder distances of the uniquely addressable loci of the first set of binders; and the predetermined inter-binder distances of the uniquely addressable loci of the first set of binders match the intermolecular spacing of the antigens such that the first set of binders on the first surface of the network of polynucleotides align spatially with the antigens on the surface of the target analyte.

In some embodiments, the two-dimensional or three-dimensional spatial pattern of the biomarkers is defined by intermolecular spacing of the biomarkers on a surface of the immune cells.

In some embodiments, the predetermined inter-binder distances of the uniquely addressable loci of the second set of binders match the intermolecular spacing of the biomarkers such that the second set of binders on the second surface of the network of polynucleotides align spatially with the biomarkers on the surface of the immune cells.

In some embodiments, each of the biomarkers is (i) a length and width in angstroms or nanometers from other biomarkers on the immune cells or (ii) a length, width, and depth from the other biomarkers on the immune cells, which define the intermolecular spacing of the biomarkers; and each of the binders of the second set of binders is (i) a length and width in angstroms or nanometers from other binders of the second set of binders on the network of polynucleotides or (ii) a length, width, and depth in angstroms or nanometers from the other binders of the second set of binders on the network of polynucleotides, which defines the predetermined inter-binder distances of the uniquely addressable loci of the second set of binders; and the predetermined inter-binder distances of the uniquely addressable loci of the second set of binders match the intermolecular spacing of the biomarkers such that the second set of binders on the second surface of the network of polynucleotides align spatially with the biomarkers on the surface of the immune cells.

In some embodiments, each of the antigens comprises one or more epitopes or domains; the first set of binders is arranged in sets of clustered antigen binders; each binder of a set of clustered antigen binders is attached to one of the two or more arms that form a junction; and the binders of each of the sets of clustered antigen binders are attached to the arms at uniquely addressable loci that are a predetermined distance from the junction, where the uniquely addressable loci are separated by predetermined intra-binder distances such that each set of clustered antigen binders is positioned on the network of polynucleotides in a predetermined two-dimensional or three-dimensional spatial pattern that matches a two-dimensional or three-dimensional spatial pattern of the one or more epitopes on an antigen.

In some embodiments, the two-dimensional or three-dimensional spatial pattern of the one or more epitopes is defined by intramolecular spacing of the one or more epitopes on a surface of the antigen.

In some embodiments, the predetermined intra-binder distances of the uniquely addressable loci of the binders of each of the sets of clustered antigen binders match the intramolecular spacing of the one or more epitopes such that the sets of clustered antigen binders on the first surface of the network of polynucleotides align spatially with the epitopes on the surface of the antigens.

In some embodiments, each of the one or more epitopes is (i) a length and width in angstroms or nanometers from other epitopes of the one or more epitopes on the antigen or (ii) a length, width, and depth from the other epitopes of the one or more epitopes on the antigen, which define the intramolecular spacing of the one or more epitopes or domains; and each of the binders of each of the sets of clustered antigen binders is (i) a length and width in angstroms or nanometers from other binders of each of the sets of clustered antigen binders on the network of polynucleotides or (ii) a length, width, and depth in angstroms or nanometers from the other binders of each of the sets of clustered antigen binders on the network of polynucleotides, which defines the predetermined intra-binder distances of the uniquely addressable loci of the binders of each of the sets of clustered antigen binders; and the predetermined intra-binder distances of the uniquely addressable loci of the binders of each of the sets of clustered antigen binders match the intermolecular spacing of the epitopes such that each of the sets of clustered antigen binders on the first surface of the network of polynucleotides align spatially with the epitopes on the surface of the antigens.

In some embodiments, the predetermined intra-binder distances of the uniquely addressable loci of the binders of each of the sets of clustered antigen binders is between 1 nm and 15 nm.

In some embodiments, each of the biomarkers comprises one or more epitopes or domains; the second set of binders are arranged in sets of clustered biomarker binders; each binder of a set of clustered biomarker binders is attached to one of the two or more arms that form a junction; and the binders of each of the sets of clustered biomarker binders are attached to the arms at uniquely addressable loci that are a predetermined distance from the junction, where the uniquely addressable loci are separated by predetermined intra-binder distances such that each set of clustered biomarker binders is positioned on the network of polynucleotides in a predetermined two-dimensional or three-dimensional spatial pattern that matches a two-dimensional or three-dimensional spatial pattern of the one or more epitopes on a biomarker.

In some embodiments, the two-dimensional or three-dimensional spatial pattern of the one or more epitopes is defined by intramolecular spacing of the one or more epitopes on a surface of the biomarker.

In some embodiments, the predetermined intra-binder distances of the uniquely addressable loci of the binders of each of the sets of clustered biomarker binders match the intramolecular spacing of the one or more epitopes such that the sets of clustered biomarker binders on the second surface of the network of polynucleotides align spatially with the epitopes on the surface of the biomarkers.

In some embodiments, each of the one or more epitopes is (i) a length and width in angstroms or nanometers from other epitopes of the one or more epitopes on the biomarker or (ii) a length, width, and depth from the other epitopes of the one or more epitopes on the biomarker, which define the intramolecular spacing of the one or more epitopes or domains; and each of the binders of each of the sets of clustered biomarker binders is (i) a length and width in angstroms or nanometers from other binders of each of the sets of clustered biomarker binders on the network of polynucleotides or (ii) a length, width, and depth in angstroms or nanometers from the other binders of each of the sets of clustered biomarker binders on the network of polynucleotides, which defines the predetermined intra-binder distances of the uniquely addressable loci of the binders of each of the sets of clustered biomarker binders; and the predetermined inter-binder distances of the uniquely addressable loci of the binders of each of the sets of clustered biomarker binders match the intermolecular spacing of the epitopes such that each of the sets of clustered biomarker binders on the second surface of the network of polynucleotides align spatially with the epitopes on the surface of the biomarkers.

In some embodiments, the predetermined intra-binder distances of the uniquely addressable loci of the binders of each of the sets of clustered biomarker binders is between 1 nm and 15 nm.

In some embodiments, the artificial biopolymer complex further comprises one or more therapeutic agents attached to the network of polynucleotides.

In some embodiments, the network of polynucleotides has a curvature such that the second set of binders is at least partially enveloped by the second surface of the network of polynucleotides.

In some embodiments, some or all of the polynucleotides in the network of polynucleotides has been cross-linked to stabilize the structure and increase a half-life of a therapeutic agent.

In some embodiments, the network of polynucleotides has been modified with polymers, peptides, proteins, lipids, or a combination hereof to modulate pharmacokinetics and distribution in vivo. For example, these polymers, peptides, proteins, lipids, or a combination hereof can be attached to the network of polynucleotides covalently or noncovalently.

Also provided herein, according to various embodiments, is an artificial biopolymer complex comprising: a network of polynucleotides comprising connector chains of polynucleotides attached to a structural chain of polynucleotides, where the connector chains are shorter than the structural chain; a first set of binders attached to a first surface of the network of polynucleotides, where: the first set of binders bind to antigens of a target analyte; and the first set of binders are attached at uniquely addressable loci on the structural chain via the connector chains, where the uniquely addressable loci are separated by predetermined inter-binder distances such that the first set of binders are positioned on the network of polynucleotides in a predetermined two-dimensional or three-dimensional spatial pattern that matches a two-dimensional or three-dimensional spatial pattern of the antigens on the target analyte; and a second set of binders attached to a second surface of the network of polynucleotides, where: the second set of binders bind to biomarkers on immune cells; and the second set of binders are attached at uniquely addressable loci on the structural chain via the connector chains, where the uniquely addressable loci are separated by predetermined inter-binder distances such that the second set of binders are positioned on the network of polynucleotides in a predetermined two-dimensional or three-dimensional spatial pattern that matches a two-dimensional or three-dimensional spatial pattern of the biomarkers on the immune cells.

### Terminology

As used herein, the term “target analyte” refers to any target the artificial biopolymer complex can bind. In some embodiments, it refers to one or more cells in a subject who suffer from a disease condition, for example, one or more tumor cells in a tumor patient.

As used herein, the term “inter-binder distances” refers to the distances between adjacent uniquely addressable loci of binders on the network of polynucleotides.

As used herein, the term “intra-binder distances” refers to the inter-binder distances between adjacent uniquely addressable loci of binders on the network of polynucleotides that bind to the different epitopes of the same antigen.

As used herein, the term “inter-cluster distances” refers the distances between the centers of adjacent clusters of uniquely addressable loci of binders on the network of polynucleotides

As used herein, the term “intra-cluster distances” refers to inter-binder distances between adjacent uniquely addressable loci of binders within one cluster of uniquely addressable loci of binders on the network of polynucleotides.

As used herein, the term “intermolecular spacing” refers to the spacing between two adjacent antigens on a target analyte.

As used herein, the term “intramolecular spacing” refers to the spacing between two adjacent epitopes of an antigen comprising two or more epitopes on a target analyte. FIG. 25D shows one example where an antigen contains three different epitopes (represented by solid spheres) which can be recognized by three different binders on a biopolymer. In some cases, the two or more epitopes are different. In some cases, the two or more epitopes are the same. In some instances, an antigen is a multimeric antigen (for example, the spike protein that forms a trimer on target cells), each monomer comprising an epitope recognizable by an antigen binder, the intramolecular spacing is essentially the spacing between the adjacent monomers of the antigen.

As used herein, the term “dock” and the term “display” are used interchangeably to mean attaching binders to the network of polynucleotides. In some embodiments, docking a binder to the network of polynucleotides is through hybridization between a polynucleotide sequence in the binder (or a polynucleotide conjugated to the binder) and an anchor sequence (e.g., a sequence in the arms or the connector chains) on the network of polynucleotides.

In some embodiments, at least a portion of each chain of the connector chains is complementary to at least a portion of the structural chain, and the complementary portions of the connector chains and structural chain are hybridized to connect the connector chains to the structural chain.

In some embodiments, the two-dimensional or three-dimensional spatial pattern of the antigens is defined by intermolecular spacing of the antigens on a surface of the target analyte.

In some embodiments, the predetermined inter-binder distances of the uniquely addressable loci of the first set of binders match the intermolecular spacing of the antigens such that the first set of binders on the first surface of the network of polynucleotides aligns spatially with the antigens on the surface of the target analyte.

In some embodiments, each of the antigens is (i) a length and width in angstroms or nanometers from other antigens on the target analyte or (ii) a length, width, and depth from the other antigens on the target analyte, which define the intermolecular spacing of the antigens; and each of the binders of the first set of binders is (i) a length and width in angstroms or nanometers from other binders of the first set of binders on the network of polynucleotides or (ii) a length, width, and depth in angstroms or nanometers from the other binders of the first set of binders on the network of polynucleotides, which defines the predetermined inter-binder distances of the uniquely addressable loci of the first set of binders; and the predetermined inter-binder distances of the uniquely addressable loci of the first set of binders match the intermolecular spacing of the antigens such that the first set of binders on the first surface of the network of polynucleotides align spatially with the antigens on the surface of the target analyte.

In some embodiments, the two-dimensional or three-dimensional spatial pattern of the biomarkers is defined by intermolecular spacing of the biomarkers on a surface of the immune cells.

In some embodiments, the predetermined inter-binder distances of the uniquely addressable loci of the second set of binders match the intermolecular spacing of the biomarkers such that the second set of binders on the second surface of the network of polynucleotides align spatially with the biomarkers on the surface of the immune cells.

In some embodiments, each of the biomarkers is (i) a length and width in angstroms or nanometers from other biomarkers on the immune cells or (ii) a length, width, and depth from the other biomarkers on the immune cells, which define the intermolecular spacing of the biomarkers; and each of the binders of the second set of binders is (i) a length and width in angstroms or nanometers from other binders of the second set of binders on the network of polynucleotides or (ii) a length, width, and depth in angstroms or nanometers from the other binders of the second set of binders on the network of polynucleotides, which defines the predetermined inter-binder distances of the uniquely addressable loci of the second set of binders; and the predetermined inter-binder distances of the uniquely addressable loci of the second set of binders match the intermolecular spacing of the biomarkers such that the second set of binders on the second surface of the network of polynucleotides align spatially with the biomarkers on the surface of the immune cells.

In some embodiments, each of the antigens comprises one or more epitopes or domains; the first set of binders are arranged in sets of clustered antigen binders; and the binders of each of the sets of clustered antigen binders are attached at uniquely addressable loci on the structural chain via the connector chains, where the uniquely addressable loci are separated by predetermined intra-binder distances such that each set of clustered antigen binders is positioned on the network of polynucleotides in a predetermined two-dimensional or three-dimensional spatial pattern that matches a two-dimensional or three-dimensional spatial pattern of the one or more epitopes on an antigen.

In some embodiments, the two-dimensional or three-dimensional spatial pattern of the one or more epitopes is defined by intramolecular spacing of the one or more epitopes on a surface of the antigen.

In some embodiments, the predetermined intra-binder distances of the uniquely addressable loci of the binders of each of the sets of clustered antigen binders match the intramolecular spacing of the one or more epitopes such that the sets of clustered antigen binders on the first surface of the network of polynucleotides align spatially with the epitopes on the surface of the antigens.

In some embodiments, each of the one or more epitopes is (i) a length and width in angstroms or nanometers from other epitopes of the one or more epitopes on the antigen or (ii) a length, width, and depth from the other epitopes of the one or more epitopes on the antigen, which define the intramolecular spacing of the one or more epitopes or domains; and each of the binders of each of the sets of clustered antigen binders is (i) a length and width in angstroms or nanometers from other binders of each of the sets of clustered antigen binders on the network of polynucleotides or (ii) a length, width, and depth in angstroms or nanometers from the other binders of each of the sets of clustered antigen binders on the network of polynucleotides, which defines the predetermined intra-binder distances of the uniquely addressable loci of the binders of each of the sets of clustered antigen binders; and the predetermined intra-binder distances of the uniquely addressable loci of the binders of each of the sets of clustered antigen binders match the intermolecular spacing of the epitopes such that each of the sets of clustered antigen binders on the first surface of the network of polynucleotides align spatially with the epitopes on the surface of the antigens.

In some embodiments, the predetermined intra-binder distances of the uniquely addressable loci of the binders of each antigen binder cluster are between 1 nm and 15 nm.

In some embodiments, each of the biomarkers comprises one or more epitopes or domains; the second set of binders is arranged in sets of clustered biomarker binders; and the binders of each of the sets of clustered biomarker binders are attached at uniquely addressable loci on the structural chain via the connector chains, where the uniquely addressable loci are separated by predetermined intra-binder distances such that each set of clustered biomarker binders is positioned on the network of polynucleotides in a predetermined two-dimensional or three-dimensional spatial pattern that matches a two-dimensional or three-dimensional spatial pattern of the one or more epitopes on a biomarker.

In some embodiments, the two-dimensional or three-dimensional spatial pattern of the one or more epitopes is defined by intramolecular spacing of the one or more epitopes on a surface of the biomarker.

In some embodiments, the predetermined intra-binder distances of the uniquely addressable loci of the binders of each of the sets of clustered biomarker binders match the intramolecular spacing of the one or more epitopes such that the sets of clustered biomarker binders on the second surface of the network of polynucleotides align spatially with the epitopes on the surface of the biomarkers.

In some embodiments, each of the one or more epitopes is (i) a length and width in angstroms or nanometers from other epitopes of the one or more epitopes on the biomarker or (ii) a length, width, and depth from the other epitopes of the one or more epitopes on the biomarker, which define the intramolecular spacing of the one or more epitopes or domains; and each of the binders of each of the sets of clustered biomarker binders is (i) a length and width in angstroms or nanometers from other binders of each of the sets of clustered biomarker binders on the network of polynucleotides or (ii) a length, width, and depth in angstroms or nanometers from the other binders of each of the sets of clustered biomarker binders on the network of polynucleotides, which defines the predetermined intra-binder distances of the uniquely addressable loci of the binders of each of the sets of clustered biomarker binders; and the predetermined inter-binder distances of the uniquely addressable loci of the binders of each of the sets of clustered biomarker binders match the intermolecular spacing of the epitopes such that each of the sets of clustered biomarker binders on the second surface of the network of polynucleotides align spatially with the epitopes on the surface of the biomarkers.

In some embodiments, the predetermined intra-binder distances of the uniquely addressable loci of the binders of each of the sets of clustered biomarker binders are between 1 nm and 15 nm.

In some embodiments, the artificial biopolymer complex further comprises one or more therapeutic agents attached to the network of polynucleotides.

In some embodiments, the network of polynucleotides has a curvature such that the second set of binders is at least partially enveloped by the second surface of the network of polynucleotides.

In some embodiments, the network of polynucleotides has been cross-linked to stabilize the structure and increase a half-life of a therapeutic agent.

In some embodiments, the network of polynucleotides has been modified with polymers, peptides, proteins, lipids, or a combination hereof to modulate pharmacokinetics and distribution in vivo.

Also provided herein, according to various embodiments, is a method for treating a subject, the method comprising: obtaining the artificial biopolymer complex of any embodiment or combination thereof described herein; and administering the artificial biopolymer complex to the subject in an amount sufficient to provide a treatment effect.

In some embodiments, the treatment effect is a prophylactic effect or a therapeutic effect.

In some embodiments, the treatment effect is facilitated by binding of the first set of binders to the antigens and the second set of binders to the biomarkers.

In some embodiments, the binding of the second set of binders to the biomarkers activates an immune response by the immune cells.

In some embodiments, the binding of the first set of binders to the antigens activates a response by the target analyte to the artificial biopolymer complex.

In some embodiments, the binding of the first set of binders to the antigens labels a target analyte for detection, quantification, and/or treatment.

In some embodiments, the response is a cellular internalization of the artificial biopolymer complex by the target analyte.

In some embodiments, the cellular internalization of the artificial biopolymer complex by the target analyte causes the release of the one or more therapeutic agents from the network of polynucleotides.

In some embodiments, the binding of the first set of binders to the antigens causes the curvature of the network of polynucleotides to flatten, which facilitates the binding of the second set of binders to the biomarkers.

In some embodiments, the binding of the first set of binders to the antigens causes the network of polynucleotides to curve, which blocks or facilitates the binding of the second set of binders to the biomarkers.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead placed upon illustrating the principles of various embodiments of the invention.

FIG. 1 illustrates an artificial biopolymer complex that includes multispecific polynucleotide nanostructures as multimolecular recognition entities for the detection of antigens according to various embodiments of the present disclosure.

FIG. 2 illustrates a network of polynucleotides that provide a structure for a defined spacing of binders according to various embodiments of the present disclosure.

FIG. 3 illustrates the intra-binder, inter-binder, intra-cluster, and inter-cluster binder distances according to various embodiments of the present disclosure.

FIG. 4 illustrates the intermolecular and intramolecular spacing of antigens and epitopes according to various embodiments of the present disclosure.

FIG. 5 illustrates an alternative artificial biopolymer complex that includes multispecific polynucleotide nanostructures as multimolecular recognition entities for detecting antigens according to various embodiments of the present disclosure.

FIG. 6 illustrates an alternative network of polynucleotides that provide a structure for a defined spacing of binders according to various embodiments of the present disclosure.

FIG. 7 illustrates intra-binder, inter-binder, intra-cluster, and inter-cluster binder distances and the intermolecular and intramolecular spacing of antigens and epitopes according to various embodiments of the present disclosure.

FIG. 8 illustrates aptamer attachment to a multispecific polynucleotide nanostructure according to various embodiments of the present disclosure.

FIG. 9 illustrates one or more therapeutic agents attached to the multispecific polynucleotide nanostructure according to various embodiments of the present disclosure.

FIG. 10 illustrates a process for treating a subject using an artificial biopolymer complex according to various embodiments of the present disclosure.

FIG. 11A-11E show results of flow cytometry analysis demonstrating binding of aptamers (both selected by Atom Bioworks and taken from the literature) to endogenously expressed targets on Human acute myeloid leukemia (AML) cell lines KASUMI-1, MM1S, HL-60, and HEL92.1.7.

FIG. 12A shows the results of flow cytometry analysis and quantifies the dissociation constants between the aptamers and the human AML cells. FIG. 12B shows the K_{D} determinations of ABWT050902 aptamer alone and ABWT050902 immobilized on the TRD1 (both conditions binding to CD117). A) Binding of ABWT050902 alone to CD117 across concentrations; K_{D}=1220 nM. B) Binding of ABWT050902 immobilized on TRD1 to CD117 across concentrations; K_{D}=0.47 nM.

FIG. 13 shows the results of the killing of AML cells by aptamers loaded with daunorubicin. “Untreated” cells are negative killing controls. “10% DMSO” and “40 μM Daunorubicin” are positive killing controls. Negative control cells MM1S were only treated with aptamers at 4 μM and 2 μM. Aptamers did not increase the daunorubicin sensitivity of MM1S above 40 μM daunorubicin alone. Only aptamers carrying daunorubicin and binding KASUMI-1 cells increased killing.

FIG. 14A shows the results of the killing of AML cells by aptamers loaded with daunorubicin, free and immobilized on networks of polynucleotides. “Untreated” cells are negative killing controls. “10% DMSO” and “0.4 μM Daunorubicin” are positive killing controls. 0.4 μM Daunorubicin is the amount of daunorubicin intercalated into aptamers across all conditions. “3x3v1 net (Empty)” is 3x3v1 net without aptamers (therefore, no daunorubicin). “TRD1 (Empty)” is TRD1 without aptamers (therefore, no daunorubicin). “Scramble Aptamer 1”, “Scramble Aptamer 2”, and “Scramble Aptamer 3” are three oligos with lengths and A:T:C:G ratios similar to the tested aptamers but whose nucleotide sequences are randomized but also loaded with daunorubicin. Across all the conditions except for the positive cell killing condition “10% DMSO”, almost no non-specific killing was observed for MM1S cells. For KASUMI-1 cells, almost no killing was observed from the empty network of polynucleotides and scrambled aptamer, used as negative controls, (at most, approximately 20%). Specific cell killing was again seen for the aptamers alone after 3 hours incubation (unlike 24 hours in the previous experiment): approximately 50% killing of KASUMI-1 cells. During the 3-hour incubation, 3x3v1 nets did not appear to potentiate this effect. However, TRD1 potentiated the killing effect to approximately 90% cell killing.

FIG. 14B shows the results of kinetic fluorescent assays of daunorubicin release over time. Despite both “ABWT050902 Alone, With Daunorubicin” and “ABWT050902 on TRD1” conditions beginning the assay at the same time, the former began the assay with higher fluorescence of 400.0+ RFUs compared to the latter. This is because more free daunorubicin was in the supernatant buffer of “ABWT050902 Alone, with Daunorubicin” compared to “ABWT050902 on TRD1”. RFUs for the former took longer to reach a plateau than the latter, suggesting that more daunorubicin was released for a longer period compared to aptamers immobilized on DNA Net TRD1.

FIG. 15 shows the IC₅₀ of the killing of AML cells by aptamers loaded with daunorubicin, free and immobilized on networks of polynucleotides. Across aptamers, aptamer installation on the network of polynucleotides potentiates IC₅₀. The extent of the potentiation depends on a network of polynucleotides, as demonstrated by the comparison of IC₅₀s between aptamers alone (i.e., “Free” in the data table), 3x3v1 nets, and TRD1 tiles.

FIG. 16A illustrates a scheme of Protein G-docking oligo conjugate formation and docking to a network of polynucleotides. FIG. 16B demonstrates oligo-Protein G conjugate docking to 3x3v1 nets. 3x3v1 nets bear 27 docking sites: 3 sites per Holliday junction, with 5 nm spacing between each site at the Holliday junction. 1) 3x3v1 net alone; 2) 3x3v1 net+oligo alone (1:1 molar ratio); 3) 3x3v1 net+conjugate (1:1 molar ratio); 4) 3x3v1 net+conjugate (1:2 molar ratio); 5) 3x3v1 net+conjugate (1:4 molar ratio); 6) 3x3v1 net+conjugate (1:8 molar ratio); 7) 3x3v1 net+conjugate (1:16 molar ratio); 8) 3x3v1 net alone.

FIG. 16C shows optimization of oligo-Protein G conjugate docking ratio to 3x3v2 nets. 3x3v2 nets bear 9 docking sites: 1 site per Holliday junction, with 15 nm spacing between sites. 1) 3x3v2 net alone; 2) 1x molar ratio of the conjugate to 3x3v2 net; 3) 2x molar ratio of the conjugate to 3x3v2 net; 4) 4.5x molar ratio of the conjugate to 3x3v2 net; 5) 9x molar ratio of the conjugate to 3x3v2 net; 6) 18x molar ratio of the conjugate to 3x3v2 net; 7) 3x3v2 net alone.

FIG. 16D shows the Protein G activity after conjugation to docking oligo and immobilization onto 3x3v2 nets; surface plasmon resonance (SPR) binding assays against mouse IgG immobilized on Biacore CM5 sensor chips. A) 3x3v2 nets alone (green sensor trace) do not demonstrate binding to mouse IgG immobilized on Biacore CM5 sensor chips (the trace gives a bulk shift pattern characteristic of non-binding fluid boluses as they flow through the sensor channel). The oligo-Protein G conjugate alone (blue sensor trace) demonstrates binding (association) and dissociation to mouse IgG. Oligo-Protein G conjugates on 3x3v2 nets (red trace) also demonstrate binding. The amount of oligo Oligo-Protein G conjugate was equivalent between the “Conjugate” and “3x3v2-Conjugate” conditions, suggesting that upon immobilization to 3x3v2 nets, the conjugates both confer IgG Fc domain binding ability to the nets and experience avidity improvement to the IgG Fc domains. B) A column chart of the data docked in A. 3x3v2 nets alone demonstrate almost no RUs when flowed over mouse IgG, whereas Oligo-Protein G conjugates had a relative response of ˜145 RUs, and ˜170 RUs when immobilized on 3x3v2 nets.

FIG. 17A shows K_{D} determinations of 45-PTR-02-A15 against CD117 and SARS-CoV-2 N protein via SPR. A) Binding of 45-PTR-02-A15 to CD117 across concentrations; K_{D}=0.25 nM. B) Binding of 45-PTR-02-A15 to SARS-CoV-2 N protein across concentrations; K_{D}=4.7 nM. FIG. 17B shows a schematic of docking sites in DNA tile PTR-86. FIG. 17C shows a schematic of docking sites in DNA tile PTR-45.

FIG. 17D shows the ability of PTR86 to kill various AML cell lines as a function of biomarker expression and extent of targeting. Monotargeted PTR86 02:02 (only targeted to CD 117 via aptamer ABWT050902) demonstrated almost no killing of HL-60 cells (no CD117 expression) and significant (<30%) killing of HEL92.1.7 and KASUMI-1 cells (high CD 117 expression). Similarly, Monotargeted PTR86 30:30 (only targeted to CD123 via aptamer CY30) demonstrated slight killing of HEL92.1.7 cells (low CD123 expression) and significant (<30%) killing of HL-60 and KASUMI-1 cells (high CD123 expression). Dual-targeted PTR86 02D1:30D2 (binding CD117 via aptamer ABWT050902 and binding CD123 via aptamer CY30) showed significant cell killing of all three cell lines. Notably, dual-targeted PTR86 02D1:30D2 showed killing of KASUMI-1 cells that was superior to the monotargeted Networks of polynucleotides. Moreover, dual-targeted PTR86 02D1:30D2 demonstrated killing of KASUMI-1 cells that was superior to PTR86 02D1:30D2 killing of HL60 and HEL92.1.7 cells.

FIG. 17E shows the differences in abilities of monotargeted and dual-targeted networks of polynucleotides in killing various AML cell lines. Comparing high CD117-expressing cell lines HEL92.1.7 and KASUMI-1, monotargeted PTR86 02:02 (targeted only to CD117; 76 ABWT050902 aptamers) was able to kill 23.3% more KASUMI-1 cells than HEL92.1.7 cells: an effect caused in part by the differences of CD117 expression levels and cellular sensitivities to daunorubicin between the two cell lines. Comparing high CD123-expressing cell lines HL-60 and KASUMI-1, monotargeted PTR86 30:30 (targeted only to CD123; 76 CY30 aptamers) showed an increase of KASUMI-1 cell killing of 22.8% over HEL92.1.7: an effect caused in part by the differences between the cell lines in terms of CD123 expression levels and cellular sensitivities to daunorubicin. Dual-targeted PTR86 02D1:30D2 (targeted to both CD117 and CD123 via 38 ABWT050902 aptamers and 38 CY30 aptamers, respectively), demonstrated and increase in KASUMI-1 cell killing of 40.0% over HL-60 cells, and 35.0% over HEL92.1.7 cells; an additional 17.8% killing enhancement of KASUMI-1 cells versus HL-60 cells when comparing PTR86 30:30 and PTR86 02D1:30D2, and an additional 11.7% killing enhancement of KASUMI-1 cells versus HEL92.1.7 cells when comparing PTR86 02:02 and PTR86 02D1:30D2.

FIG. 18 shows the impact of ABWT050902 on KASUMI-1 cell viability when either used alone or when immobilized on two different networks of polynucleotides: PTR45 or PTR86. These results demonstrate that ABWT050902 aptamers alone and ABWT050902 aptamers on PTR45 had similar biological efficacies, killing KASUMI-1 cells with similar IC₅₀ values: 104.3 μM and 78.8 μM, respectively. The IC₅₀ for ABWT050902-PTR86 was calculated to be 78.8 μM, which was a 39.4-fold improvement as compared to IC₅₀ of ABWT050902-PTR45, which was 2.7 μM (compared to 1.32-fold improvement for ABWT050902-PTR45 over ABWT050902 aptamer alone).

FIG. 19A shows a scheme of docking Fc domain-bearing binders to networks of polynucleotides via a Protein G-docking oligo conjugate. FIG. 19B shows a scheme of immobilizing various docking oligo-protein conjugates to networks of polynucleotides via Watson-Crick base-pairing.

FIG. 20 shows a scheme of the inter-cluster distance (ICD_{E}) formula and elucidation.

FIG. 21 is a schematic representation of intra-cluster and inter-cluster spacing of antigens on the target cell(s), and networks of polynucleotides with intra-cluster and inter-cluster patterns that match that of the antigens on a target analyte.

FIG. 22 shows a workflow of designing the spatial pattern of the binders on the networks of polynucleotides, when the antigen distribution profile on target cells is unknown.

FIG. 23 illustrates the analysis of the widest dimension of CD117/c-KIT.

FIG. 24 illustrates the analysis of the widest dimension of CD123/IL3RA.

FIG. 25A-25C show schematics of various arrangements of different binder clusters on a biopolymer.

FIG. 25D shows an antigen comprising three different epitopes (represented by solid spheres), which are recognized by three different aptamers on a biopolymer.

FIG. 26 illustrates an alternative process for treating a subject using an artificial biopolymer complex according to various embodiments of the present disclosure.

FIG. 27 illustrates a method for conjugating a non-intercalating chemotherapy, e.g., monomethyl auristatin E (MMAE), onto an oligonucleotide capable of hybridizing to docking sites on polynucleotide networks.

The details of various embodiments of the invention are set forth in the description below. Other features, objects, and advantages of the invention will be apparent from the description and the drawings, and from the claims.

### Overview

Disclosed herein are artificial biopolymer complexes comprising networks of polynucleotides (i.e., polynucleotide nanostructures) that can deliver therapeutic effects to a subject in need thereof. The networks comprise loci (e.g., uniquely addressable loci) that are separated by predetermined inter-binder distances such that the binders are positioned on the network of polynucleotides in a predetermined two-dimensional or three-dimensional spatial pattern that matches a two-dimensional or three-dimensional spatial pattern of the antigens on the target analyte. In some embodiments, a biopolymer complex disclosed herein further comprises one or more therapeutic agents attached to the network of polynucleotides. In some embodiments, the biopolymer complex comprise different antigen binders (docked on the network of polynucleotides) that recognize different antigens on target analytes.

Also disclosed herein are multispecific polynucleotide nanostructures and techniques that use multispecific polynucleotide nanostructures as multimolecular recognition entities for the detection of antigens. The design of the multispecific polynucleotide nanostructures takes advantage of a polyvalent binding strategy to bind to multiple molecules, including (i) a target pathological biological entity (e.g., a tumor cell), and (ii) a target of the immune system (e.g., immune cells such as white blood cells), with high binding avidity. This enables targeted immunotherapy via triggering the immune system to kill or remove the target antigens and, optionally to introduce toxins/therapeutics to the target antigens.

A diversity of immunomodulatory agents, including tumor-associated antigens, adjuvants, cytokines, and immunomodulators, have been explored for their ability to induce a cascading adaptive immune response. Immunostimulatory and immunomodulatory nucleic acids are common adjuvants in the immunotherapy of various diseases. For example, CpG and poly I:C are capable of reacting with different TLR-like receptors to elicit a strong systemic immune reaction, and they can also be used as vaccine components for immunotherapy. Combining nanotechnology with immunotherapy aims to reinforce the cancer-immunity cycle, via potentiating key steps in the immune reaction cascade, namely antigen release, antigen processing, antigen presentation and immune cell-mediated tumor killing. Combinations of nano-immunotherapy can be realized via three targeting strategies, i.e., by targeting cancer cells, targeting the tumor immune microenvironment and targeting the peripheral immune system. However, nano-immunotherapy systems face issues concerning stability in physiological media, protein corona formation, accumulation in the target tissue, and sensitivity/specificity for delivering cytotoxicity only to target cancer cells.

To address these limitations and others, the multispecific polynucleotide nanostructures of the present disclosure make use of a network of polynucleotides that provide a structure for a defined spacing of binding ligands, aka., binders, (e.g., aptamers) to bind specifically to (i) antigen clusters on the outer surface of a target pathological biological entity, such as a tumor cell, and (ii) biomarker clusters on the outer surface of a target of the immune system, such as a white blood cell. The defined spacing includes (i) intermolecular spacing of target antigens on a cell surface or intermolecular spacing of biomarkers on immune cells, (ii) intramolecular spacing of target epitopes on an antigen such as a multimeric surface protein or other multimeric target molecule, or intramolecular spacing of epitopes on a biomarker on an immune cell and/or (iii) spacing between antigen clusters or spacing between biomarker clusters. Advantageously, the defined spacing allows for the network of polynucleotides to be constructed to bind specifically to multiple targets on the surface of a target pathological biological entity and multiple targets on the surface of a target of the immune system. This specific binding of the network of polynucleotides to antigen clusters increases the sensitivity and specificity of the nano-immunotherapy. Additionally, the network of polynucleotides can be the relatively large (e.g., greater than 25 nm in length, (e.g., 1-2000 nm, 2-1500 nm, 2-1000 nm, 2-900 nm, or 4-500 nm), which provides a highly customizable framework for carrying payloads such as therapeutic molecules, at varying capacities. Moreover, the relatively large (and more complex) network of polynucleotides contain multiple helical domains packed into bundles (arranged in, for example, square or rhombus lattices). This close-packed structure in the network of polynucleotides provides structural integrity when the multispecific polynucleotide nanostructures are exposed to a variety of nucleases.

The structural integrity of the polynucleotide nanostructures can be further enhanced through nucleotide cross-linking techniques. Methods for cross-linking nucleotides include the use of chemical agents including but not limited to nitrogen mustards, cisplatin, chloroethyl nitroso urea, carmustine, psoralens, mitomycin C, nitrous acid, and bifunctional aldehydes. Alternatives to chemical methods are biological agents including enzymes such as DNA ligase and photo-crosslinking by exposure to ultraviolet (UV) light.

The pharmacokinetics and pharmacodynamics of therapeutics play an important role in their efficacy. The polynucleotide nanostructures can be functionalized to modulate these properties. The characteristics such as size, density, molecular weight, surface chain density as well as conformations of the functional groups can be customized to ensure they do not interfere with the binding agents attached to the polynucleotide scaffold. Polymers such as poly(ethylene glycol) (PEG), poly(2-oxazoline) (POx), and poly(zwitterions) are commonly used in poly nucleotide nanostructures to further improve their resistance to nucleases by avoiding detection. Alternatives to polymers include functionalization with peptides or proteins which allow the polynucleotide nanostructures appear as an endogenous structure or hydrophobic compounds such as lipids to enhance penetration of the blood-brain barrier.

One illustrative embodiment of the present disclosure is directed to an artificial biopolymer complex that includes a network of polynucleotides comprising connector chains of polynucleotides attached to a structural chain of polynucleotides, a first set of binders attached to a first surface of the network of polynucleotides, and a second set of binders attached to a second surface of the network of polynucleotides. The first set of binders bind to antigens of a target analyte; and the first set of binders are attached at uniquely addressable loci on the structural chain via the connector chains, where the uniquely addressable loci are separated by predetermined inter-binder distances such that the first set of binders are positioned on the network of polynucleotides in a predetermined two-dimensional or three-dimensional spatial pattern that matches a two-dimensional or three-dimensional spatial pattern of the antigens on the target analyte. The second set of binders bind to biomarkers of a white blood cell; and the second set of binders are attached at uniquely addressable loci on the structural chain via the connector chains, where the uniquely addressable loci are separated by predetermined inter-binder distances such that the second set of binders are positioned on the network of polynucleotides in a predetermined two-dimensional or three-dimensional spatial pattern that matches a two-dimensional or three-dimensional spatial pattern of the biomarkers on the immune cells.

Another illustrative embodiment of the present disclosure is directed to an artificial biopolymer complex that includes a network of polynucleotides comprising structural units connected to one another via a series of arms and junctions, a first set of binders attached to a first surface of the network of polynucleotides, and a second set of binders attached to a second surface of the network of polynucleotides. Intersections of two or more (e.g., three or more) arms form the junctions. The first set of binders bind to antigens of a target analyte; and the first set of binders are attached at uniquely addressable loci on the arms forming the junctions, where the uniquely addressable loci are separated by predetermined inter-binder distances such that the first set of binders are positioned on the network of polynucleotides in a predetermined two-dimensional or three-dimensional spatial pattern that matches a two-dimensional or three-dimensional spatial pattern of the antigens on the target analyte. The second set of binders bind to biomarkers of a white blood cell; and the second set of binders are attached at uniquely addressable loci on the arms forming the junctions, where the uniquely addressable loci are separated by predetermined inter-binder distances such that the second set of binders are positioned on the network of polynucleotides in a predetermined two-dimensional or three-dimensional spatial pattern that matches a two-dimensional or three-dimensional spatial pattern of the biomarkers on the immune cells.

### Artificial Biopolymer Complexes

The artificial biopolymer complexes described herein provide a dual-faced display of binders—a first surface (e.g., a top surface) displays a number of binders (in some instances sets of clustered antigen binders, e.g., each target a tumor specific antigen such as CD33, CD123, CD117, which are highly correlated with acute myeloid leukemia disease), and a second surface (e.g., a bottom surface) displays a number of binders (in some instances sets of clustered biomarker binders, e.g., each target a biomarker on an immune cell, such as CD3, which is a T-cell receptor). The intent of the dual-faced display (a multispecific polynucleotide nanostructure) is to facilitate immunotherapy by activation of a subject's own immune system to a pathological biological entity, such as cancer. Cancer cells can evade immune surveillance in the body. However, immune checkpoint inhibitors can interrupt this evasion and activate or enhance the antitumor activity of immune cells, such as T-cells. Other mechanisms for promoting antitumor T-cell function are the targeting of costimulatory molecules expressed on the surface of T cells, such as 4-1BB, OX40, inducible T-cell costimulator and glucocorticoid-induced tumor necrosis factor receptor. In addition, CD40 targets the modulation of the activation of antigen-presenting cells, which ultimately leads to T-cell activation. Agonists of these various molecules such as costimulatory molecules have demonstrated promising results in preclinical and early-phase trials.

The antigen binding side of the artificial biopolymer complexes will have a much stronger binding to the target analyte, e.g., tumor cell, as compared to the immune cells, e.g., white blood cells, because immune cells will unlikely display all targeted biomarkers. This will allow for activation or enhancement of the immune cells but also allow for undocking of the complex from the immune cell to facilitate the cascade of the immune response and additional therapies such as introduction of a therapeutic agent to the target analyte. The artificial biopolymer complexes are designed with multi-valency. (density/distance of binders) so that the antigen binding side of the complex binds the target analyte but does not bind immune cells, even if the immune cells express the antigens (i.e., the patterns between binders and antigens will not match). This provides a robust sensitivity and specificity for the target analyte. Additionally, the polynucleotide nanostructures of the complexes may be designed with a curvature such that the biomarker binding side of the complexes are at least partially enveloped by the second surface. Binding of the target analyte can flatten the complexes so that the biomarker binding side is exposed to recruit an immune cell and trigger immunotherapy. This ensures that complexes primarily bind with the target analyte and do not inadvertently bind with immune cells without binding to target analytes.

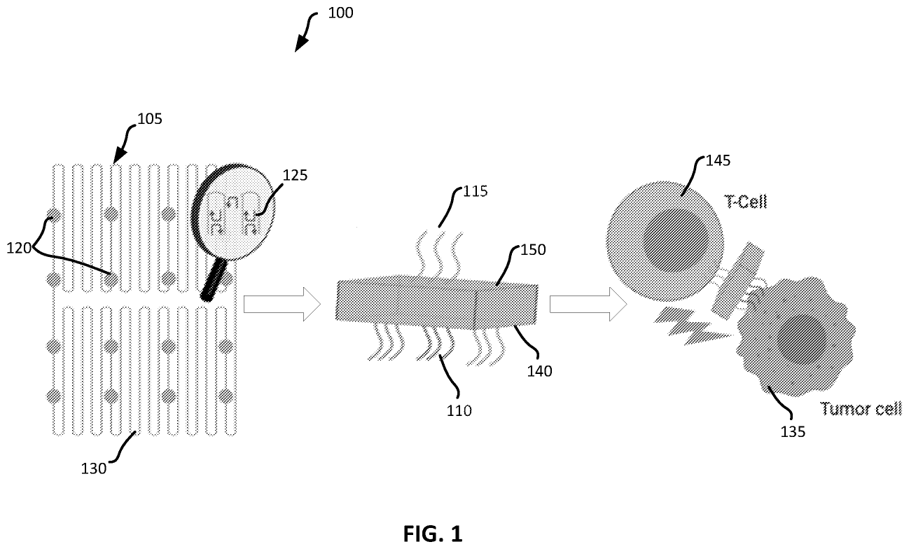

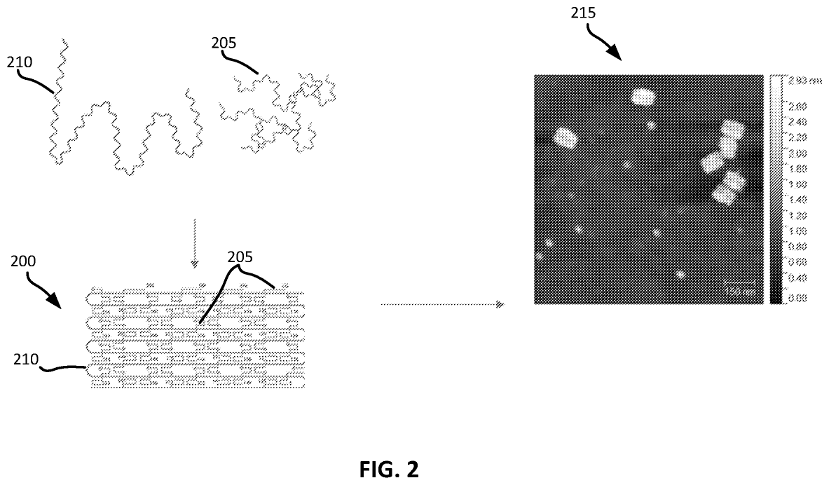

FIG. 1 illustrates an artificial biopolymer complex 100 that include multispecific polynucleotide nanostructures as multimolecular recognition entities for the detection of antigens according to various embodiments of the present disclosure. The artificial biopolymer complex 100 comprises a network of polynucleotides 105, a first set of binders 110, and a second set of binders 115. As shown in FIG. 2, a network of polynucleotides 200 (e.g., network of polynucleotides 105) may comprise connector chains 205 of polynucleotides 205 attached to a structural chain 210 of polynucleotides. The connector chains 205 are shorter in length than the structural chain 210. For example, the structural chain 210 may be greater than 25 nm in length; whereas each connector chain may be less than 10 nm. The atomic force microscopy image (AFM) 215 shows the network of polynucleotides 200 with binders attached. The connector chains 205 and/or structural chain 210 may be single-stranded DNA or RNA (ssDNA or ssRNA). At least a portion of each chain of the connector chains 205 is complementary to at least a portion of the structural chain 210, and the complementary portions of the connector chains 205 and structural chain 210 are hybridized (combining two complementary ssDNA or ssRNA molecules) to connect the connector chains 205 to the structural chain 210.

With respect back to FIG. 1, the network of polynucleotides 105 provides uniquely addressable anchor loci 120 for displaying the same or different binders at each anchor location 120. The uniquely addressable anchor loci 120 may be located on the connector chains 125 and/or the structural chain 130. The network of polynucleotides 105 is functionalized by attaching the first set of binders 110 and the second set of binders 115 at the uniquely addressable loci 120 on various surfaces of the network of polynucleotides 105. In some instances, the first set of binders 115 bind to antigens of a target analyte 135 (e.g., tumor cells) and are attached to a first surface 140 of the network of polynucleotides 105 at uniquely addressable loci 120 on the structural chain 130 via the connector chains 125. In some instances, the second set of binders 115 bind to biomarkers of immune cells 145 (e.g., T-cells) and are attached to a second surface 150 of the network of polynucleotides 105 at uniquely addressable loci 120 on the structural chain 130 via the connector chains 125.

As used herein, “immune cells” include T cells, NK cells, macrophages, alveolar macrophages, Kupffer cells, microglial cells, T_{reg} cells, T_{Helper} cells, antigen presenting cells (APCs) and the like, that have cytotoxicity or assist in generation of cytotoxic effects.

By attaching the first set of binders 110 and the second set of binders 115 at the uniquely addressable loci 120 on various surfaces of the network of polynucleotides 105, the artificial biopolymer complex 100 is capable of recruiting and engaging immune cells (cytotoxic T cells, natural killer (NK) cells, or the like) 140 for killing the tumor cells 130.

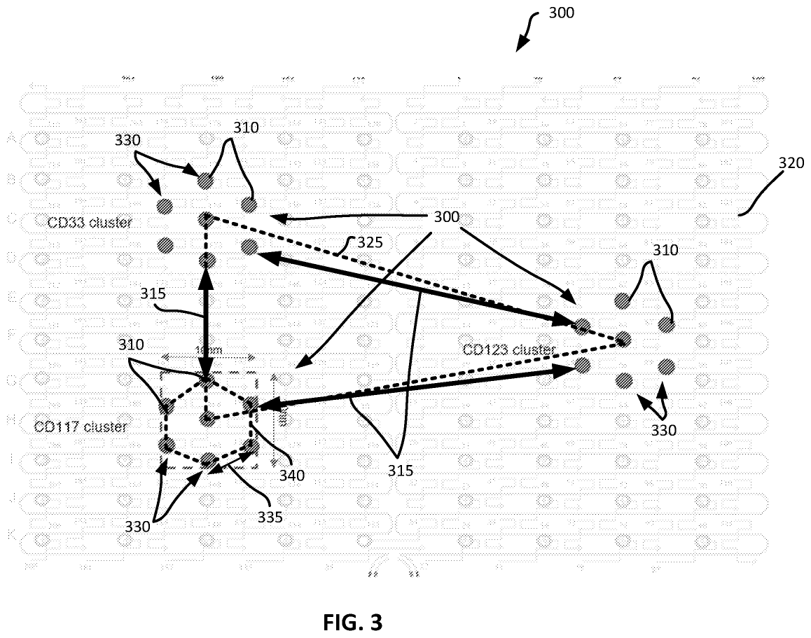

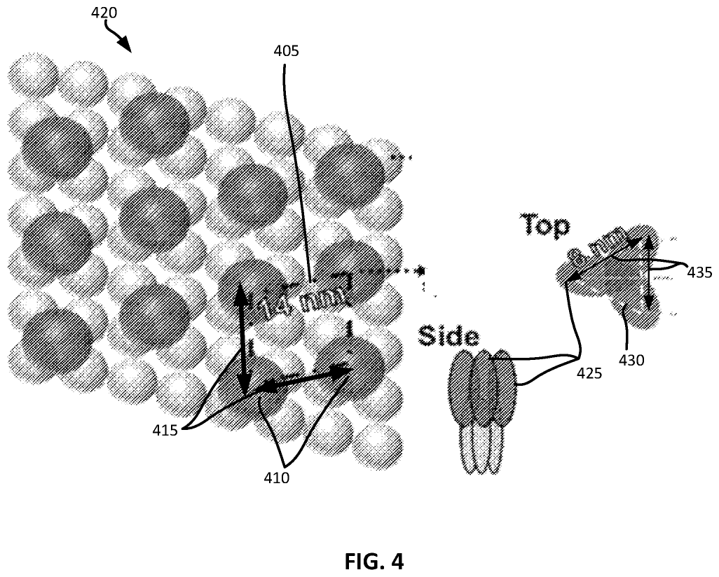

As shown in FIG. 3, the uniquely addressable loci 300 (e.g., loci 120) of the first set of binders 310 (e.g., binders 110) are separated by predetermined inter-binder distances 315 such that the first set of binders 310 are positioned on the network of polynucleotides 320 (e.g., network of polynucleotides 105) in a predetermined two-dimensional or three-dimensional spatial pattern 325 that matches a two-dimensional or three-dimensional spatial pattern of the antigens on the target analyte. As shown in FIG. 4, the two-dimensional or three-dimensional spatial pattern 405 of the antigens 410 is defined by intermolecular spacing 415 of the antigens 410 on a surface of the target analyte 420. The predetermined inter-binder distances 315 of the uniquely addressable loci of the first set of binders 310 match the intermolecular spacing 415 of the antigens 410 such that the first set of binders 310 on the first surface of the network of polynucleotides 320 align spatially with the antigens 410 on the surface of the target analyte 420.

More specifically, each of the antigens 410 is (i) a length and width in angstroms or nanometers from other antigens 410 on the target analyte 420 or (ii) a length, width, and depth from the other antigens 410 on the target analyte 420, which define the intermolecular spacing 415 of the antigens 410. Each of the binders of the first set of binders 310 is (i) a length and width in angstroms or nanometers from other binders of the first set of binders 310 on the network of polynucleotides 320 or (ii) a length, width, and depth in angstroms or nanometers from the other binders of the first set of binders 310 on the network of polynucleotides 320, which defines the predetermined inter-binder distances 315 of the uniquely addressable loci 300 of the first set of binders 310. The predetermined inter-binder distances 315 of the uniquely addressable loci 300 of the first set of binders 310 match the intermolecular spacing 415 of the antigens 410 such that the first set of binders 310 on the first surface of the network of polynucleotides 320 align spatially with the antigens 410 on the surface of the target analyte 420.

In some instances, each of the antigens 410 comprises one or more epitopes 425. The first set of binders 310 may be arranged in sets of clustered antigen binders. The binders 310 of each of the sets of clustered antigen binders are attached at uniquely addressable loci 330 (e.g., loci 110) on the structural chain via the connector chains. The uniquely addressable loci 330 are separated by predetermined intra-binder distances 335 such that each set of clustered antigen binders is positioned on the network of polynucleotides 320 in a predetermined two-dimensional or three-dimensional spatial pattern 340 that matches a two-dimensional or three-dimensional spatial pattern 430 of the one or more epitopes 425 on an antigen 410. The two-dimensional or three-dimensional spatial pattern 430 of the one or more epitopes 425 is defined by intramolecular spacing 435 of the one or more epitopes 425 on a surface of the antigen 410. The predetermined intra-binder distances 335 of the uniquely addressable loci 330 of the binders 310 of each of the sets of clustered antigen binders match the intramolecular spacing 435 of the one or more epitopes 425 such that the sets of clustered antigen binders on the first surface of the network of polynucleotides 320 align spatially with the epitopes 425 on the surface of the antigens 410.

More specifically, each of the one or more epitopes 425 is (i) a length and width in angstroms or nanometers from other epitopes of the one or more epitopes 425 on the antigen 410 or (ii) a length, width, and depth from the other epitopes of the one or more epitopes 425 on the antigen 410, which define the intramolecular spacing 435 of the one or more epitopes 425. Each of the binders 310 of each of the sets of clustered antigen binders is (i) a length and width in angstroms or nanometers from other binders 310 of each of the sets of clustered antigen binders on the network of polynucleotides 320 or (ii) a length, width, and depth in angstroms or nanometers from the other binders 310 of each of the sets of clustered antigen binders on the network of polynucleotides 320, which defines the predetermined intra-binder distances 335 of the uniquely addressable loci 330 of the binders 310 of each of the sets of clustered antigen binders. The predetermined intra-binder distances 335 of the uniquely addressable loci 330 of the binders 310 of each of the sets of clustered antigen binders match the intermolecular spacing 435 of the epitopes 425 such that each of the sets of clustered antigen binders on the first surface of the network of polynucleotides 320 align spatially with the epitopes 425 on the surface of the antigens 410. In certain instances, the predetermined intra-binder distances 335 of the uniquely addressable loci 330 of the binders 310 of each of the sets of clustered antigen binders is between 1 nm and 15 nm.

In a similar manner (not shown), the uniquely addressable loci (e.g., loci 120) of the second set of binders (e.g., binders 115) are separated by predetermined inter-binder distances such that the second set of binders are positioned on the network of polynucleotides (e.g., network of polynucleotides 105) in a predetermined two-dimensional or three-dimensional spatial pattern that matches a two-dimensional or three-dimensional spatial pattern of the biomarkers on the immune cells. The two-dimensional or three-dimensional spatial pattern of the biomarkers is defined by intermolecular spacing of the biomarkers on a surface of the immune cells. The predetermined inter-binder distances of the uniquely addressable loci of the second set of binders match the intermolecular spacing of the biomarkers such that the second set of binders on the second surface of the network of polynucleotides align spatially with the biomarkers on the surface of the immune cells.

More specifically, each of the biomarkers is (i) a length and width in angstroms or nanometers from other antigens biomarkers on the immune cells or (ii) a length, width, and depth from the other biomarkers on the immune cells, which define the intermolecular spacing of the biomarkers. Each of the binders of the second set of binders is (i) a length and width in angstroms or nanometers from other binders of the second set of binders on the network of polynucleotides or (ii) a length, width, and depth in angstroms or nanometers from the other binders of the second set of binders on the network of polynucleotides, which defines the predetermined inter-binder distances of the uniquely addressable loci of the second set of binders. The predetermined inter-binder distances of the uniquely addressable loci of the second set of binders match the intermolecular spacing of the biomarkers such that the second set of binders on the second surface of the network of polynucleotides align spatially with the biomarkers on the surface of the immune cells.

Moreover, each of the biomarkers comprises one or more epitopes or domains. The second set of binders may be arranged in sets of clustered biomarker binders. The binders of each of the sets of clustered biomarker binders are attached at uniquely addressable loci (e.g., loci 110) on the structural chain via the connector chains. The uniquely addressable loci are separated by predetermined intra-binder distances such that each set of clustered biomarker binders are positioned on the network of polynucleotides in a predetermined two-dimensional or three-dimensional spatial pattern that matches a two-dimensional or three-dimensional spatial pattern of the one or more epitopes on a biomarker. The two-dimensional or three-dimensional spatial pattern of the one or more epitopes is defined by intramolecular spacing of the one or more epitopes on a surface of the biomarker. The predetermined intra-binder distances of the uniquely addressable loci of the binders of each of the sets of clustered biomarker binders match the intramolecular spacing of the one or more epitopes such that the sets of clustered biomarker binders on the second surface of the network of polynucleotides align spatially with the epitopes on the surface of the biomarker.

More specifically, each of the one or more epitopes is (i) a length and width in angstroms or nanometers from other epitopes of the one or more epitopes on the biomarker or (ii) a length, width, and depth from the other epitopes of the one or more epitopes on the biomarker, which define the intramolecular spacing of the one or more epitopes or domains. Each of the binders of each of the sets of clustered biomarker binders is (i) a length and width in angstroms or nanometers from other binders of each of the sets of clustered biomarker binders on the network of polynucleotides or (ii) a length, width, and depth in angstroms or nanometers from the other binders of each of the sets of clustered biomarker binders on the network of polynucleotides, which defines the predetermined intra-binder distances of the uniquely addressable loci of the binders of each of the sets of clustered biomarker binders. The predetermined intra-binder distances of the uniquely addressable loci of the binders of each of the sets of clustered biomarker binders match the intermolecular spacing of the epitopes such that each of the sets of clustered biomarker binders on the second surface of the network of polynucleotides align spatially with the epitopes on the surface of the biomarker. In certain instances, the predetermined intra-binder distances of the uniquely addressable loci of the binders of each of the sets of clustered biomarker binders is between 1 nm and 15 nm.

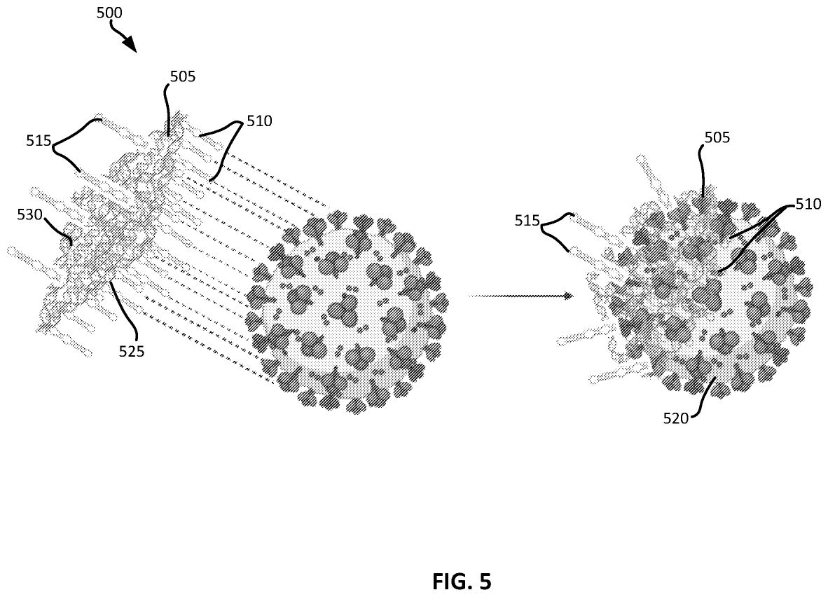

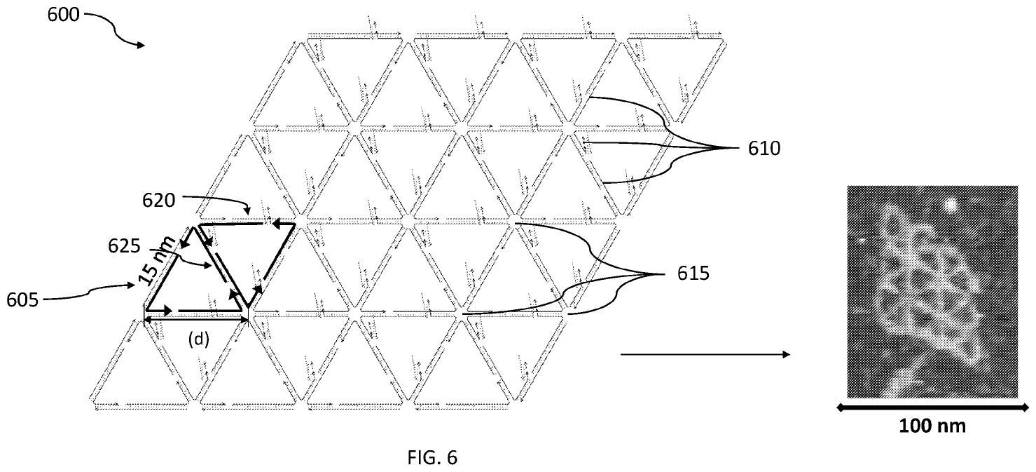

FIG. 5 illustrates an artificial biopolymer complex 500 that include multispecific polynucleotide nanostructures as multimolecular recognition entities for the detection of antigens according to various embodiments of the present disclosure. The artificial biopolymer complex 500 comprises a network of polynucleotides 505, a first set of binders 510, and a second set of binders 515. As shown in FIG. 6, a network of polynucleotides 600 (e.g., network of polynucleotides 505) may comprise structural units 605 connected to one another via a series of arms 610 and junctions 615. The lower right panel of FIG. 6 is an atomic force microscopy (AFM) image of a fully-assembled 3x3 net composed of 9 structural units as defined in paragraph [00140]. Intersections of three or more arms 610 form the junctions 615. Each of the structural units 605 have a predetermined shape (e.g., a triangle or rhombus) defined by one or more strands of polynucleotides. The one or more strands of polynucleotides may be ssDNA or ssRNA. At least a portion 620 of the one or more strands of polynucleotides of each structural unit is complementary to at least a portion 625 of the one or more strands of polynucleotides of another structural unit, and the complementary portions of the strands of the polynucleotides are hybridized to connect the structural units. The complementary portions of the strands of the polynucleotides form the arms 610 with a predetermined length (l), and the intersections of the three or more arms 610 form junctions 615 at a predetermined distance (d) from one another based on the predetermined length (l) of the arms 610.

The network of polynucleotides 600 provides uniquely addressable anchor loci 630 for displaying the same or different binders at each anchor location 630. The uniquely addressable anchor loci 630 may be located on the arms 610. With respect back to FIG. 5, the network of polynucleotides 505 is functionalized by attaching the first set of binders 510 and the second set of binders 515 at the uniquely addressable loci on various surfaces of the network of polynucleotides 505. In some instances, the first set of binders 510 bind to antigens of a target analyte 520 (e.g., tumor cells) and are attached to a first surface 525 of the network of polynucleotides 505 at uniquely addressable loci on the arms forming the junctions. In some instances, the second set of binders 515 bind to biomarkers of the immune cells (e.g., T-cells) and are attached to a second surface 530 of the network of polynucleotides 505 at uniquely addressable loci on the arms forming the junctions. By attaching the first set of binders 510 and the second set of binders 515 at the uniquely addressable loci on various surfaces of the network of polynucleotides 505, the artificial biopolymer complex 500 is capable of recruiting and engaging immune cells such as white blood cells for killing the tumor cells 520.

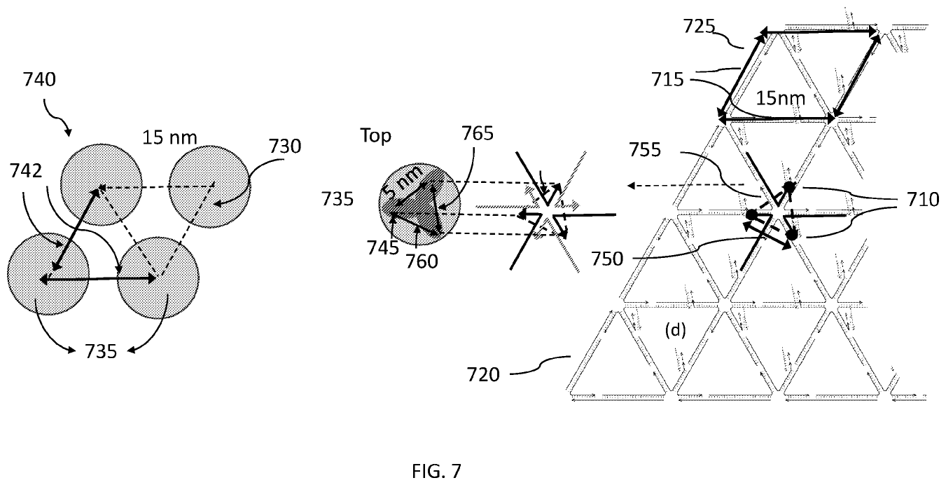

As shown in FIG. 7, the uniquely addressable loci (e.g., loci 630) of the first set of binders 710 (e.g., 510) are separated by predetermined inter-binder distances 715 such that the first set of binders 710 are positioned on the network of polynucleotides 720 (e.g., 505) in a predetermined two-dimensional or three-dimensional spatial pattern 725 that matches a two-dimensional or three-dimensional spatial pattern 730 of the antigens 735 on the target analyte 740. The two-dimensional or three-dimensional spatial pattern 730 of the antigens 735 is defined by intermolecular spacing 742 of the antigens 735 on a surface of the target analyte 740. The predetermined inter-binder distances 715 of the uniquely addressable loci of the first set of binders 710 match the intermolecular spacing 742 of the antigens 735 such that the first set of binders 710 on the first surface of the network of polynucleotides 720 align spatially with the antigens 735 on the surface of the target analyte 740.

More specifically, each of the antigens 735 is (i) a length and width in angstroms or nanometers from other antigens 735 on the target analyte 740 or (ii) a length, width, and depth from the other antigens 735 on the target analyte 740, which define the intermolecular spacing 742 of the antigens 735. Each of the binders of the first set of binders 710 is (i) a length and width in angstroms or nanometers from other binders of the first set of binders 710 on the network of polynucleotides 720 or (ii) a length, width, and depth in angstroms or nanometers from the other binders of the first set of binders 710 on the network of polynucleotides 720, which defines the predetermined inter-binder distances 715 of the uniquely addressable loci of the first set of binders 710. The predetermined inter-binder distances 715 of the uniquely addressable loci of the first set of binders 710 match the intermolecular spacing 742 of the antigens 735 such that the first set of binders 710 on the first surface of the network of polynucleotides 720 align spatially with the antigens 735 on the surface of the target analyte 740.

In some instances, each of the antigens 735 comprises one or more epitopes 745. The first set of binders 710 may be arranged in sets of clustered antigen binders. The binders 710 of each of the sets of clustered antigen binders are attached at uniquely addressable loci (e.g., loci 630) on the arms forming junctions. The uniquely addressable loci are separated by predetermined intra-binder distances 750 such that each set of clustered antigen binders are positioned on the network of polynucleotides 720 in a predetermined two-dimensional or three-dimensional spatial pattern 755 that matches a two-dimensional or three-dimensional spatial pattern 760 of the one or more epitopes 745 on an antigen 735. The two-dimensional or three-dimensional spatial pattern 760 of the one or more epitopes 745 is defined by intramolecular spacing 765 of the one or more epitopes 745 on a surface of the antigen 735. The predetermined intra-binder distances 750 of the uniquely addressable loci of the binders 710 of each of the sets of clustered antigen binders match the intramolecular spacing 765 of the one or more epitopes 745 such that the sets of clustered antigen binders on the first surface of the network of polynucleotides 720 align spatially with the epitopes 745 on the surface of the antigens 735.

More specifically, each of the one or more epitopes 745 is (i) a length and width in angstroms or nanometers from other epitopes of the one or more epitopes 745 on the antigen 735 or (ii) a length, width, and depth from the other epitopes of the one or more epitopes 745 on the antigen 735, which define the intramolecular spacing 765 of the one or more epitopes 745. Each of the binders 710 of each of the sets of clustered antigen binders is (i) a length and width in angstroms or nanometers from other binders 710 of each of the sets of clustered antigen binders on the network of polynucleotides 720 or (ii) a length, width, and depth in angstroms or nanometers from the other binders 710 of each of the sets of clustered antigen binders on the network of polynucleotides 720, which defines the predetermined intra-binder distances 750 of the uniquely addressable loci of the binders 710 of each of the sets of clustered antigen binders. The predetermined intra-binder distances 750 of the uniquely addressable loci of the binders 710 of each of the sets of clustered antigen binders match the intermolecular spacing 765 of the epitopes 745 such that each of the sets of clustered antigen binders on the first surface of the network of polynucleotides 720 align spatially with the epitopes 745 on the surface of the antigens 735. In certain instances, the predetermined intra-binder distances 50 of the uniquely addressable loci of the binders 710 of each of the sets of clustered antigen binders is between 1 nm and 15 nm.

In a similar manner (not shown), the uniquely addressable loci (e.g., loci 630) of the second set of binders (e.g., binders 515) are separated by predetermined inter-binder distances such that the second set of binders are positioned on the network of polynucleotides (e.g., network of polynucleotides 505) in a predetermined two-dimensional or three-dimensional spatial pattern that matches a two-dimensional or three-dimensional spatial pattern of the biomarkers on the immune cells. The two-dimensional or three-dimensional spatial pattern of the biomarkers is defined by intermolecular spacing of the biomarkers on a surface of the immune cells. The predetermined inter-binder distances of the uniquely addressable loci of the second set of binders match the intermolecular spacing of the biomarkers such that the second set of binders on the second surface of the network of polynucleotides align spatially with the biomarkers on the surface of the immune cells.

More specifically, each of the biomarkers is (i) a length and width in angstroms or nanometers from other antigens biomarkers on the immune cells or (ii) a length, width, and depth from the other biomarkers on the immune cells, which define the intermolecular spacing of the biomarkers. Each of the binders of the second set of binders is (i) a length and width in angstroms or nanometers from other binders of the second set of binders on the network of polynucleotides or (ii) a length, width, and depth in angstroms or nanometers from the other binders of the second set of binders on the network of polynucleotides, which defines the predetermined inter-binder distances of the uniquely addressable loci of the second set of binders. The predetermined inter-binder distances of the uniquely addressable loci of the second set of binders match the intermolecular spacing of the biomarkers such that the second set of binders on the second surface of the network of polynucleotides align spatially with the biomarkers on the surface of the immune cells.

Moreover, each of the biomarkers comprises one or more epitopes or domains. The second set of binders may be arranged in sets of clustered biomarker binders. The binders of each of the sets of clustered biomarker binders are attached at uniquely addressable loci (e.g., loci 630) on the arms forming junctions. The uniquely addressable loci are separated by predetermined intra-binder distances such that each set of clustered biomarker binders are positioned on the network of polynucleotides in a predetermined two-dimensional or three-dimensional spatial pattern that matches a two-dimensional or three-dimensional spatial pattern of the one or more epitopes on a biomarker. The two-dimensional or three-dimensional spatial pattern of the one or more epitopes is defined by intramolecular spacing of the one or more epitopes on a surface of the biomarker. The predetermined intra-binder distances of the uniquely addressable loci of the binders of each of the sets of clustered biomarker binders match the intramolecular spacing of the one or more epitopes such that the sets of clustered biomarker binders on the second surface of the network of polynucleotides align spatially with the epitopes on the surface of the biomarker.

More specifically, each of the one or more epitopes is (i) a length and width in angstroms or nanometers from other epitopes of the one or more epitopes on the biomarker or (ii) a length, width, and depth from the other epitopes of the one or more epitopes on the biomarker, which define the intramolecular spacing of the one or more epitopes or domains. Each of the binders of each of the sets of clustered biomarker binders is (i) a length and width in angstroms or nanometers from other binders of each of the sets of clustered biomarker binders on the network of polynucleotides or (ii) a length, width, and depth in angstroms or nanometers from the other binders of each of the sets of clustered biomarker binders on the network of polynucleotides, which defines the predetermined intra-binder distances of the uniquely addressable loci of the binders of each of the sets of clustered biomarker binders. The predetermined intra-binder distances of the uniquely addressable loci of the binders of each of the sets of clustered biomarker binders match the intermolecular spacing of the epitopes such that each of the sets of clustered biomarker binders on the second surface of the network of polynucleotides align spatially with the epitopes on the surface of the biomarker. In certain instances, the predetermined intra-binder distances of the uniquely addressable loci of the binders of each of the sets of clustered biomarker binders is between 1 nm and 15 nm.

### Network of Polynucleotides

The polynucleotides that make up the network can be either DNA, RNA, PNA, LNA, TNA, or DNA or RNA analogs (e.g., containing base analogs, sugar analogs, and/or a non-native backbone and the like. A biopolymer complex disclosed above comprises at least a network of polynucleotides and the binders displayed thereon.

The network of polynucleotides can take on a number of forms. In some embodiments, the network of polynucleotides form a plurality of Holliday junctions, with several double-stranded arms joined together. Networks of polynucleotides of this type are referred to as DNA nets in this disclosure. DNA nets can be designed as multiples of monomeric polynucleotide network structural units, as annotated in FIG. 6 by the dual triangle monomeric structure outlined by the bold arrows. Monomeric structural units are parallelograms composed of two interior equilateral triangles with 15 nm sides 630. Monomeric structural units are composed of a minimum of 6 polynucleotide sequences (core sequences). When the monomeric structural unit forms an edge or edges of the network of polynucleotides, the monomer contains polynucleotide sequences in addition to the 6 core polynucleotide sequences. These additional polynucleotide sequences hybridize to the core sequences on the edge of the network of polynucleotides, which makes the edges of the network of polynucleotides double-stranded DNA

Depending on the number of structural units, a DNA net can be a 2x2 net (4 structural units), a 3x3 net (9 structural unit), a 4x4 net (16 structural units), and so on. Depending on the number of the docking sites at the Holliday junctions, a DNA net may be named with different versions. For example, 3x3v1 means that there are 3 docking sites at each Holliday junctions, and 3x3v2 means that there is one docking site at each Holliday junctions, and so on. One illustration of the 4x4 net is shown in FIG. 6.

In some embodiments, the network of polynucleotides takes in origami format (“DNA origami”), which may take in various shapes such as a tile, a tube, or a tetrahedron, and the like. A DNA origami comprises connector strands and structural strands. One illustration of the networks of polynucleotides based upon DNA origami is shown in FIG. 2 and FIG. 4. TRD1, TRD2, PTR86 and PTR45 are all DNA origamis.

### Binders

An artificial biopolymer complex include binders attached to uniquely addressable loci on the network of polynucleotides described above. A binder attached to the network of polynucleotides can be an aptamer (e.g., single-stranded DNA (SSDNA), double-stranded (dsDNA), ssXNA, or dsXNA), antibodies, a peptide, a nanobody, an antibody mimic (e.g., an Affimer® or a molecularly-imprinted polymer), or a small analyte binder. Aptamers are short sequences of artificial DNA or RNA that bind a specific target molecule. In instances in which the binders are aptamers, the aptamers may be developed and selected via systematic evolution of ligands by exponential enrichment (SELEX), also referred to as in vitro selection or in vitro evolution. SELEX is a combinatorial chemistry technique in molecular biology for producing oligonucleotide libraries of either ssDNA, ssRNA, or ssRNA/ssDNA bearing chemical modifications, such as 2′-moieties or non-natural bases, that can contain specific oligonucleotides capable of specifically binding to a target ligand or ligands (i.e., aptamers).

### Antigen Binders

The antigen binders (also referred to as the first set of binders) bind to antigen molecules on target analytes (e.g., tumor cells). As used herein, the term “antigen” refers to any molecule (e.g., a protein molecule) that is expressed on the surface of a target analyte and are recognized by a binder disclosed herein. In some embodiments the antigen binders are aptamers. In some embodiments, the aptamers bind to one or more antigens that are specific for a particular tumor type, for example, acute myelogenous leukemia (AML). Non-limiting examples of tumor specific antigens include CD3, CD117/c-KIT, CD123, CD19, CD20, CD22, CD30, CD33, CD138, CD244, CEA, EGFR/HER1/erbB1, HERer2/erbB2, and the like.

Exemplary CD117-binding aptamers are as follows. All sequences are listed in the orientation from 5′ to 3′.

ABWT050902:
(SEQ ID NO: 1)
ATCCAGAGTGACGCAGCACCGCCCATGTCGAATAGGTGCCACGTAGGG
ACTTGGACACGGTGGCTTAGT
ABWT050904:
(SEQ ID NO: 2)
ATCCAGAGTGACGCAGCACGAGCCTTGTGTCGTTCGGAGTGCTGGTCC
ACGACTACTATTGGACACGGTGGCTTAGT
#1
(SEQ ID NO: 3)
GAGGCATACCAGCTTATTCAAGGGGCCGGGGCAAGGGGGGGGTACCGT
GGTAGGACATAGTAAGTGCAATCTGCGAA
Sequences of exemplary CD123-binding aptamers
are listed below from 5′ to 3′:
ZW25:
(SEQ ID NO: 4)
TGCGTGTGTAGTGTGTCTGGGCTACATCGATGAGCTGCCTAGGGTCCC
TCTTAGGGATTTGGGCGGG
CY30:
(SEQ ID NO: 5)
TGCGTGTGTACTGTGTCTGGTCCCGTAGCTACTAGCGAACTCCCTGCC
TCTTAGGGATTTGGGCGG

One skilled in the art would recognize that any aptamer that can bind to the target antigen (e.g., CD117 or CD123) can be used in a network of polynucleotides disclosed herein. In some embodiments aptamer variants having nucleotide sequences that have one, two, three, four, five, six, seven, eight, nine, ten or more mutations as compared to the sequence of one of the above aptamers can also be used as binders in the biopolymer complex. In some embodiments, these aptamer variants have substantially similar binding affinity as one of the aptamers disclosed above. As used herein, substantially similar refers to that the binding affinity of an aptamer variant to a target antigen is at least 50%, at least 60%, at least 70%, at least 80%, or at least 90% of the binding affinity of a reference aptamer (e.g., one of the five aptamers disclosed above). The binding affinities can be assessed using methods well known in the art, and also those described in the Examples of this disclosure.

In some embodiments the binders are antibodies. In some embodiments, the antibodies bind one or more antigens that are specific for a particular tumor type, for example, acute myelogenous leukemia (AML). Non-limiting examples of antibodies that can be used include antibodies against CD3, CD117, CD123, CD19, CD20, CD22, CD30, CD33, CD138, CD244, CEA, EGFR, Her2, and the like.

### Biomarker Binders

Biomarker binders bind to biomarkers on immune cells. As used herein, the term “biomarker” refers to any molecules (e.g., a protein) that is expressed on immune cells and can be recognized by a binder displayed in the network of polynucleotides. Biomarker binders are also referred to as the second set of binders in this disclosure. In some embodiments, immune cells refer to non-tumor cells that have cytotoxicity, i.e., cells that, when contacting the target cells or brought in proximity to the target cells, can inhibit cell proliferation and/or induce cell death. In some embodiments, the immune cells are immune cells that have cytotoxicity, for example, T cells, NK cells, macrophages, alveolar macrophages, Kupffer cells, microglial cells, T_{reg} cells, T_{Helper} cells, antigen presenting cells (APCs), and the like. In some embodiments, the immune cells can be mesenchymal stem cells, neural stem cells, cells that have therapeutically-relevant characteristics, such as predilections for migration to and through tissues undergoing inflammatory processes, tissues that are cancerous, along forming, formed, and angiogenic blood vessels, etc.

In some embodiments, the artificial biopolymer complex comprises antigen binders. In some embodiments, the artificial biopolymer complex comprises biomarker binders. In some embodiments, the artificial biopolymer complex comprises both antigen binders and biomarker binders, and the antigen binders and biomarker binders are attached to opposite surfaces of network of polynucleotides of the biopolymer complex. One illustrative example of a biopolymer complex comprising both antigen binders and biomarker binders is shown in FIG. 5.

### Displaying Binders on the Network of Polynucleotides

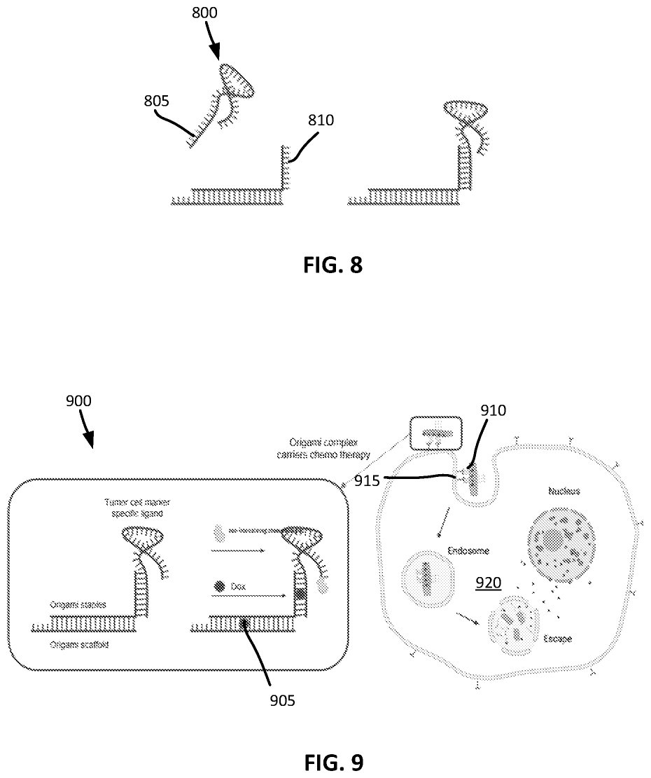

Binders can be attached (docked) to the network in a number of ways. In some embodiments, a binder hybridizes to an anchor sequence in the network. As shown in FIG. 8, the binders 800 may be extended to have a complementary sequence 805 to the anchor sequence 810 (e.g., a sequence in the arms or the connector chains of the network of polynucleotides).

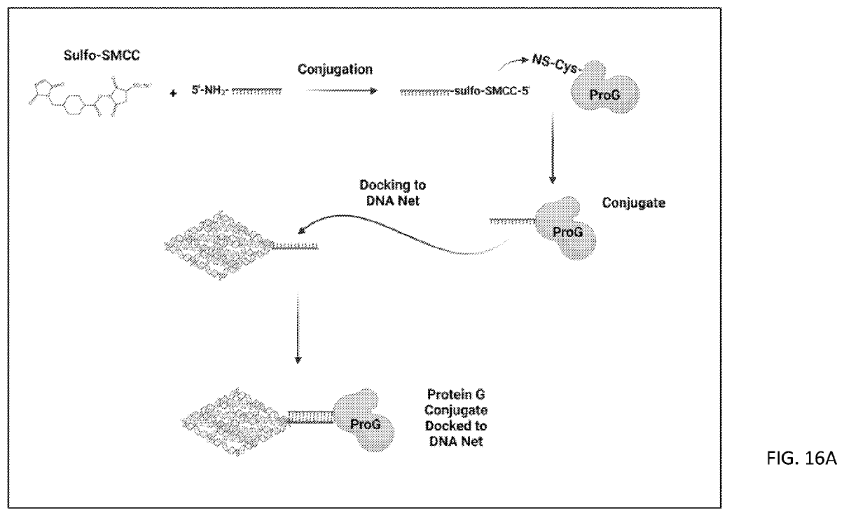

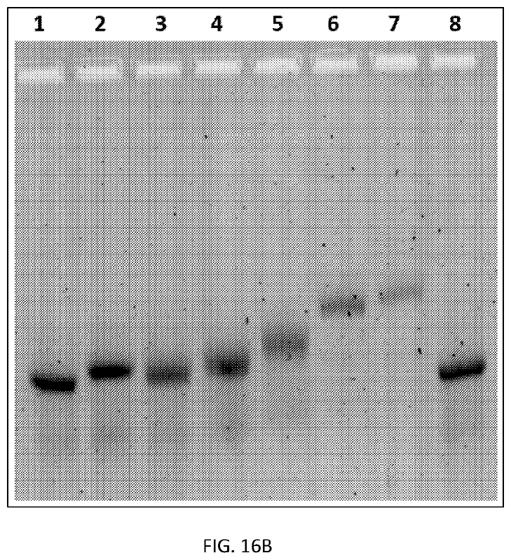

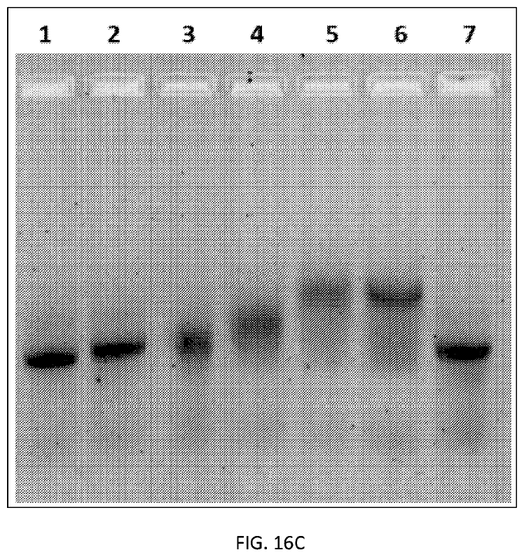

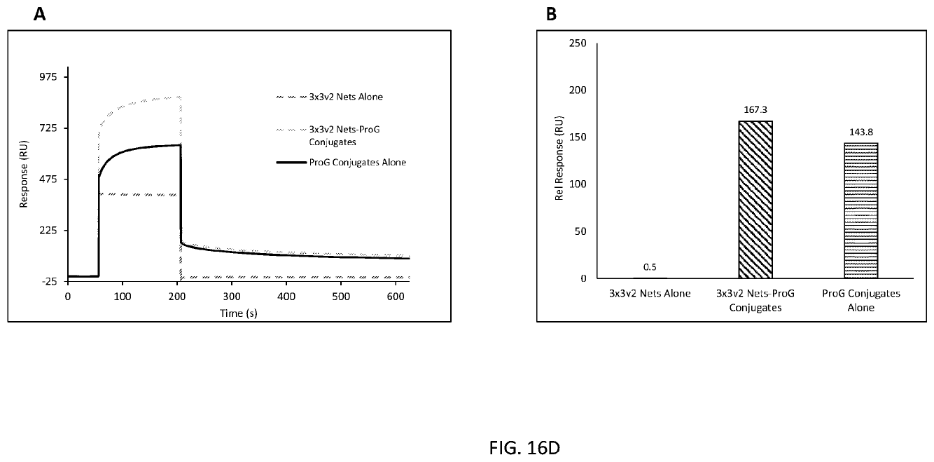

In some embodiments, the binders are proteins, for example, antibodies, or other Fc-containing peptides, Fc-containing protein domains, or Fc-containing non-antibody proteins. In some embodiments, the proteins are conjugated to oligonucleotides that are complementary to the anchor sequence, and the oligonucleotide-protein conjugates are then docked to the network of polynucleotides through the hybridization of the oligonucleotide and the anchor sequence. One illustrative example is shown in Example 6 and FIG. 16A.

In some instances, the binders 800 are attached to the arms or the connector chains via Van der Waals forces, hydrogen binding, and/or electrostatic forces.

In other instances, locking molecules are used to attach the binders 800 to the arms or the connector chains. The locking molecule comprises a polynucleotide configured to bind to the binders 800. Such binding affinity provides for stability of the network of polynucleotides. In addition, for some immunotherapy mechanisms described herein that rely on separation of the binders 800 from the locking molecule, such binding affinity optimally is designed or selected to enable separation of the binders 800 and locking molecules upon binding of the binders 800 to the target antigens or biomarkers on the surface of the biological entity, e.g., in view of the binding avidity between binder and the target antigens or biomarkers.

### Spacing Between the Binders

The spacing between the uniquely addressable loci of antigen binders on the network of polynucleotides is designed based on the spacing of the antigens on the target analyte (e.g., the target cell) and dimensions of the antigens, such that the antigen binders are positioned on the network of polynucleotides in a predetermined two-dimensional or three-dimensional spatial pattern that matches a two-dimensional or three-dimensional spatial pattern of the antigens on the target analyte.

Similarly, the spacing between the uniquely addressable loci of the biomarker binders on the network of polynucleotides is determined based on the spacing of the biomarkers on the immune cells and also the dimensions of the biomarkers, such that the biomarker binders are positioned on the network of polynucleotides in a predetermined two-dimensional or three-dimensional spatial pattern that matches a two-dimensional or three-dimensional spatial pattern of the biomarkers on the target analyte (e.g., the target cell).

Although the following disclosure describes exemplary methods in determining the distances between antigens on target analytes, each method can also be used to determine the distances between biomarkers on immune cells.

In some embodiments, for target antigens that are known (for example, EGFR), information on a target antigen's attributes that are helpful for determining the distance between uniquely addressable loci of the binders on the network of polynucleotides is known and can be obtained from published literature (for example, EGFR). This information may include for example: 1) the dimension of the target antigen, 2) the target's predilection to cluster, and 3) the spacings between those clusters. An example of the former target's predilection to cluster, and the spacings between those clusters, as well as other information relevant to EGFR clustering and dimensional characteristics can be found in for EGFR by Wollman et al. (2022) J. R. Soc. Interfacel9: 20220088 (royalsocietypublishing.org/doi/epdf/10.1098/rsif2022.0088), Wang et al. (2021) (pubs.acs.org/doi/10.1021/nl3012227), Cahall et al. (2015) (journals.sagepub.com/doi/full/10.4137/BCBCR.525461?rfr_dat=cr_pub++0pubmed&url_ve r=Z39.88-2003&rfr_id=ori %3Arid %3Acrossreforg), and Zhang et al. (2015) (pubs.acs.org/doi/10.1021/acs.analchem.5b02572). An example of the latter can be found for DR5 by Wang et al. (2021) (pubs.acs.org/doi/10.1021/acsnano.OcI0104).

Designing Networks of Polynucleotides to Display Binders Spatially Aligned with Non-Clustered Antigens

In some embodiments, the inter-binder distance on the network of polynucleotides is equal to or greater than the widest dimension of the target antigen. In cases where the target antigen is both known to cluster and the spacings between those clusters are well elucidated, then networks of polynucleotides can be designed with patterns that match those of target antigen distribution on the target analyte.

In some cases, the structural information of the target antigen and its distribution on target analytes is unknown. In these cases, the 3D structure of the antigen can be predicted using computational methods (e.g., AlphaFold) and the dimensions of the predicted structure can be measured. In some cases, the structure information can be gleaned from cryoEM, NMR or other structural analysis methods.

In some embodiments, regardless of whether the structural information of the antigen is from a literature search or a computational prediction, the inter-binder distance of the network of polynucleotides can be optimized by designing a series of networks of polynucleotides attached with the same binders with the only difference between the networks being the inter-binder distances. The inter-binder distances can be designed, for example, to gradually increase among the different networks, starting from a distance equal to or greater than the widest dimension of the antigen. The networks of polynucleotides are then loaded with binders and the activities of the resultant biopolymers on target cells can be tested in cell-based assays, for example, cell-binding assays and/or cell-killing assays. Optimal inter-binder distances of the network for the target cells can be determined based on the inter-binder distances in the biopolymers that exhibit the desired cell-binding or cell-killing activity.

Designing Networks of Polynucleotides to Display Binders Spatially Aligned with Clustered Antigens

In some cases, the antigen forms clusters on the target analyte. Networks of polynucleotides can be designed such that antigen binders also form clusters in a predetermined two-dimensional or three-dimensional spatial pattern that matches a two-dimensional or three-dimensional spatial pattern of antigen clusters.

In some cases, the antigen clustering on cell surface is known and well characterized. For example, Wollman et al. (2022) found that EGFR forms clusters typically of 6 EGFRs at 5 nm spatial arrangements with an overall cluster diameter of approximately 20 nm. Clusters of binders can be displayed on a surface of a network of polynucleotides having an intra-cluster distance of 5 nm.

As one illustrative example, to design the individual cluster dimensions, the network of polynucleotides designer can take the square of the intra-cluster diameters (for roughly square clusters; for other Euclidean geometric shapes, such as circles, ovals, octagons, etc., the relevant area equations (E_{A}) can be used) and divide by ICDₐ to determine the intra-cluster binder arrangements. To determine inter-cluster binder distances (ICDₑ), additional information is required. Continuing with the EGFR example, a network of polynucleotides designer can estimate the surface area of a breast cancer cell (Cell_{SA}) at approximately 415 to 922 μm² (Cahall et al. (2015) (PMID: 26309407)), and the EGFR copy number (C_{N}) at approximately 200,000 per cell (Zhang et al. (2015) (PMID: 26368334)). At 6 EGFRs per cluster (Cl_{Na}), and 200,000 EGFR copies per cell, this translates to approximately 33,333 EGFR clusters per cell (Cl_{Ne}). With a cluster diameter (Cl_{D}) of approximately 20 nm, the individual area of a cluster (Cl_{A}) is approximately 400 nm². A per cluster surface area of 400 nm² multiplied by the number of clusters (33,333) equals 13,333,200 nm², which is the total cluster surface area (Cl_{T}), and which assumes no spacing between the clusters. Dividing the average cell surface area of a breast cancer cell (assume 668.5 μm²) by the EGFR total cluster surface area of 13,333,200 nm² yields 50 nm², which is the inter-cluster spacing coefficient (Cl_{K}) whose square root is an estimate of EGFR inter-cluster spacing (Cl_{Z}): 7 nm. Therefore, in this example, the network of polynucleotides design would arrange anti-EGFR binders into clusters with an intra-cluster distance of 5 nm, a cluster diameter of 20 nm, and an inter-cluster spacing of at least 10 nm (the next spatial distance possible on most network of polynucleotides designs after 5 nm).
ICDₐ: Intra-cluster binder distance
E_{A}: Area equation for relevant 2-dimensional Euclidean geometric shape
ICDₑ: Inter-cluster binder distance
Cell_{SA}: Surface area of the target cell(s)
C_{N}: Target antigen copy number on target cell(s)
Cl_{Na}: Target antigen copy number in clusters
Cl_{Ne}: Target antigen clusters per target cell(s)
Cl_{D}: Diameter per cluster
Cl_{A}: Area per cluster
Cl_{T}: Total surface area of all antigen clusters on the target cell(s), per target cell(s)
Cl_{K}: Inter-cluster spacing coefficient on the target cell(s)
Cl_{Z}: Inter-cluster spacing

### Formula Steps:

$$\begin{matrix}C {l}_{N E} = {C}_{N} / C {l}_{N A} \\ C {l}_{A} = {Cl}_{D}^{2} or {E}_{A} , as is relevant \\ C {l}_{T} = {Cl}_{A} \times C {l}_{N e} \\ C {l}_{K} = {Cell}_{S A} / C {l}_{T} \\ IC {D}_{e} = {Cl}_{K}^{0.5} \\ = {Cell}_{S A} / C {l}_{T} \\ = {Cell}_{S A} / ( C {l}_{A} \times C {l}_{N e} ) \\ = {Cell}_{S A} / ( ( C {l}_{D}^{2} or {E}_{A} ) ) \times ( {C}_{N} / C {l}_{N A} ) )\end{matrix}$$

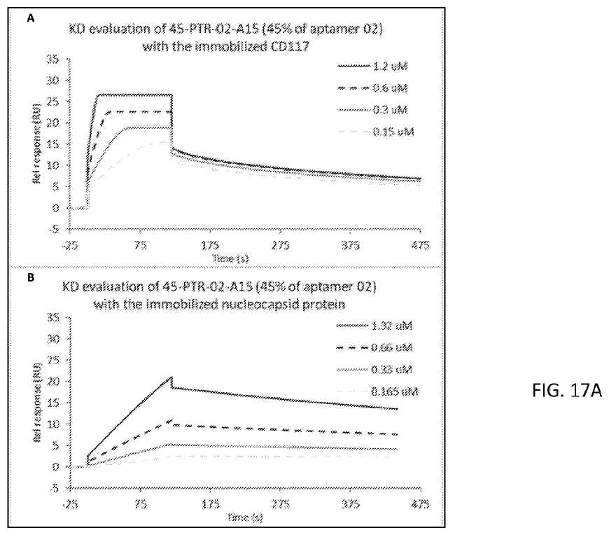

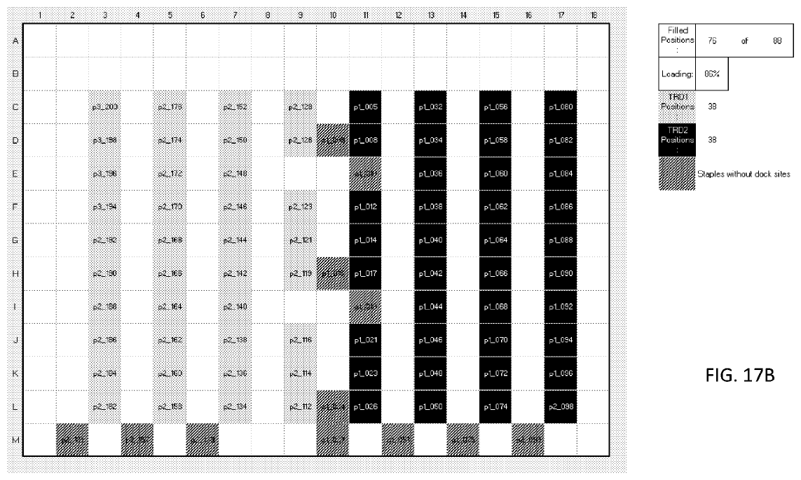

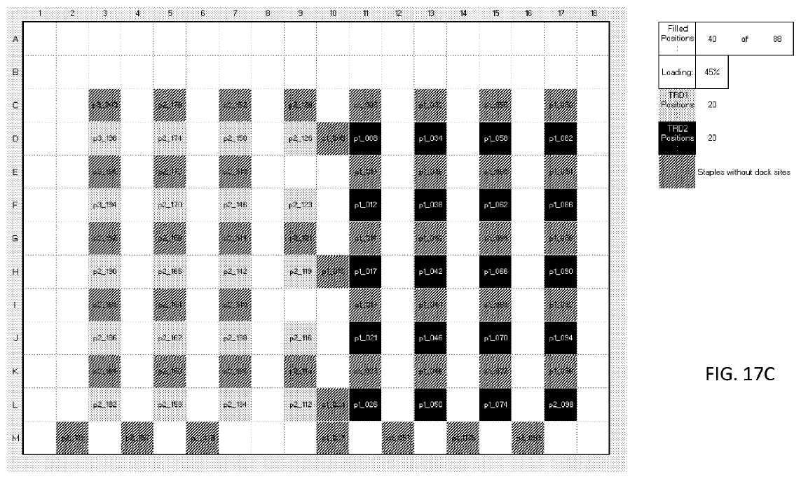

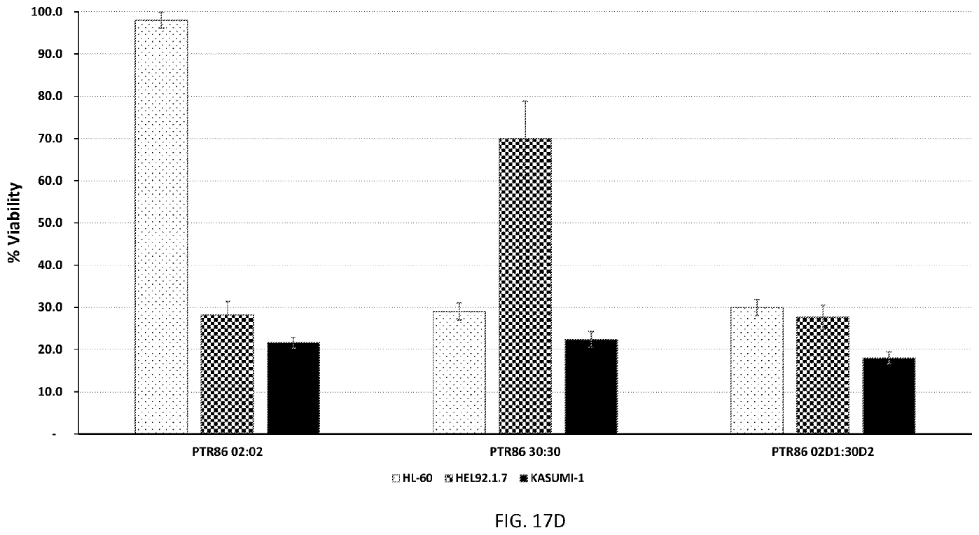

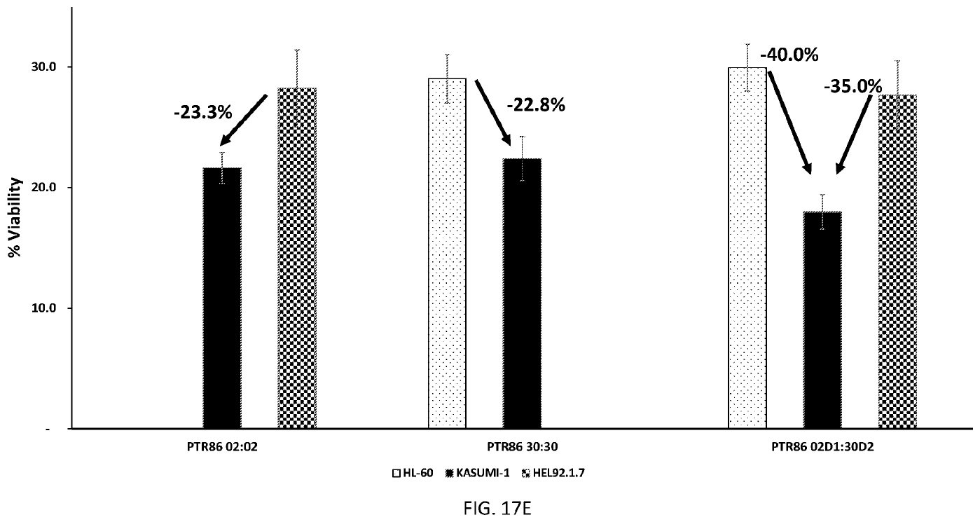

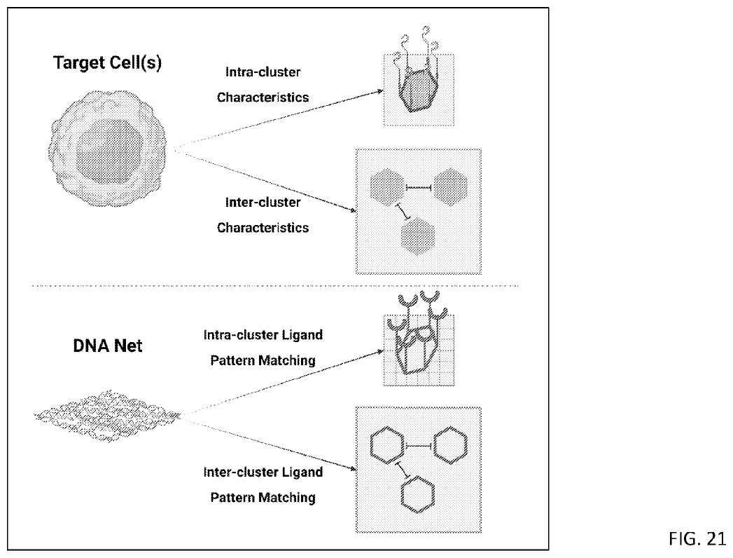

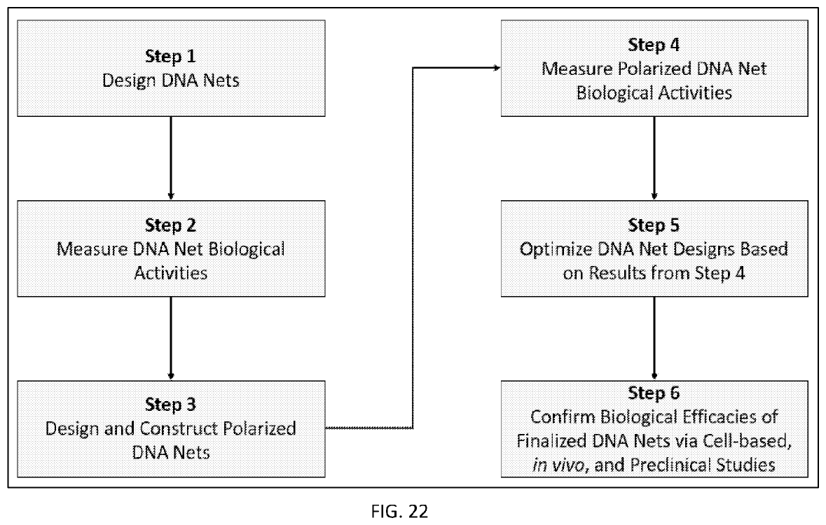

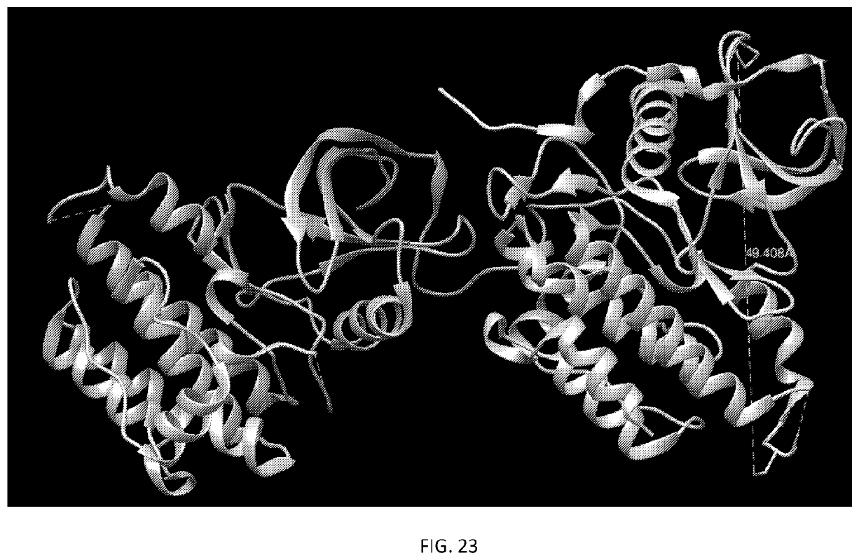

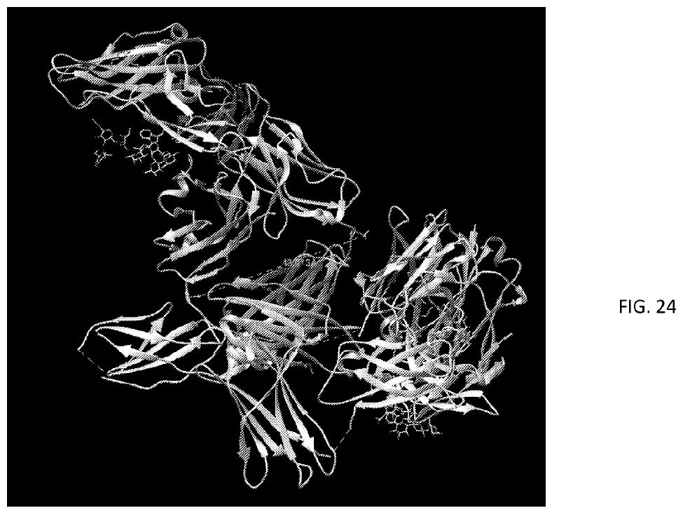

In some cases, the antigen clustering on the cell surface is unknown. In these cases, the network of polynucleotides inter-cluster design is empirical, generally following the workflow illustrated in FIG. 22. The first step is to design various networks of polynucleotides (e.g., 3x3 nets or TRD1 tiles, etc.) with binders arranged at the theoretically optimal intra-cluster binder distance, which is typically the widest dimension of the antigen molecule. Additional versions of networks of polynucleotides displaying the same binders but different binder densities can be designed. The biological activity of these networks of polynucleotides are confirmed by binding assays and/or biological activity studies. Binding assays report on the binding characteristics of different networks of polynucleotides and allow comparison of those binding characteristics. Biological activity studies report on the biological activities of different networks of polynucleotides and allow comparison of those biological activities. Those networks of polynucleotides with the optimal binding characteristics and/or biological activities, as determined by the designer and mandated by the application, are selected for further development. Upon elucidation of the optimal binder spacing, network of polynucleotides designs bearing clusters of binders with optimized inter-binder distances are constructed (Step 3), such as on the network of polynucleotides PTR86 described in FIG. 17B. These network of polynucleotides designs permit the tuning of inter-cluster spacings, whose binding efficacies are determined by binding assays and biological efficacy studies (Step 4). Step 5 involves the optimization of the network of polynucleotides designs in response to results from Step 4. The intended design outcome (e.g., a network of polynucleotides composed of clusters of the same binder, clusters of different binders but against the same target, clusters of different binders that bind different targets) determines the suite of network of polynucleotides prototypes designed, constructed, and tested via binding assays and biological efficacy studies. Ultimately, cell-based binding assays and biological efficacy studies will select the final network of polynucleotides candidate for the intended application for further cellular in vitro, in vivo, and preclinical characterization (Step 6).

In some embodiments, a network of polynucleotides has intra-cluster binder distances in the range of between 1 nm and 15 nm, between 2 nm and 12 nm, between 2 nm and 10 nm, between 1 nm and 8 nm, or between 2 nm and 6 nm.

In some embodiments, a network of polynucleotides has inter-cluster binder distances of the antigen binder clusters is in the range from 3 nm to 25 nm, from 4 nm to 22 nm, from 3 nm to 20 nm, from 4 nm to 18 nm, or from 5 nm to 16 nm.

As one illustrative example, in a 3x3v1 net (a DNA net), each polynucleotide junction has three binder docking sites forming a binder cluster. Within the cluster, the average spacing of the binders (intra-cluster binder distance) is 6 nm. The average spacing between cluster centers (inter-cluster binder distance), is 15 nm.

As another illustrative example, in a 3x3v2 net (a DNA net), each polynucleotide junction has one binder docking site. The average inter-binder distance spacing is 15 nm.

As another illustrative example, in a TRD1 (DNA origami), the average inter-binder distance is 5 nm.

### Densities and Ratios of Different Binders on the Same Biopolymer

In some embodiments, the binder density, i.e., the number of uniquely addressable loci that are docked with binders over the total number of available uniquely addressable loci, are controlled such that they can most efficiently capture the antigens on target analytes or biomarkers on immune cells. Typically, a locus is only ready for docking a binder if the anchor sequence (a sequence that can hybridize to the binder) is present at the locus. Thus, by adjusting the number of anchor sequences, the binder density can be adjusted.

For example, a DNA origami PTR can have 45% of the binder docking sites occupied with binder, and the resultant biopolymer is referred to as PTR45.

### Spatial Arrangement of Different Binders on the Same Biopolymer

In some embodiments, the binder spatial arrangement, i.e., the location of uniquely addressable loci that are docked with binders on a surface of the network of polynucleotides, are controlled such that they can most efficiently capture the antigens on target analytes or biomarkers on immune cells. Typically, a locus is only ready for docking a binder if the anchor sequence (a sequence that can hybridize to the binder) is present at the locus. Thus, by adjusting the locations of anchor sequences, the binder spatial arrangement can be adjusted.

For example, DNA origami PTR45 can have 45% of the binder docking sites occupied with binders, and the locations of the anchor sequences on a surface of PTR45 determines the spatial arrangement of the binders in patterns, such as, but not limited to, an evenly spaced grid across the network of polynucleotides surface, or clustered on one half of the network of polynucleotides surface, or in a cluster occupying the center of the network polynucleotides.

### Multiplexing

In some embodiments, antigen binders displayed on a network of polynucleotides bind to the same antigen. In some embodiments, antigen binders displayed on a network of polynucleotides bind to two, three, or more different antigens. In some embodiments, the biopolymer complex comprises a plurality of first antigen binders binding to a first antigen (e.g., CD117) and a plurality of second antigen binders binding to a second antigen (e.g., CD123), wherein the first and the second antigens are different.

In some embodiments, biomarker binders displayed on a network of polynucleotides bind to the same biomarker. In some embodiments, biomarker binders displayed on a network of polynucleotides bind to two, three, or more different biomarkers. In some embodiments, the biopolymer complex comprises a plurality of first biomarker binders binding to a first biomarker and a plurality of second antigen binders binding to a second biomarker wherein the first and the second biomarkers are different.

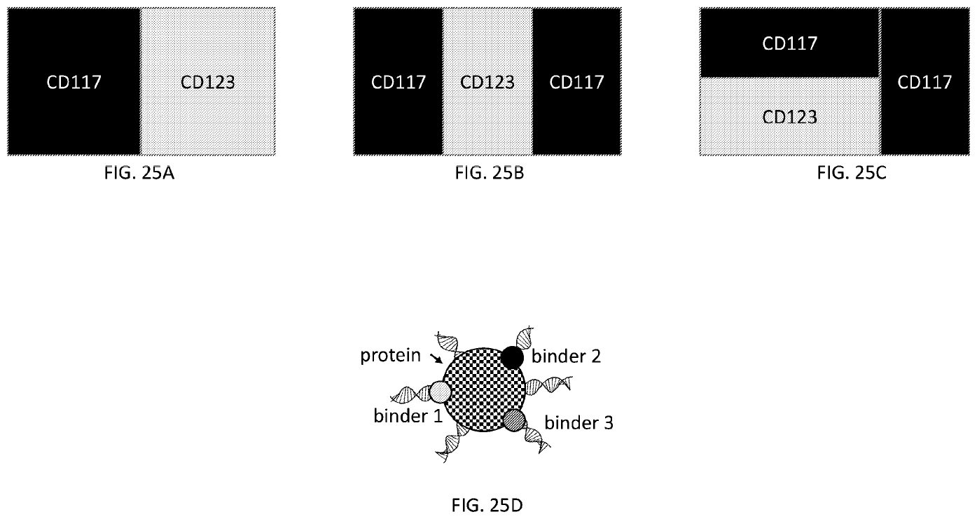

In some cases, target analytes express multiple antigens and the expression levels between multiple antigens vary depending on disease type and disease stage. A network of polynucleotides can be designed, for example, by changing the number of anchor sequences for different antigen binders, so that the ratio of the different antigen binders in the network of polynucleotides matches the ratio of the expression levels of different antigens for the disease to be treated. FIG. 25A-25C show illustrative examples of biopolymers on which different binders are attached and the ratio of the CD 117 binders to the CD123 binders are 1:1, 2:1, and 2:1, respectively. In some cases, a biopolymer with an equal number of CD117 binders and CD123 binders shows better killing activity on certain tumor cell lines, for example, the Kasumi AML leukemia cell lines, then a biopolymer in which the numbers of the two antigens are different.

### Additional Agents

In some instances, the artificial biopolymer complex further comprises one or more therapeutic agents attached to the network of polynucleotides. As shown in FIG. 9, in addition to recruiting the immune cells (e.g., white blood cells) for triggering immunotherapy, the artificial biopolymer complex 900 may also be designed to carry a therapeutic agent 905 via binder 910 binding to cell surface antigens 915 with subsequent internalization 920. Therapeutic agent means a drug, protein, peptide, gene, compound or other pharmaceutically active ingredient that can be used in the application of chemotherapy, antibody therapy, immunotherapy, immunization, or the like for the treatment or mitigation of a disease condition or ailment.

In some embodiments, the therapeutic agent is an intercalating agent. The intercalating agent can interact with DNA or DNA modifying enzymes to induce apoptosis of cells. Nonlimiting examples of intercalating agents include daunorubicin, Cisplatin, Fluoroquinolones, ciprofloxacine, and so on.

In some embodiments, the therapeutic agent is a non-intercalating chemotherapy agent that inhibits target cell proliferation or kills target cells (e.g., tumor cells). chemotherapy agents that can be used including, but not limited to, e.g., auristatin, duocarmycins, camptothecins, maytansinoids, pyrrolobenzodiazepines, ricin A chain, a maytansinoid, taxol, mitomycin, etoposide, tenoposide, vincristine, vinblastine, colchicine, dihydroxy anthracin dione, methotrexate, actinomycin, a diphtheria toxin, exotoxin A from Pseudomonas, Pseudomonas exotoxin40, abrin, abrin A chain, modeccin A chain, alpha sarcin, gelonin, mitogellin, restrictocin, cobran venom factor, a ribonuclease, engineered Shiga toxin, phenomycin, enomycin, curicin, crotin, calicheamicin, Saponaria officinalis inhibitor, glucocorticoid, auromycin, yttrium, bismuth, combrestatin, duocarmycins, dolastatin, or cc1065. In some embodiments, the therapeutic agent is a proliferation inhibitor, a lytic agent, a DNA or RNA synthesis inhibitor, a membrane permeability modifier, a DNA metabolite, a dichloroethylsulfide derivative, a protein production inhibitor, a ribosome inhibitor, or an inducer of apoptosis.

The therapeutic agent can be loaded to a network of polynucleotides in various ways. In one embodiment, the therapeutic agent (for example, a chemotherapy agent) is mixed with the binders (for example, the aptamers) first to allow the therapeutic agent diffuse and intercalate into the binders. Excessive molecules of the therapeutic agent are removed by, for example, size exclusion chromatography or filtration. The binders loaded with the therapeutic agent are then displayed on the network of polynucleotides. In some cases, as compared to mixing chemotherapy agents, the binders, and the network of polynucleotides in one mixture, the method of mixing a chemotherapy agent with the binders to form chemotherapy agent-loaded binders and then docking the chemotherapy agent-loaded binders onto the network of polynucleotides is less likely to cause precipitation of the network of polynucleotides and thus the loss of the chemotherapy agent.

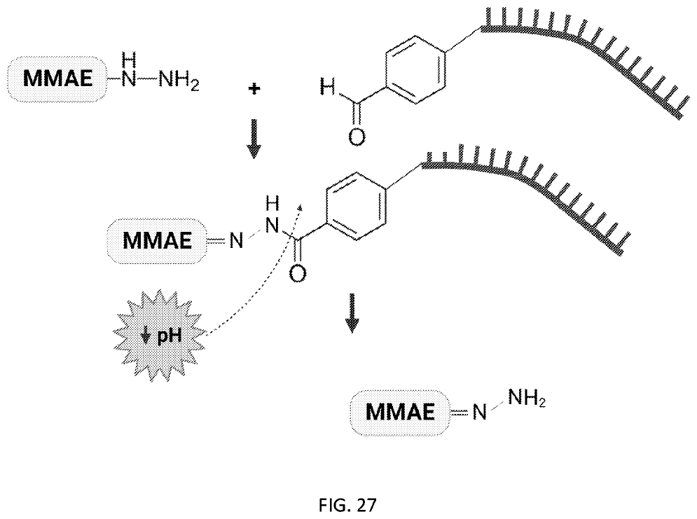

In some embodiments, the therapeutic agent is directly conjugated to the binders or the network of polynucleotides via, for example, a sulfo-SMCC linker or any other conjugation chemistry. As an illustrative example, the conjugation of a non-intercalating therapeutic agent, e.g., monomethyl auristatin E (MMAE), occurs via the formation of an acid-labile hydrazone between MMAE and a binder or binders on the network of polynucleotides, or to polynucleotides comprising the network of polynucleotides (FIG. 27). MMAE bearing a hydrazide is reacted with a binder or polynucleotide bearing a benzaldehyde to form the hydrazone. In some embodiments, upon internalization into a cell and, specifically, into a lysosome, the network of polynucleotides bearing MMAE experiences a low pH, which cleaves the hydrazone and releases the MMAE inside the cell.

In some embodiments, the therapeutic agent is siRNA. The siRNA can silence expression that is critical for cell growth, proliferation, or other pathological processes. Once the biopolymer is delivered to the target cell (e.g., a tumor cell) via the interaction of the binder in the biopolymer and the antigen on the target cells, the siRNA will exert its effect by decomposing into single strands and binding to their distinct target messenger RNA (mRNA) sequences. This action catalyzes a string of activities that promotes the degradation of target mRNA to break and degrade, further halting translation.

In some embodiments, the therapeutic agent is an immunoadjuvant, an antibody, a peptide, a protein domain, a nanobody, an scFv, a small molecule drug, a siRNA, a nanoparticle. In some embodiments, other biologics can also be used as the therapeutic agent, for example, a protein (other than antibody, peptide, protein domain, nanobody, or scFv), non-siRNA nucleic acid, lipid, chelator, sugar, glycoprotein, or proteoglycan.

In some embodiments, the network of polynucleotides is used as the backbone to develop a Nucleic Acid Scaffold Drug Conjugate (NASDC). NASDCs can exert the same, similar, or a superior therapeutic effect as antibody-drug conjugates (ADCs), which are a class of drugs whereby an antibody or antibodies are conjugate to a therapeutic agent, such as a cytotoxic warhead. Because the linker site on polynucleotides is addressable, the network of polynucleotides described in the invention can be rationally designed carrying a hybrid cargo of, but not limited to, intercalating agents (including daunorubicin, doxorubicin) via intercalation, non-intercalating therapeutic agents via covalent conjugation, or siRNAs via hybridization at a higher capacity attainable by ADCs. ADCs typically have a drug-to-antibody ratio (DAR) of 2 to 4 identical therapeutic agents per antibody (see citation 1 below). For comparison, one embodiment of the present invention has a network of polynucleotides capable of docking up to 96 targeting ligands. For intercalators such as daunorubicin, the drug-to-network (DNR) could be higher than that which can be attained by ADCs. Assuming the usage of 50 base pair intercalator-carrying oligos and 100% loading efficiency, on a network of polynucleotides with 96 total docking sites and 44 occupied by targeting ligands, then the daunorubicin DNR in this case would be 2,400—a stoichiometry that is theoretically impossible with current ADC technology. Combining the higher warhead load capacity, hybrid load of multiple therapeutic agents via addressable linkers and higher selectivity for tumor cell targeted delivery, the resulting NASDC greatly improve the therapeutic index of the drug.

In some embodiments, the network of polynucleotides can exert the same, similar, or a superior therapeutic effect as antibody-drug conjugates (ADCs), which are a class of drugs whereby an antibody or antibodies are conjugate to a therapeutic agent, such as a cytotoxic warhead. The network of polynucleotides described in the invention are capable of carrying both intercalating and non-intercalating therapeutic agents at stoichiometries the same, less than, and greater than those attainable by ADCs. ADCs typically have a drug-to-antibody ratio (DAR) of 2 to 4 therapeutic agents per antibody (M. R. Gordon et al., Bioconjug Chem, vol. 26, no. 11, pp. 2198-2215, 2015). There is ongoing work producing incremental improvements to ADC DAR, such as a recent report of 6 drugs per antibody (see C. M. Yamazaki et al., Nature Communications, vol. 12, no. 3528, 2021). For comparison, one embodiment of the present invention has a network of polynucleotides capable of docking 1 to 96 targeting ligands. In this example, the network of polynucleotides can attain a drug-to-network ratio (DNR) as high as 96. For intercalators such as daunorubicin, the DNR could be higher. Assuming the usage of 50 base pair intercalator-carrying oligos and 100% loading efficiency, on a network of polynucleotides with 96 total docking sites and 44 occupied by targeting ligands, then the daunorubicin DNR in this case would be 2,400—a stoichiometry that is theoretically impossible for current ADC technology to attain.

### Methods of Manufacturing Networks of Polynucleotides

Methods of manufacturing biopolymers are known and described in the International application publication WO2022109155A1 and United States patent U.S. Pat. No. 8,501,923B2, the entire disclosures of which are herein incorporated by reference.

For example, a network of polynucleotides in the form of DNA origami as shown in FIG. 2 can by produced as follows. Typically, the connector chains in the DNA origamis are shorter, typically 6-60 bases long (e.g., 10-30 bases long). Structural chains are longer, typically about 6000 to 9000 bases long. Generally, a connector chain is complementary to at least two regions of the structural chain. Generally, the connector chain has a region of at least 6 nucleotides that are complementary to corresponding regions of a structural chain. The entire connector chain will generally be at about 6 to 60 nucleotides in length. The complementary regions on the scaffold can be adjacent or not adjacent. In one embodiment, the connector chain is complementary to three regions of the structural chain. For example, a connector chain can be used that has regions complementary to three regions, and has complementary regions of 8, 16 and 8 nucleotides respectively with no intervening spacer nucleotides between the complementary regions. Along the edges or seams that occur in a shape or structure, some connector chains can be used that bind only a single region of the structural chain. Such connector chains aid in stiffening the shape or structure.

Once the connector chains are designed, they are synthesized, mixed with the structural chain in a buffer solution, heated (for example to about 90 degrees centigrade), and cooled to room temperature. The buffer solution is selected to allow for hybridization of the structural chain and connector chains. In one embodiment, the buffer comprises magnesium. Generally, a stoichiometric excess of the connector chains is used. In one embodiment, 2-100 times as many connector chains are present as would be needed to fold all the structural chains. Typically, the structures are folded in solution and applied to a substrate after they have been formed. Where the shape or structure is a two- or three-dimensional shape or structure, the solution of scaffold and connector chains is applied to a substrate after annealing.

The correct hybridization of connector chains and structural chains is assured by the design of their nucleotide sequences, which are designed with the consideration of conditions, such as—but not limited to—the temperatures and incubation times (i.e., thermal cycling), the concentrations of the constituent connector and structural chains in the mixture, the magnesium cation (Mg²⁺) and sodium cation (Na⁺) concentrations, and other variables capable of influencing hybridization. This process designs connector chains with primary sequences that will hybridize at targeted complementary sites on structural chains. These sites can be unique or ubiquitous. In some embodiments, unique complementary sites are designed such that specific connector chains will only be found at one specified location within the fully assembled network of polynucleotides. This property also enables the precise arrangement of docking sites that are extended from individual connector chains, creating binder docking sites (aka. loci) that are uniquely addressable. That is, each binder site is associated with a nucleotide sequence that is unique to that locus on the network of polynucleotides. This in turn enables the precise docking of binders at uniquely addressable docking sites. The docking of binders can be confirmed by various methods, such as—but not limited to—tapping mode Atomic Force Microscopy (AFM).

Networks of polynucleotides comprising Holliday junctions, such as the one shown in FIG. 6 can be manufactured in a similar fashion, except that connector chains are not used in the design or manufacture of DNA nets. DNA nets are assembled by the hybridization of multiple polynucleotide structural chains. This process designs structural chains with primary sequences that will hybridize at targeted complementary sites on complementary structural chains. These sites can be unique or ubiquitous. When unique complementary sites are designed, specific structural chains will only be found at one specified location within monomeric polynucleotide network structural units of fully assembled networks of polynucleotides. This property enables the precise arrangement of uniquely addressable docking sites within Holliday junctions formed by hybridized structural chains. This in turn enables the precise docking of binders at uniquely addressable individual docking sites. The docking of binders at individual sites can be confirmed by various methods, such as—but not limited to—tapping mode Atomic Force Microscopy (AFM).

### Methods of Treatment

Disclosed herein is a method of treating a subject with an artificial biopolymer complex described above.

Without being bound to a particular theory, an artificial biopolymer can be used to treat a subject in a number of ways. In some embodiments, an artificial biopolymer can itself exert a therapeutic due to the ability of the docked binders to immobilize cell surface antigens in a pattern that results in a treatment effect. In some embodiments, binding of the binders to the antigens on the surface of the target cell prevent formation of multimers or clusters of antigens when formation of multimers or clusters may result in disease conditions. In some embodiments, interactions of the binders in the biopolymer complex to antigens on the surface of a target cell may advantageously facilitate the formation of clusters of the antigens and induce apoptosis of the target cell. One example is DR5, when forming clusters on cells, directs the cells to undergo apoptosis. See, Wang et al. (2021) (PMID: 34019379).

In some embodiments, the binders on the artificial biopolymer bind the target cells and the therapeutic agent can exert an effect in the target cells.

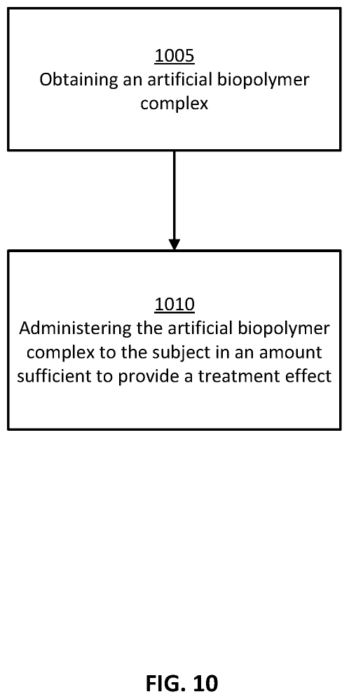

FIG. 10 illustrates an example of a process for treating a subject using an artificial biopolymer complex according to an embodiment of the present disclosure. The artificial biopolymer complex may be, for example, any of the artificial biopolymer complexes described with respect to FIGS. 1-9.

At block 1005, an artificial biopolymer complex is obtained. The artificial biopolymer complex may be obtained based on the desired type of immunotherapy to be provided for a given subject. For example, if a subject has leukemia such as AML then the artificial biopolymer complex obtained may have a dual-faced display of binders—the top surface displays 1-2 cluster(s) of CD3 binders to bind to T-cells (where CD3 is a TCR)—the bottom surface displays one or multiple binder clusters, each target one or more tumor specific antigens (for example, CD33, CD123, CD 117 are highly correlated to AML disease). Each binder cluster may have 6+ repeating binders forming a regular shape (hexagon or triangle) cluster where the inter-binder spacing is calculated using the dimension of these protein markers and their associated epitopes or domains. The tumor binding side of the complex may have a much stronger binding to the tumor cell, rather than to the immune cell because an immune cell will be unlikely to either express or express at high levels of all three markers. The multi-valency (density/distance of binders) allows for the artificial biopolymer complex to only bind tumor cells as opposed to immune cells, even if immune cells express some or all of the same biomarkers. The binding of the tumor cells further flattens the scaffold so that the CD3 binder cluster is exposed to recruit and enable T-cell killing. At block 1010, the artificial biopolymer complex is administered to the subject in an amount sufficient to provide a treatment effect. In some instances, the treatment effect is an acute, intermittent prophylactic, or prophylactic effect. The treatment effect is facilitated by binding of antigen binders to the antigens and/or the biomarker binders to the biomarkers. In some instances, the binding of the second set of binders to the biomarkers activates an immune response by immune cells (e.g., T-cells), administration of a biopolymer complex to a subject may induce one or more responses including: apoptosis by the target analyte, lysis by the target analyte, cellular differentiation by the target analyte, an activation of intracellular signaling pathways via phosphorylation of constituent kinases by the target analyte, a deactivation of intracellular signaling pathways via inhibition of the phosphorylation of constituent kinases, or dephosphorylation of constituent kinases by the target analyte, degranulation resulting in the release of secretory vesicle contents by the target analyte, the prevention of degranulation that would normally result in the release of secretory vesicle contents by the target analyte, induction of chemotaxis by the target analyte, prevention of chemotaxis by the target analyte, prevention of the transcription of a gene or genes by the target analyte, induction of the transcription of a gene or genes by the target analyte; prevention of the translation of a gene or genes by the target analyte; induction of the translation of a gene or genes by the target analyte.

In some instances, the binding of the first set of binders to the antigens activates a response by the target analyte to the artificial biopolymer complex. The response may be a cellular internalization of the artificial biopolymer complex by the target analyte. The cellular internalization of the artificial biopolymer complex by the target analyte may cause the release of the one or more therapeutic agents from the network of polynucleotides.

In some instances, the binding of the first set of binders to the antigens (e.g., antigens on tumor cells) causes the curvature of the network of polynucleotides to flatten, which facilitates the binding of the second set of binders to the biomarkers to the immune cells. In other instances, the binding of the first set of binders to the antigens causes the network of polynucleotides to curve, which blocks or facilitates the binding of the second set of binders to the biomarkers to immune cells. These curvatures can be determined and designed theoretically to yield particular shapes and curvatures with particular radii. The fact that the helical path of a double-stranded DNA composing the polynucleotide network (often referred to as “cylinders”) rotate 240° every 7 bp assuming that the DNA is in B-form with a twist density of 10.5 bp/tum (typically, B-form DNA can assume an over-twist and under-twist of 10.2 to 11 bp/turn) can be leveraged to create these curvatures. In particular, this is a property derived from the ability of DNA to behave as an elastically twistable rod, with a torsional modulus of ˜2.4-4.5 10¹⁹ erg·cm (1 erg=0.1 mJ), allowing dsDNA to have a curvature radius of 2 to 20 nm. In certain instances, this property can be used to create polynucleotide networks with varying curvatures by designing crossover planes for the connector chains also known as “staple” strands at various locations. Across polynucleotide networks, the connector chain locations influence helical twisting, twist density, and curvature. Those skilled in the art would understand that in silico tools and techniques (e.g., a tool called caDNAno) for designing 2D crossover planes and unique connector chains could be used to create the curvatures. In 2D structures, staples span adjacent cylinders. When designing a 3D structure, connector chains can span adjacent or distant cylinders. Typically, curvatures are measured in silico, such as in the aforementioned caDNAno software. Experimentally, methods such as, but not limited to, transmission electron microscopy (TEM) can be used to image and measure polynucleotide network angles and curvatures.

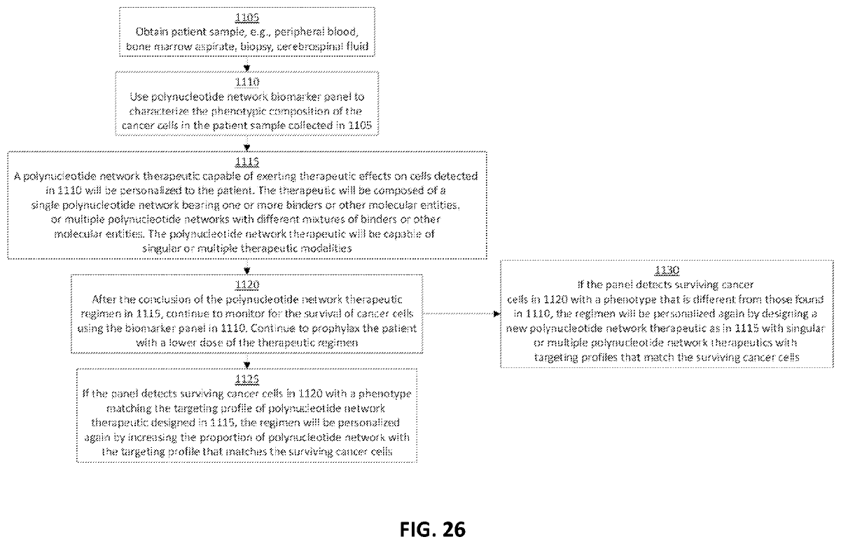

FIG. 26 illustrates an example of a process for treating a subject using an artificial biopolymer complex according to an embodiment of the present disclosure. The artificial biopolymer complex may be any of the artificial biopolymer complexes described above, for example, those with respect to FIGS. 1-9.

At block 1105, a subject sample, e.g., peripheral blood, bone marrow aspirate, biopsy, cerebrospinal fluid is obtained.

At block 1110, a polynucleotide network biomarker panel is used to characterize the phenotypic composition of the cancer cells in the subject sample collected in 1105.

At block 1115, a polynucleotide network capable of exerting therapeutic effects on cells detected in 1110 are designed personal to the subject. The therapeutic may be comprised of a single polynucleotide network bearing one or more binders or other molecular entities, or multiple polynucleotide networks with different mixtures of binders or other molecular entities. The polynucleotide network therapeutic may be capable of singular or multiple therapeutic modalities.

At block 1120, after the conclusion of the polynucleotide network therapeutic regimen in 1115, continue to monitor for the survival of cancer cells using the biomarker panel in 1110. In some instances, continue to prophylax the patient with a lower dose of the therapeutic regimen.

At block 1125, if the panel detects surviving cancer cells in 1120 with a phenotype matching the targeting profile of polynucleotide network therapeutic designed in 1115, the regimen may be personalized again by increasing the proportion of polynucleotide networks with the targeting profile that matches the surviving cancer cells.

At block 1130, if the panel detects surviving cancer cells in 1120 with a phenotype that is different from those found in 1110, the regimen will be personalized again by designing a new polynucleotide network therapeutic as in 1115 with singular or multiple polynucleotide network therapeutics with targeting profiles that match the surviving cancer cells.

### EXEMPLARY EMBODIMENTS

This disclosure includes the following exemplary embodiments.

1. An artificial biopolymer complex comprising a network of polynucleotides comprising structural units connected to one another via a series of arms and junctions,
wherein intersections of two or more arms form the junctions; a first set of binders attached to a first surface of the network of polynucleotides,
wherein: the first set of binders bind to antigens of a target analyte; and the first set of binders are attached at uniquely addressable loci on the arms forming the junctions, wherein the uniquely addressable loci are separated by predetermined inter-binder distances such that the first set of binders are positioned on the network of polynucleotides in a predetermined two-dimensional or three-dimensional spatial pattern that matches a two-dimensional or three-dimensional spatial pattern of the antigens on the target analyte; and a second set of binders attached to a second surface of the network of polynucleotides,
wherein: the second set of binders bind to biomarkers of immune cells; and the second set of binders are attached at uniquely addressable loci on the arms forming the junctions, wherein the uniquely addressable loci are separated by predetermined inter-binder distances such that the second set of binders are positioned on the network of polynucleotides in a predetermined two-dimensional or three-dimensional spatial pattern that matches a two-dimensional or three-dimensional spatial pattern of the biomarkers on the immune cells.

2. The artificial biopolymer complex of embodiment 1, wherein:
each of the structural units have a predetermined shape defined by one or more strands of polynucleotides; at least a portion of the one or more strands of polynucleotides of each structural unit is complementary to at least a portion of the one or more strands of polynucleotides of another structural unit, and the complementary portions of the strands of the polynucleotides are hybridized to connect the structural units;
the complementary portions of the strands of the polynucleotides form the arms with a predetermined length; and the intersections of the two or more arms form the junctions at a predetermined distance from one another based on the predetermined length of the arms.

3. The artificial biopolymer complex of embodiment 1, wherein the two-dimensional or three-dimensional spatial pattern of the antigens is defined by intermolecular spacing of the antigens on a surface of the target analyte.

4. The artificial biopolymer complex of embodiment 3, wherein the predetermined inter-binder distances of the uniquely addressable loci of the first set of binders match the intermolecular spacing of the antigens such that the first set of binders on the first surface of the network of polynucleotides align spatially with the antigens on the surface of the target analyte.

5. The artificial biopolymer complex of embodiment 3, wherein: each of the antigens is (i) a length and width in angstroms or nanometers from other antigens on the target analyte or (ii) a length, width, and depth from the other antigens on the target analyte, which define the intermolecular spacing of the antigens; and
each of the binders of the first set of binders is (i) a length and width in angstroms or nanometers from other binders of the first set of binders on the network of polynucleotides or (ii) a length, width, and depth in angstroms or nanometers from the other binders of the first set of binders on the network of polynucleotides, which defines the predetermined inter-binder distances of the uniquely addressable loci of the first set of binders; and the predetermined inter-binder distances of the uniquely addressable loci of the first set of binders match the intermolecular spacing of the antigens such that the first set of binders on the first surface of the network of polynucleotides align spatially with the antigens on the surface of the target analyte.

6. The artificial biopolymer complex of embodiment 1, wherein the two-dimensional or three-dimensional spatial pattern of the biomarkers is defined by intermolecular spacing of the biomarkers on a surface of the immune cells.

7. The artificial biopolymer complex of embodiment 6, wherein the predetermined inter-binder distances of the uniquely addressable loci of the second set of binders match the intermolecular spacing of the biomarkers such that the second set of binders on the second surface of the network of polynucleotides align spatially with the biomarkers on the surface of the immune cells.

8. The artificial biopolymer complex of embodiment 6, wherein: each of the biomarkers is (i) a length and width in angstroms or nanometers from other biomarkers on the immune cells or (ii) a length, width, and depth from the other biomarkers on the immune cells, which define the intermolecular spacing of the biomarkers; and each of the binders of the second set of binders is (i) a length and width in angstroms or nanometers from other binders of the second set of binders on the network of polynucleotides or (ii) a length, width, and depth in angstroms or nanometers from the other binders of the second set of binders on the network of polynucleotides, which defines the predetermined inter-binder distances of the uniquely addressable loci of the second set of binders; and the predetermined inter-binder distances of the uniquely addressable loci of the second set of binders match the intermolecular spacing of the biomarkers such that the second set of binders on the second surface of the network of polynucleotides align spatially with the biomarkers on the surface of the immune cells.

9. The artificial biopolymer complex of embodiment 1, wherein: each of the antigens comprises one or more epitopes; the first set of binders are arranged in sets of clustered antigen binders; each binder of a set of clustered antigen binders is attached to one of the two or more arms that form a junction; and the binders of each of the sets of clustered antigen binders are attached to the arms at uniquely addressable loci that are a predetermined distance from the junction, wherein the uniquely addressable loci are separated by predetermined intra-binder distances such that each set of clustered antigen binders are positioned on the network of polynucleotides in a predetermined two-dimensional or three-dimensional spatial pattern that matches a two-dimensional or three-dimensional spatial pattern of the one or more epitopes on an antigen.

10. The artificial biopolymer complex of embodiment 9, wherein the two-dimensional or three-dimensional spatial pattern of the one or more epitopes is defined by intramolecular spacing of the one or more epitopes on a surface of the antigen.

11. The artificial biopolymer complex of embodiment 10, wherein the predetermined intra-binder distances of the uniquely addressable loci of the binders of each of the sets of clustered antigen binders match the intramolecular spacing of the one or more epitopes such that the sets of clustered antigen binders on the first surface of the network of polynucleotides align spatially with the epitopes on the surface of the antigens.

12. The artificial biopolymer complex of embodiment 10, wherein: each of the one or more epitopes is (i) a length and width in angstroms or nanometers from other epitopes of the one or more epitopes on the antigen or (ii) a length, width, and depth from the other epitopes of the one or more epitopes on the antigen, which define the intramolecular spacing of the one or more epitopes; and each of the binders of each of the sets of clustered antigen binders is (i) a length and width in angstroms or nanometers from other binders of each of the sets of clustered antigen binders on the network of polynucleotides or (ii) a length, width, and depth in angstroms or nanometers from the other binders of each of the sets of clustered antigen binders on the network of polynucleotides, which defines the predetermined intra-binder distances of the uniquely addressable loci of the binders of each of the sets of clustered antigen binders; and the predetermined intra-binder distances of the uniquely addressable loci of the binders of each of the sets of clustered antigen binders match the intermolecular spacing of the epitopes such that each of the sets of clustered antigen binders on the first surface of the network of polynucleotides align spatially with the epitopes on the surface of the antigens.

13. The artificial biopolymer complex of embodiment 11 or 12, wherein the predetermined intra-binder distances of the uniquely addressable loci of the binders of each of the sets of clustered antigen binders is between 1 nm and 15 nm.

14. The artificial biopolymer complex of embodiment 1, wherein: each of the biomarkers comprises one or more epitopes; the second set of binders are arranged in sets of clustered biomarker binders; each binder of a set of clustered biomarker binders is attached to one of the two or more arms that form a junction; and the binders of each of the sets of clustered biomarker binders are attached to the arms at uniquely addressable loci that are a predetermined distance from the junction, wherein the uniquely addressable loci are separated by predetermined intra-binder distances such that each set of clustered biomarker binders are positioned on the network of polynucleotides in a predetermined two-dimensional or three-dimensional spatial pattern that matches a two-dimensional or three-dimensional spatial pattern of the one or more epitopes on a biomarker.

15. The artificial biopolymer complex of embodiment 14, wherein the two-dimensional or three-dimensional spatial pattern of the one or more epitopes is defined by intramolecular spacing of the one or more epitopes on a surface of the biomarker.

16. The artificial biopolymer complex of embodiment 15, wherein the predetermined intra-binder distances of the uniquely addressable loci of the binders of each of the sets of clustered biomarker binders match the intramolecular spacing of the one or more epitopes such that the sets of clustered biomarker binders on the second surface of the network of polynucleotides align spatially with the epitopes on the surface of the biomarkers.

17. The artificial biopolymer complex of embodiment 15, wherein: each of the one or more epitopes is (i) a length and width in angstroms or nanometers from other epitopes of the one or more epitopes on the biomarker or (ii) a length, width, and depth from the other epitopes of the one or more epitopes on the biomarker, which define the intramolecular spacing of the one or more epitopes; and each of the binders of each of the sets of clustered biomarker binders is (i) a length and width in angstroms or nanometers from other binders of each of the sets of clustered biomarker binders on the network of polynucleotides or (ii) a length, width, and depth in angstroms or nanometers from the other binders of each of the sets of clustered biomarker binders on the network of polynucleotides, which defines the predetermined intra-binder distances of the uniquely addressable loci of the binders of each of the sets of clustered biomarker binders; and the predetermined inter-binder distances of the uniquely addressable loci of the binders of each of the sets of clustered biomarker binders match the intermolecular spacing of the epitopes such that each of the sets of clustered biomarker binders on the second surface of the network of polynucleotides align spatially with the epitopes on the surface of the biomarkers.

18. The artificial biopolymer complex of embodiment 16 or 17, wherein the predetermined intra-binder distances of the uniquely addressable loci of the binders of each of the sets of clustered biomarker binders is between 1 nm and 15 nm.

19. The artificial biopolymer complex of any one of embodiments 1-18, further comprising one or more therapeutic agents attached to the network of polynucleotides.

20. The artificial biopolymer complex of any one of embodiments 1-19, wherein the network of polynucleotides has a curvature such that the second set of binders are at least partially enveloped by the second surface of the network of polynucleotides.

21. The artificial biopolymer complex of any one of embodiments 1-20, wherein the network of polynucleotides has been cross-linked to stabilize the structure and increase a half-life of a therapeutic agent.

22. The artificial biopolymer complex of any one of embodiments 1-21, wherein the network of polynucleotides has been modified with polymers, peptides, proteins, lipids, or a combination hereof to modulate pharmacokinetics and distribution in vivo.

23. An artificial biopolymer complex comprising: a network of polynucleotides comprising connector chains of polynucleotides attached to a structural chain of polynucleotides, wherein the connector chains are shorter than the structural chain; a first set of binders attached to a first surface of the network of polynucleotides, wherein:
the first set of binders bind to antigens of a target analyte; and the first set of binders are attached at uniquely addressable loci on the structural chain via the connector chains, wherein the uniquely addressable loci are separated by predetermined inter-binder distances such that the first set of binders are positioned on the network of polynucleotides in a predetermined two-dimensional or three-dimensional spatial pattern that matches a two-dimensional or three-dimensional spatial pattern of the antigens on the target analyte; and a second set of binders attached to a second surface of the network of polynucleotides, wherein: the second set of binders bind to biomarkers of immune cells; and
the second set of binders are attached at uniquely addressable loci on the structural chain via the connector chains, wherein the uniquely addressable loci are separated by predetermined inter-binder distances such that the second set of binders are positioned on the network of polynucleotides in a predetermined two-dimensional or three-dimensional spatial pattern that matches a two-dimensional or three-dimensional spatial pattern of the biomarkers on the immune cells.

24. The artificial biopolymer complex of embodiment 23, wherein at least a portion of each chain of the connector chains is complementary to at least a portion of the structural chain, and the complementary portions of the connector chains and structural chain are hybridized to connect the connector chains to the structural chain.

25. The artificial biopolymer complex of embodiment 23, wherein the two-dimensional or three-dimensional spatial pattern of the antigens is defined by intermolecular spacing of the antigens on a surface of the target analyte.

26. The artificial biopolymer complex of embodiment 25, wherein the predetermined inter-binder distances of the uniquely addressable loci of the first set of binders match the intermolecular spacing of the antigens such that the first set of binders on the first surface of the network of polynucleotides align spatially with the antigens on the surface of the target analyte.

27. The artificial biopolymer complex of embodiment 25, wherein:
each of the antigens is (i) a length and width in angstroms or nanometers from other antigens on the target analyte or (ii) a length, width, and depth from the other antigens on the target analyte, which define the intermolecular spacing of the antigens; and
each of the binders of the first set of binders is (i) a length and width in angstroms or nanometers from other binders of the first set of binders on the network of polynucleotides or (ii) a length, width, and depth in angstroms or nanometers from the other binders of the first set of binders on the network of polynucleotides, which defines the predetermined inter-binder distances of the uniquely addressable loci of the first set of binders; and
the predetermined inter-binder distances of the uniquely addressable loci of the first set of binders match the intermolecular spacing of the antigens such that the first set of binders on the first surface of the network of polynucleotides align spatially with the antigens on the surface of the target analyte.

28. The artificial biopolymer complex of embodiment 23, wherein the two-dimensional or three-dimensional spatial pattern of the biomarkers is defined by intermolecular spacing of the biomarkers on a surface of the immune cells.

29. The artificial biopolymer complex of embodiment 28, wherein the predetermined inter-binder distances of the uniquely addressable loci of the second set of binders match the intermolecular spacing of the biomarkers such that the second set of binders on the second surface of the network of polynucleotides align spatially with the biomarkers on the surface of the immune cells.

30. The artificial biopolymer complex of embodiment 28, wherein:
each of the biomarkers is (i) a length and width in angstroms or nanometers from other biomarkers on the immune cells or (ii) a length, width, and depth from the other biomarkers on the immune cells, which define the intermolecular spacing of the biomarkers; and
each of the binders of the second set of binders is (i) a length and width in angstroms or nanometers from other binders of the second set of binders on the network of polynucleotides or (ii) a length, width, and depth in angstroms or nanometers from the other binders of the second set of binders on the network of polynucleotides, which defines the predetermined inter-binder distances of the uniquely addressable loci of the second set of binders; and
the predetermined inter-binder distances of the uniquely addressable loci of the second set of binders match the intermolecular spacing of the biomarkers such that the second set of binders on the second surface of the network of polynucleotides align spatially with the biomarkers on the surface of the immune cells.

31. The artificial biopolymer complex of embodiment 23, wherein:
each of the antigens comprises one or more epitopes;
the first set of binders are arranged in sets of clustered antigen binders; and
the binders of each of the sets of clustered antigen binders are attached at uniquely addressable loci on the structural chain via the connector chains, wherein the uniquely addressable loci are separated by predetermined intra-binder distances such that each set of clustered antigen binders are positioned on the network of polynucleotides in a predetermined two-dimensional or three-dimensional spatial pattern that matches a two-dimensional or three-dimensional spatial pattern of the one or more epitopes on an antigen.

32. The artificial biopolymer complex of embodiment 31, wherein the two-dimensional or three-dimensional spatial pattern of the one or more epitopes is defined by intramolecular spacing of the one or more epitopes on a surface of the antigen.

33. The artificial biopolymer complex of embodiment 32, wherein the predetermined intra-binder distances of the uniquely addressable loci of the binders of each of the sets of clustered antigen binders match the intramolecular spacing of the one or more epitopes such that the sets of clustered antigen binders on the first surface of the network of polynucleotides align spatially with the epitopes on the surface of the antigens.

34. The artificial biopolymer complex of embodiment 32, wherein:
each of the one or more epitopes is (i) a length and width in angstroms or nanometers from other epitopes of the one or more epitopes on the antigen or (ii) a length, width, and depth from the other epitopes of the one or more epitopes on the antigen, which define the intramolecular spacing of the one or more epitopes; and
each of the binders of each of the sets of clustered antigen binders is (i) a length and width in angstroms or nanometers from other binders of each of the sets of clustered antigen binders on the network of polynucleotides or (ii) a length, width, and depth in angstroms or nanometers from the other binders of each of the sets of clustered antigen binders on the network of polynucleotides, which defines the predetermined intra-binder distances of the uniquely addressable loci of the binders of each of the sets of clustered antigen binders; and
the predetermined intra-binder distances of the uniquely addressable loci of the binders of each of the sets of clustered antigen binders match the intermolecular spacing of the epitopes such that each of the sets of clustered antigen binders on the first surface of the network of polynucleotides align spatially with the epitopes on the surface of the antigens.

35. The artificial biopolymer complex of embodiment 33 or 34, wherein the predetermined intra-binder distances of the uniquely addressable loci of the binders of each of the sets of clustered antigen binders is between 1 nm and 15 nm.

36. The artificial biopolymer complex of embodiment 23, wherein:
each of the biomarkers comprises one or more epitopes;
the second set of binders are arranged in sets of clustered biomarker binders; and
the binders of each of the sets of clustered biomarker binders are attached at uniquely addressable loci on the structural chain via the connector chains, wherein the uniquely addressable loci are separated by predetermined intra-binder distances such that each set of clustered biomarker binders are positioned on the network of polynucleotides in a predetermined two-dimensional or three-dimensional spatial pattern that matches a two-dimensional or three-dimensional spatial pattern of the one or more epitopes on a biomarker.

37. The artificial biopolymer complex of embodiment 36, wherein the two-dimensional or three-dimensional spatial pattern of the one or more epitopes is defined by intramolecular spacing of the one or more epitopes on a surface of the biomarker.

38. The artificial biopolymer complex of embodiment 37, wherein the predetermined intra-binder distances of the uniquely addressable loci of the binders of each of the sets of clustered biomarker binders match the intramolecular spacing of the one or more epitopes such that the sets of clustered biomarker binders on the second surface of the network of polynucleotides align spatially with the epitopes on the surface of the biomarkers.

39. The artificial biopolymer complex of embodiment 37, wherein:
each of the one or more epitopes is (i) a length and width in angstroms or nanometers from other epitopes of the one or more epitopes on the biomarker or (ii) a length, width, and depth from the other epitopes of the one or more epitopes on the biomarker, which define the intramolecular spacing of the one or more epitopes; and
each of the binders of each of the sets of clustered biomarker binders is (i) a length and width in angstroms or nanometers from other binders of each of the sets of clustered biomarker binders on the network of polynucleotides or (ii) a length, width, and depth in angstroms or nanometers from the other binders of each of the sets of clustered biomarker binders on the network of polynucleotides, which defines the predetermined intra-binder distances of the uniquely addressable loci of the binders of each of the sets of clustered biomarker binders; and
the predetermined inter-binder distances of the uniquely addressable loci of the binders of each of the sets of clustered biomarker binders match the intermolecular spacing of the epitopes such that each of the sets of clustered biomarker binders on the second surface of the network of polynucleotides align spatially with the epitopes on the surface of the biomarkers.

40. The artificial biopolymer complex of embodiment 38 or 39, wherein the predetermined intra-binder distances of the uniquely addressable loci of the binders of each of the sets of clustered biomarker binders is between 1 nm and 15 nm.

41. The artificial biopolymer complex of any one of embodiments 23-40, further comprising one or more therapeutic agents attached to the network of polynucleotides.

42. The artificial biopolymer complex of any one of embodiments 23-41, wherein the network of polynucleotides has a curvature such that the second set of binders are at least partially enveloped by the second surface of the network of polynucleotides.

43. The artificial biopolymer complex of any one of embodiments 23-42, wherein the network of polynucleotides has been cross-linked to stabilize the structure and increase a half-life of a therapeutic agent.

44. The artificial biopolymer complex of any one of embodiments 23-43, wherein the network of polynucleotides has been modified with polymers, peptides, proteins, lipids, or a combination hereof to modulate pharmacokinetics and distribution in vivo.

45. A method for treating a subject, the method comprising:
obtaining the artificial biopolymer complex of any one of embodiments 1-40; and
administering the artificial biopolymer complex to the subject in an amount sufficient to provide a treatment effect.

46. The method of embodiment 45, wherein the treatment effect is a prophylactic effect or a therapeutic effect.

47. The method of embodiment 45, wherein the treatment effect is facilitated by binding of the first set of binders to the antigens and the second set of binders to the biomarkers.

48. The method of embodiment 47, wherein the binding of the second set of binders to the biomarkers activates an immune response by the immune cells.

49. The method of embodiment 47, wherein the binding of the first set of binders to the antigens activates a response by the target analyte to the artificial biopolymer complex.

50. The method of embodiment 48, wherein the response is a cellular internalization of the artificial biopolymer complex by the target analyte.

51. The method of embodiment 50, wherein the cellular internalization of the artificial biopolymer complex by the target analyte causes the release of the one or more therapeutic agents from the network of polynucleotides.

52. The method of embodiment 47, wherein the binding of the first set of binders to the antigens causes the curvature of the network of polynucleotides to flatten, which facilitates the binding of the second set of binders to the biomarkers.

53. The method of embodiment 45, wherein the binding of the first set of binders to the antigens causes the network of polynucleotides to curve, which blocks or facilitates the binding of the second set of binders to the biomarkers.

Embodiment A1 is an artificial biopolymer complex comprising: a network of polynucleotides comprising structural units connected to one another via a series of arms and junctions, wherein intersections of two or more arms form the junctions; antigen binders attached to a surface of the network of polynucleotides, wherein: the antigen binders bind to antigens of a target analyte; and wherein at least some of the antigen binders are attached at uniquely addressable loci on the arms forming the junctions, wherein the uniquely addressable loci are separated by predetermined inter-binder distances such that the antigen binders are positioned on the network of polynucleotides in a predetermined two-dimensional or three-dimensional spatial pattern that matches a two-dimensional or three-dimensional spatial pattern of the antigens on the target analyte.

Embodiment A2 is the artificial biopolymer complex of embodiment(s) A1, wherein: each of the structural units has a predetermined shape defined by one or more strands of polynucleotides; at least a portion of the one or more strands of polynucleotides of each structural unit is complementary to at least a portion of the one or more strands of polynucleotides of another structural unit, and the complementary portions of the strands of the polynucleotides of adjacent structural units are hybridized to connect the adjacent structural units; the complementary portions of the strands of the polynucleotides of adjacent structural units form the arms with a predetermined length; and the intersections of the two or more arms form the junctions at a predetermined distance from one another based on the predetermined length of the arms, wherein the predetermined distance is in the range of 5 nm to 999 nm.

Embodiment A3 is the artificial biopolymer complex of embodiment(s) A1 or 2, wherein each structural unit is defined by three polynucleotides.

Embodiment A4 is the artificial biopolymer complex of embodiment(s) A1, wherein the two-dimensional or three-dimensional spatial pattern of the binders is defined by intermolecular spacing of the antigens on a surface of the target analyte, and wherein the predetermined inter-binder distances of the uniquely addressable loci of the antigen binders match the intermolecular spacing of the antigens such that the antigen binders on the network of polynucleotides align spatially with the antigens on the surface of the target analyte.

Embodiment A5 is the artificial biopolymer complex of embodiment(s) A4, wherein the predetermined inter-binder distances of the uniquely addressable loci of the antigen binders match the intermolecular spacing of the antigens such that the antigen binders on the surface of the network of polynucleotides align spatially with the antigens on the surface of the target analyte.

Embodiment A6 is the artificial biopolymer complex of embodiment(s) A1, wherein: each of the antigens is (i) a length and width in angstroms or nanometers from other antigens on the target analyte or (ii) a length, width, and depth from the other antigens on the target analyte, which define intermolecular spacing of the antigens; and each antigen binder is (i) a length and width in angstroms or nanometers from other antigen binders on the network of polynucleotides or (ii) a length, width, and depth in angstroms or nanometers from other antigen binders on the network of polynucleotides, which defines the predetermined inter-binder distances of the uniquely addressable loci of antigen binders; and the predetermined inter-binder distances of the uniquely addressable loci of the antigen binders match the intermolecular spacing of the antigens such that the antigen binders on the first surface of the network of polynucleotides align spatially with the antigens on the surface of the target analyte.

Embodiment A7 is the artificial biopolymer complex of embodiment(s) A1, wherein: the antigens form antigen clusters on the target analyte, wherein at least some of the antigen binders are arranged in antigen binders clusters; each antigen binder is attached to one of the two or more arms that form a junction; and the binders of each antigen binder cluster are attached to the arms at uniquely addressable loci that are a predetermined distance from the junction, wherein the uniquely addressable loci are separated by predetermined intra-cluster binder distances such that the binders of each antigen binder cluster are positioned on the network of polynucleotides in a predetermined two-dimensional or three-dimensional spatial pattern that matches a two-dimensional or three-dimensional spatial pattern of the antigens in an antigen cluster.

Embodiment A8 is the artificial biopolymer complex of embodiment(s) A1, wherein: the binders from different clusters of antigen binders are separated by inter-cluster binder distances such that different antigen binder clusters are positioned on the network of polynucleotides in a predetermined two-dimensional or three-dimensional spatial pattern that matches a two-dimensional or three-dimensional spatial pattern of the antigen clusters.

Embodiment A9 is the artificial biopolymer complex of embodiment(s) A7, wherein the predetermined intra-cluster binder distances of the uniquely addressable loci of the binders of each antigen binder cluster is between 1 nm and 15 nm.

Embodiment A10 is the artificial biopolymer complex of embodiment(s) A8, wherein the predetermined inter-cluster binder distances of the antigen binder clusters is between 5 nm and 25 nm.

Embodiment A11 is an artificial biopolymer complex comprising: a network of polynucleotides comprising connector chains of polynucleotides attached to a structural chain of polynucleotides, wherein the connector chains are shorter than the structural chain; antigen binders attached to a first surface of the network of polynucleotides, wherein: the antigen binders bind to antigens of a target analyte; and the antigen binders are attached at uniquely addressable loci on the structural chain via the connector chains, wherein the uniquely addressable loci are separated by predetermined inter-binder distances such that the antigen binders are positioned on the network of polynucleotides in a predetermined two-dimensional or three-dimensional spatial pattern that matches a two-dimensional or three-dimensional spatial pattern of the antigens on the target analyte.

Embodiment A12 is the artificial biopolymer complex of embodiment(s) A11, wherein at least a portion of each chain of the connector chains is complementary to at least a portion of the structural chain, and the complementary portions of the connector chains and structural chain are hybridized to connect the connector chains to the structural chain.

Embodiment A13 is the artificial biopolymer complex of embodiment(s) A11-12, wherein a network of polynucleotides forms a tile, a tube, or a tetrahedron.

Embodiment A14 is an artificial biopolymer complex of embodiment(s) A12 wherein the two-dimensional or three-dimensional spatial pattern of the antigens is defined by intermolecular spacing of the antigens on a surface of the target analyte, and wherein the predetermined inter-binder distances of the uniquely addressable loci of the antigen binders match the intermolecular spacing of the antigens such that the antigen binders on the first surface of the network of polynucleotides align spatially with the antigens on the surface of the target analyte.

Embodiment A15 is the artificial biopolymer complex of embodiment(s) A14, wherein: each of the antigens is (i) a length and width in angstroms or nanometers from other antigens on the target analyte or (ii) a length, width, and depth from the other antigens on the target analyte, which define the intermolecular spacing of the antigens; and each antigen binder is (i) a length and width in angstroms or nanometers from other antigen binders on the network of polynucleotides or (ii) a length, width, and depth in angstroms or nanometers from the other antigen binders on the network of polynucleotides, which defines the predetermined inter-binder distances of the uniquely addressable loci of the antigen binders; and the predetermined inter-binder distances of the uniquely addressable loci of the antigen binders match the intermolecular spacing of the antigens such that the antigen binders on the network of polynucleotides align spatially with the antigens on the surface of the target analyte.

Embodiment A16 is the artificial biopolymer complex of embodiment(s) A11, wherein the antigens form antigen clusters on the target analyte, wherein at least some of the antigen binders are arranged in antigen binder clusters; each binder of an antigen binder cluster is attached to uniquely addressable loci on the structural chain via the connector chain, wherein the uniquely addressable loci are separated by predetermined intra-cluster binder distances such that each antigen binder cluster are positioned on the network of polynucleotides in a predetermined two-dimensional or three-dimensional spatial pattern that matches a two-dimensional or three-dimensional spatial pattern of the antigens in an antigen cluster on the target analyte.

Embodiment A17 is the artificial biopolymer complex of embodiment(s) A11, wherein the binders of different clusters of antigen binders are separated by a predetermined inter-cluster distance such that antigen binder clusters are positioned on the network of polynucleotides in a predetermined two-dimensional or three-dimensional spatial pattern that matches a two-dimensional or three-dimensional spatial pattern of the antigen clusters.

Embodiment A18 is the artificial biopolymer complex of embodiment(s) A16, wherein: each antigen binder in an antigen binder cluster is (i) a length and width in angstroms or nanometers from other binders of the antigen binder cluster on the network of polynucleotides or (ii) a length, width, and depth in angstroms or nanometers from the other binders of each antigen binder cluster on the network of polynucleotides, which defines the predetermined intra-cluster binder distances of the uniquely addressable loci of the binders in each antigen binder cluster; and the predetermined intra-binder distances of the uniquely addressable loci of the binders of each antigen binder cluster match intermolecular spacing of the antigens in each cluster such that the binders in the antigen binder cluster on the network of polynucleotides align spatially with the antigens in an antigen cluster.

Embodiment A19 is the artificial biopolymer complex of embodiment(s) A16, wherein the predetermined intra-cluster binder distances of the uniquely addressable loci of each antigen binder cluster is between 1 nm and 15 nm.

Embodiment A20 is a method of detecting an antigen that is indicative of a presence of a disease (for example, a cancer-specific antigen) comprising contacting one or more of the artificial biopolymer complex disclosed in this application with a target analyte, wherein the artificial biopolymer complex comprise binders that bind to the antigen, and detecting the binding of the artificial biopolymer complex to the target analyte.

Embodiment A21 is the artificial biopolymer complex of any one of embodiment(s)s 1-20, wherein the network of polynucleotides has a curvature such that the antigen binders are at least partially enveloped by a surface of the network of polynucleotides.

Embodiment A22 is the artificial biopolymer complex of any one of embodiment(s)s 1-20, wherein each of the antigens comprises one or more epitopes or domains; the first set of binders are arranged in sets of clustered antigen binders; each binder of a set of clustered antigen binders is attached to one of the two or more arms that form a junction; and the binders of each of the sets of clustered antigen binders are attached to the arms at uniquely addressable loci that are a predetermined distance from the junction, wherein the uniquely addressable loci are separated by predetermined intra-binder distances such that each set of clustered antigen binders are positioned on the network of polynucleotides in a predetermined two-dimensional or three-dimensional spatial pattern that matches a two-dimensional or three-dimensional spatial pattern of the one or more epitopes on an antigen.

Embodiment A23 is the artificial biopolymer complex of embodiment(s) A22, wherein the two-dimensional or three-dimensional spatial pattern of the one or more epitopes is defined by intramolecular spacing of the one or more epitopes on a surface of the antigen.

Embodiment A24 is the artificial biopolymer complex of embodiment(s) A23, wherein the predetermined intra-binder distances of the uniquely addressable loci of the binders of each of the sets of clustered antigen binders match the intramolecular spacing of the one or more epitopes such that the sets of clustered antigen binders on the first surface of the network of polynucleotides align spatially with the epitopes on the surface of the antigens.

Embodiment A25 is the artificial biopolymer complex of embodiment(s) A23, wherein each of the one or more epitopes is (i) a length and width in angstroms or nanometers from other epitopes of the one or more epitopes on the antigen or (ii) a length, width, and depth from the other epitopes of the one or more epitopes on the antigen, which define the intramolecular spacing of the one or more epitopes or domains; and each of the binders of each of the sets of clustered antigen binders is (i) a length and width in angstroms or nanometers from other binders of each of the sets of clustered antigen binders on the network of polynucleotides or (ii) a length, width, and depth in angstroms or nanometers from the other binders of each of the sets of clustered antigen binders on the network of polynucleotides, which defines the predetermined intra-binder distances of the uniquely addressable loci of the binders of each of the sets of clustered antigen binders; and the predetermined intra-binder distances of the uniquely addressable loci of the binders of each of the sets of clustered antigen binders match the intermolecular spacing of the epitopes such that each of the sets of clustered antigen binders on the first surface of the network of polynucleotides align spatially with the epitopes on the surface of the antigens.

Embodiment A26 is the artificial biopolymer complex of any one of embodiment(s)s 1-21, wherein the network of polynucleotides has a first surface and a second surface, wherein the first surface is attached with the antigen binders, and the second surface is attached with biomarker binders, wherein the biomarker binders bind to biomarkers on immune cells, wherein the uniquely addressable loci at which the biomarker binders are attached are separated by predetermined inter-binder distances such that biomarker binders are positioned on the network of polynucleotides in a predetermined two-dimensional or three-dimensional spatial pattern that matches a two-dimensional or three-dimensional spatial pattern of the biomarkers on the immune cells.

Embodiment A27 is the artificial biopolymer complex of any one of embodiment(s)s 1-20, further comprising one or more therapeutic agents covalently or non-covalently attached to the network of polynucleotides.

Embodiment A28 is the artificial biopolymer complex of any one of embodiment(s)s 1-20, wherein the one or more therapeutic agents include a chemotherapy agent, a small molecule drug, a siRNA, nanoparticle, and/or a protein.

Embodiment A29 is the artificial biopolymer complex of any one of embodiment(s)s 1-20, wherein the antigen binders are nucleic acids and optionally the nucleic acids are aptamers, wherein the one or more therapeutic agents comprise a DNA intercalating agent, and wherein the DNA intercalating agent is intercalated into the aptamers.

Embodiment A30 is the artificial biopolymer complex of embodiment(s) A29, wherein the antigen binders are aptamers, and wherein the DNA intercalating agent is intercalated into the aptamers.

Embodiment A31 is the artificial biopolymer complex of any one of embodiment(s)s 1-30, wherein the network of polynucleotides has been cross-linked to stabilize the structure and increase a half-life of a therapeutic agent.

Embodiment A32 is the artificial biopolymer complex of any one of embodiment(s)s 1-30, wherein the network of polynucleotides has been modified with polymers, peptides, proteins, lipids, or a combination hereof to modulate pharmacokinetics and distribution in vivo.

Embodiment A33 is the artificial biopolymer complex of any one of embodiment(s)s 1-32, wherein the antigen binders bind to the same antigen.

Embodiment A34 is the artificial biopolymer complex of any one of embodiment(s)s 1-32, wherein the antigen binders bind to two, three, or more different antigens.

Embodiment A35 is the artificial biopolymer complex of 34, wherein the first antigen and/or the second antigen are tumor-specific antigens.

Embodiment A36 is the artificial biopolymer complex of 34, wherein the ratio of the first antigen binders to the second antigen binders matches the ratio of the first antigen to the second antigen on the target analyte.

Embodiment A37 is the artificial biopolymer complex of 34, wherein the spatial arrangements of clusters of first antigen binders in relation to clusters of second antigen binders matches the spatial arrangements of first antigen clusters to second antigen clusters on the target analyte.

Embodiment A38 is a method for treating a subject, the method comprising: obtaining the artificial biopolymer complex of any one of embodiment(s)s 1-35; and administering the artificial biopolymer complex to the subject in an amount sufficient to provide a treatment effect.

Embodiment A39 is the method of embodiment(s) A38, wherein the binding of the antigen binders to the antigens to immobilize cell surface antigens in a pattern that results in a treatment effect.

Embodiment A40 is the method of embodiment(s) A38, wherein the binding of the antigen binders to the antigens activates a response by the target analyte to the artificial biopolymer complex.

Embodiment A41 is the method of embodiment(s) A40, wherein the response is a cellular internalization of the artificial biopolymer complex by the target analyte.

Embodiment A42 is the method of embodiment(s) A41, wherein the biopolymer comprises one or more therapeutic agents attached to the network of polynucleotides, and wherein the cellular internalization of the artificial biopolymer complex by the target analyte causes the one or more therapeutic agents being released from the network of polynucleotides.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process.

The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

The following examples are included for illustration but not for limitation of various embodiments of the disclosure.

### Example 1: Aptamers Binding to Cells

This example demonstrates the exemplary aptamers are capable of binding endogenously expressed targets on cells.

Human acute myeloid leukemia (AML) cell lines KASUMI-1 (CD117⁺ CD123⁺), MM1S (CD117⁻ CD123⁻), HL-60 (CD117⁻, CD123⁺), and HEL92.1.7 (CD117⁺, CD123⁻) were cultured. Five aptamers: ABWT050902 (a CD117 binder), ABWT050904 (a CD117 binder), #1 (a CD117 binder), ZW25 (a CD123 binder), and CY30 (a CD123 binder) were chemically synthesized.

Each of the aptamers was labeled with a 5′ FAM fluorophore. Aptamers were folded (heated to denature any preformed secondary and tertiary structure, then cooled and “refolded” under controlled conditions) and incubated with the cells described above. Samples were applied to a flow cytometer for sorting and gating. Histograms were produced to demonstrate the ability of the aptamers to bind to the cells.

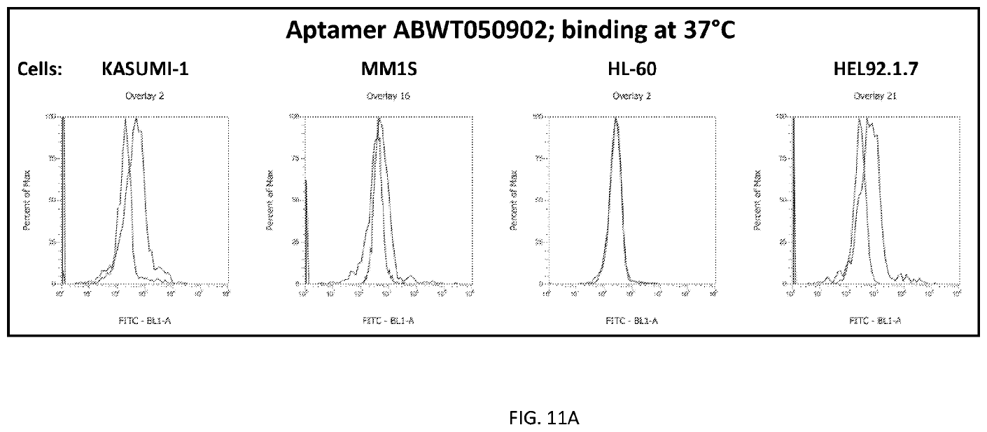

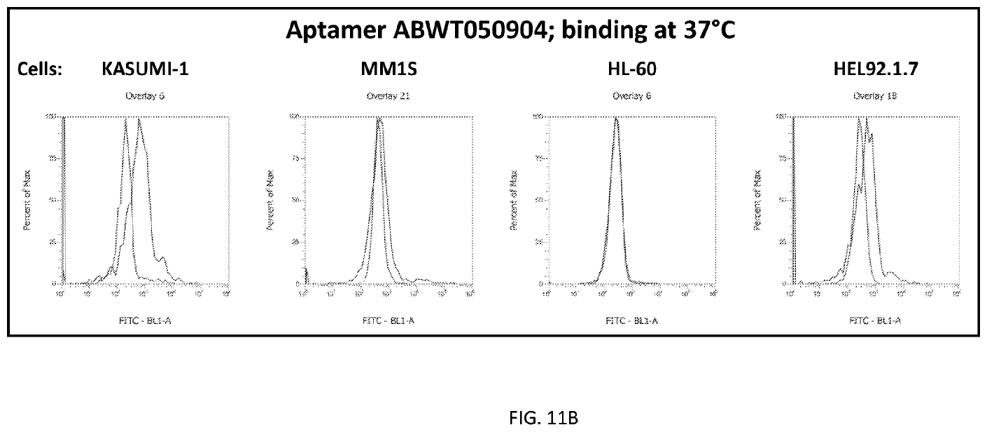

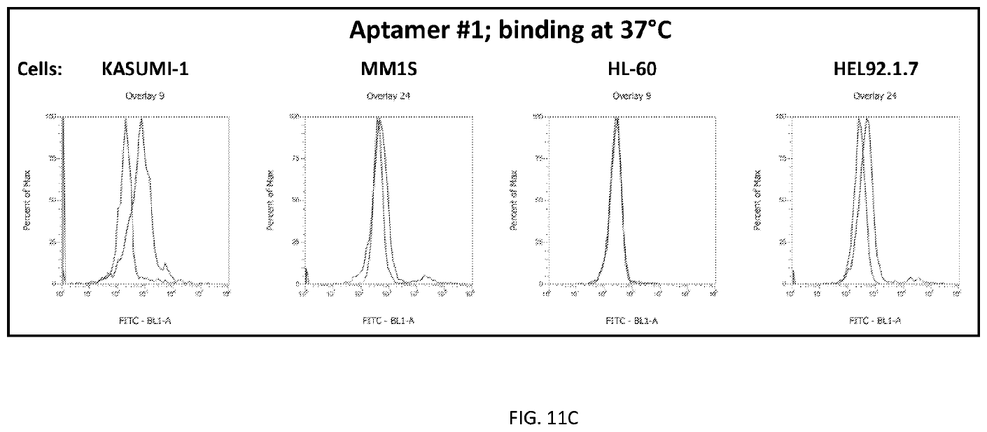

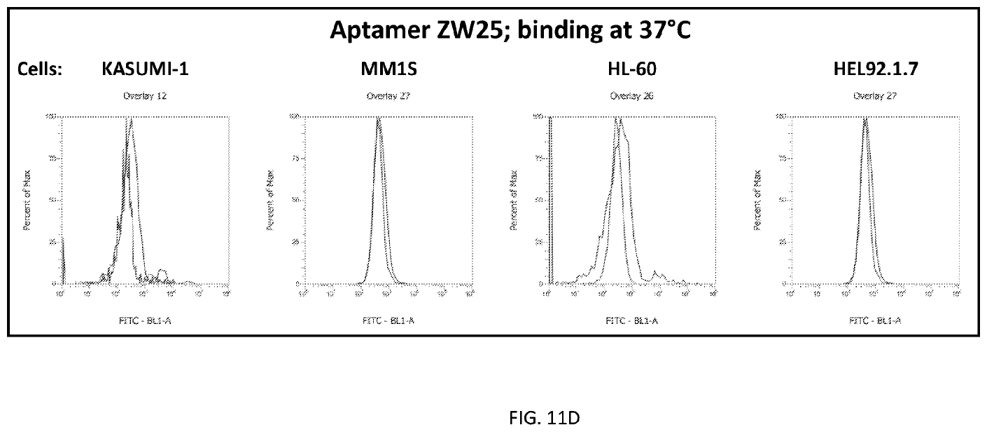

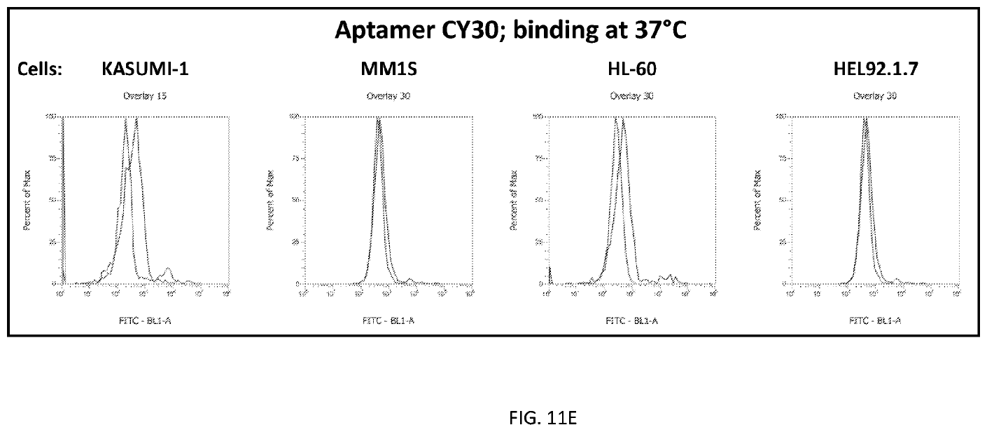

As shown in FIG. 11A-11E, a spread observed between the dark red and bright red peaks indicate that the aptamer labeled the cells (the aptamers bear the fluorophore FAM, which has similar excitation and emission spectra as FITC). The results show that ABWT050902 (a CD117 binder), ABWT050904 (a CD117 binder), #1 (a CD117 binder), ZW25 (a CD123 binder), and CY30 (a CD123 binder)—were able to bind to their respective targets that were endogenously expressed on the surfaces of AML cells. The aptamers bound to these cells in a pattern that is consistent with the cell's expression of CD117 and CD123. The results are shown in Table 1 below.

TABLE 1

| | ABWT050902 | ABWT050904 | #1 | ZW25 | CY30 |
|---|---|---|---|---|---|
| KASUMI-1 | + | + | + | − | − |
| HEL92.1.7 | + | + | + | − | − |
| HL60 | − | − | − | + | + |
| MM1S | − | − | − | − | − |

These aptamers were deemed suitable for use on networks of polynucleotides and/or selected for tumor cell killing studies as described in Examples below.

### Example 2: The Binding Affinity of the Exemplary Aptamers to Tumor Cells

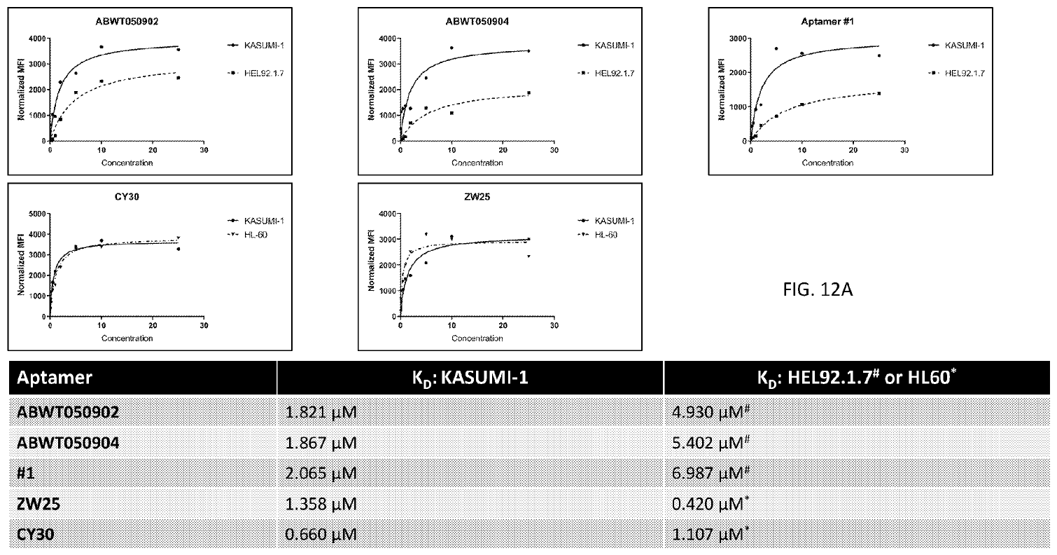

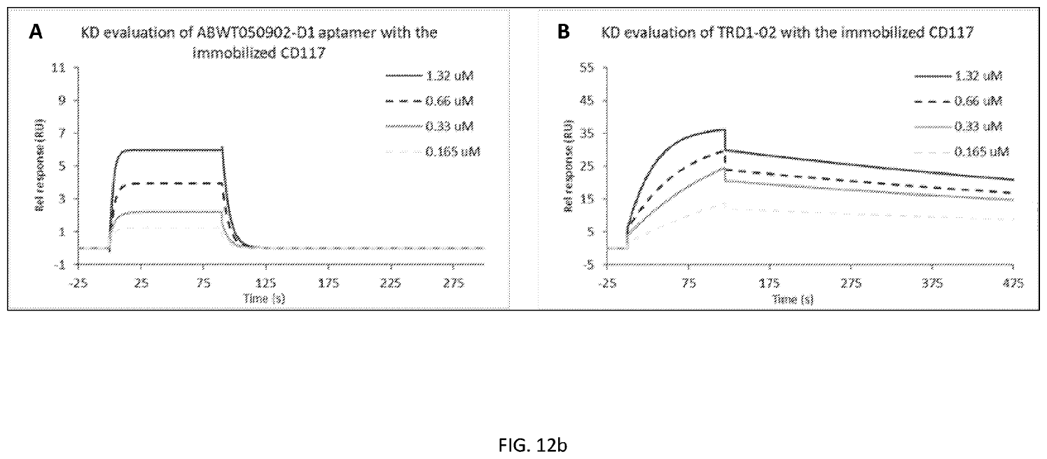

Human AML cells were cultured in preparation for binding studies with the five aptamers disclosed in Example 1. For each aptamer, cells were incubated with aptamer across 8 concentrations in triplicate. After washing with buffer, the samples were applied to a flow cytometer and data was processed into histograms as in Example 1. The mean fluorescence intensity (MFI) was determined for each sample, which were then plotted onto individual charts and subjected to hyperbolic linear regression, which determined the KDs for each aptamer. KDs were determined for each aptamer, against each cell line. As shown in FIG. 12A, the dissociation constant (K_{D}) of the interaction between the aptamers and acute myeloid leukemia cells were in the range from 10⁻⁷ M to 10⁻⁶ M. These results show that aptamers used in this study not only can bind to their targets endogenously expressed on cell surfaces but the binding was within the expected range for aptamers (μM to nM).

Next, surface plasmon resonance (SPR) assays were performed to assess the ability of networks of polynucleotides to improve the binding affinity of an aptamer. Aptamer ABWT050902 (a CD 117 binder) was folded as previously described. Aptamers alone, and aptamers on a TRD1 (as illustrated in FIG. 2) were prepared at varying concentrations and flowed over a CM5 sensor chip on a Biacore SPR immobilized with CD117. The inter-binder distances of the 96 uniquely addressable docking loci for CD 117 binders was 5 nm. Binding sensorgrams obtained and KDs determined are shown in FIG. 12B. Concentrations were normalized to total aptamer amount. Binding of ABWT050902 alone to CD 117 showed a K_{D} about 1220 nM, while binding of ABWT050902 immobilized on TRD1 to CD 117 showed a K_{D} of about 0.47 nM. These results demonstrate that for aptamer ABWT050902, immobilization on TRD1 enhances the aptamer's affinity for CD117, about 2,600-fold (from 1220 nM for aptamer alone to 0.47 nM for aptamer on TRD1). The results demonstrate that networks of polynucleotides can be used to improve individual binder affinity via avidity.

### Example 3: Daunorubicin-Loaded Aptamers Kill Target Tumor Cells

Daunorubicin-loaded aptamers were prepared loaded with daunorubicin by incubating each of the five aptamers disclosed in Example 1 with daunorubicin. Daunorubicin were allowed to diffuse and intercalate into the aptamers. Excessive molecules of daunorubicin were removed by for example, size exclusion chromatography or filtration.

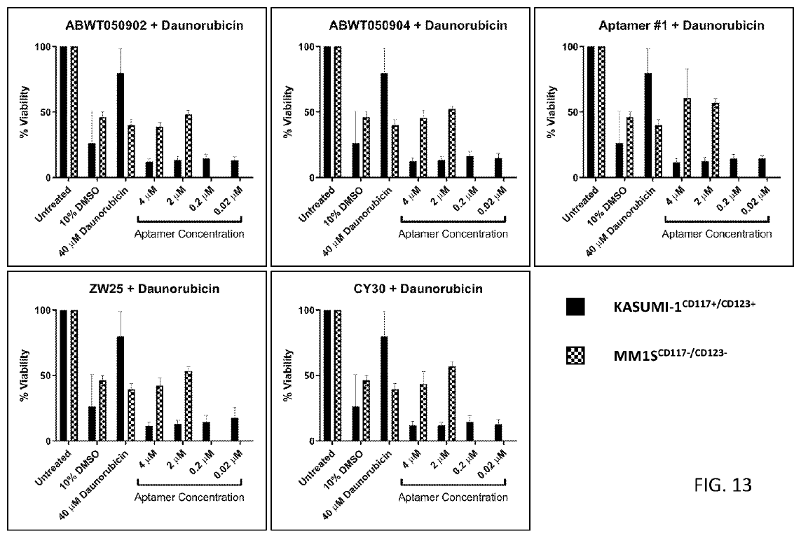

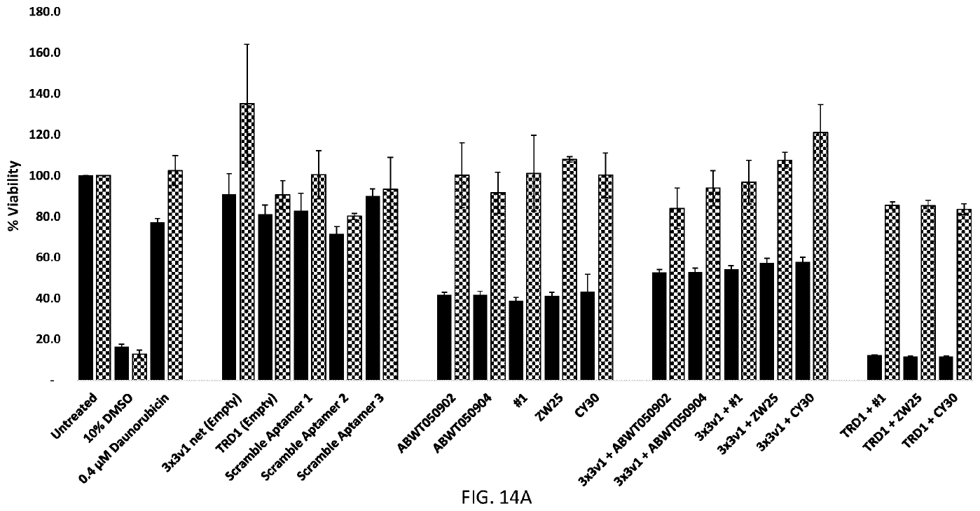

Daunorubicin-loaded aptamers were then diluted across various concentrations: 4 μM, 2 μM, 0.2 μM, and 0.02 μM. Untreated cells were used as a negative killing control. 10% DMSO was used as a positive killing control. 40 μM daunorubicin was also used as a positive killing control but one that matched the current National Comprehensive Cancer Network (NCCN) guidelines for AML daunorubicin chemotherapy dosing. Cells were incubated with their respective treatments for 24 hours before they were washed with cell medium. After 48 hours, the viability of all cells were assessed using MTT assays. All conditions were performed in triplicate for standard error analysis. MM1S cells were used as negative control in the assays having aptamers at 4 μM and 2 μM, respectively. The results are shown in FIG. 13.

As shown in FIG. 13.1, daunorubicin produced approximately 50% killing of negative control cell line MM1S (no expression of CD117 or CD123). The incubation of daunorubicin-loaded aptamers with MM1 S did not enhance this killing effect. This is consistent with the fact that these aptamers were unable to bind to MM1S cells and thus did not result in enhanced killing. In contrast, daunorubicin produced approximately 20% killing of KASUMI-1 cells. KASUMI-1 cells express CD 117 and CD123. The killing effect for KASUMI-1 was potentiated by aptamer binding, as indicated by an increase of killing from approximate 20% to approximate 90%. This is consistent with the fact these daunorubicin-loaded aptamers are able to bind to CD117 and CD123 expressed on the KASUMI-1 cells, a type of AML cells. This experiment demonstrated the ability of the aptamers chosen for this study to specifically kill AML cells, as well as their appropriateness for their immobilization on networks of polynucleotides.

### Example 4: Target Cell Killing by Daunorubicin-Loaded Aptamers Docked on a Network of Polynucleotides

This example demonstrates a network of polynucleotides disclosed herein can enhance the ability of aptamers to deliver daunorubicin to AML cells and kill AML cells.

The networks of polynucleotides used in Examples of this application include DNA origami (TRD1 and PTR) and DNA nets (3x3v1, 3x3v2). See Table 2

TABLE 2
Networks of polynucleotides

| | Network | Network | Network | Number of | Structural | Connector |
|---|---|---|---|---|---|---|
| Network | Type | Length | Width | Binder Loci | Chain Length | Chain Length |
| TRD1 | Origami | 90 nm | 75 nm | 96 | 7249 nts | 32 nts (no |
| | | | | | | dock site); |
| | | | | | | 47 nts (with |
| | | | | | | dock site) |
| PTR45 | Origami | 90 nm | 75 nm | 76 | 7249 nts | 32 nts (no |
| | | | | | | dock site); |
| | | | | | | 47 nts (with |
| | | | | | | dock site) |
| PTR86 | Origami | 90 nm | 75 nm | 40 | 7249 nts | 32 nts (no |
| | | | | | | dock site); |
| | | | | | | 47 nts (with |
| | | | | | | dock site) |
| 3x3v1 | Net | 60 nm | 60 nm | 27 | 43 to 46 nts | N/A |
| 3x3v2 | Net | 60 nm | 60 nm | 9 | 43 to 46 nts | N/A |

note:
“nt” is short for “nucleotide”;
“Network” is short for “Network of polynucleotides”

Daunorubicin-loaded aptamers were prepared as described in Example 3. The killing activity of daunorubicin-loaded aptamers alone and daunorubicin-loaded aptamers on various networks of polynucleotides, i.e., 3x3v1 nets and TRD1 tiles on KASUMI-1 cells (an AML cell line) were assessed using the procedures as described in Example 3. Additional negative killing controls were added to control for any inherent nonspecific killing effects by the networks of polynucleotides or aptamers: “3x3v1 net (Empty)” is 3x3v1 net without aptamers (therefore, no daunorubicin), “TRD1 (Empty)” is TRD1 without aptamers (therefore, no daunorubicin), “Scramble Aptamer 1”, “Scramble Aptamer 2”, and “Scramble Aptamer 3” were three oligos with lengths and A:T:C:G ratios similar to the tested aptamers but whose nucleotide sequences were randomized but also loaded with daunorubicin. 0.4 μM Daunorubicin were intercalated into aptamers across all conditions.

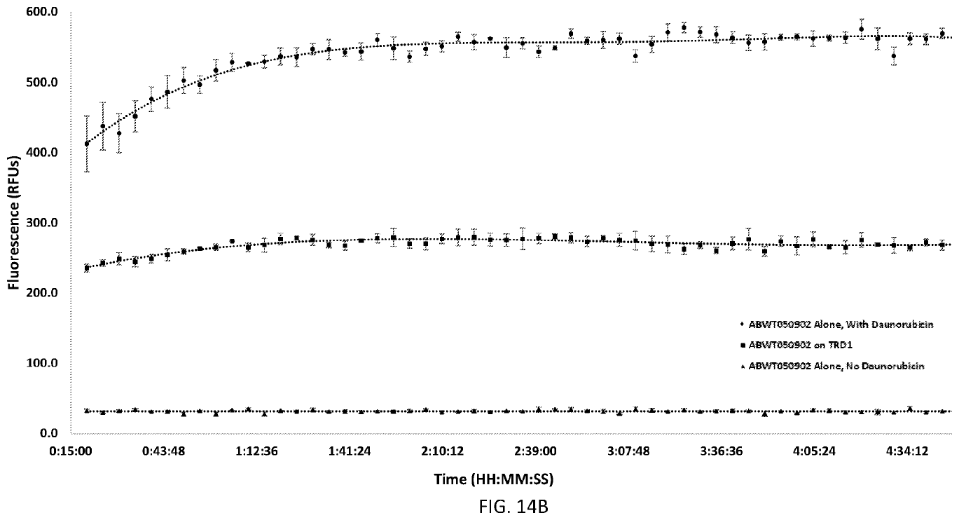

As shown in FIG. 14, across all the conditions except for the positive cell killing condition “10% DMSO”, almost no non-specific killing was observed for MM1S cells. For KASUMI-1 cells, almost no killing was observed from the empty network of polynucleotides and scrambled aptamer negative controls (at most, approximately 20%). Specific cell killing was again seen for the aptamers alone after 3 hours incubation (unlike 24 hours in the previous experiment): approximately 50% killing of KASUMI-1 cells. During the 3-hour incubation, 3x3v1 nets did not appear to potentiate this effect. However, TRD1 potentiated the killing effect to approximately 90% cell killing. The results indicate that TRD1 potentiated the daunorubicin-loaded aptamer killing of targeted KASUMI-1 cells while inducing low nonspecific toxicity (as indicated by maintaining MM1S toxicity to below 20%). This experiment demonstrates that the geometries of the network of polynucleotides disclosed in the application are able to enhance not only the avidity of aptamers as shown in previous examples but also their cell-killing abilities.

### Example 5: Target Cell Killing by Daunorubicin-Loaded Aptamers Displayed on a Network of Polynucleotides

The experiments in this example were performed under similar assay conditions to what were used in Example 4, except that the K_{D} values were calculated to quantitatively compare the cell killing effects of daunorubicin-loaded aptamers alone, on 3x3v1 net, and TRD1.

KASUMI-1 cells were cultured and incubated with all five aptamers (each loaded with daunorubicin) across six concentrations: 10 nM, 1 nM, 100 μM, 10 μM, 1 μM and 100 fM. 10% DMSO, 40 μM daunorubicin, 4 μM daunorubicin, and 0.4 μM daunorubicin were used as positive killing controls. 3x3v1 net (Empty), TRD1 (Empty), Scramble Aptamer 1, Scramble Aptamer 2, and Scramble Aptamer 3 were negative killing controls. Incubation and washing procedures were as described in Experiment 3. After 48 hours, the samples were measured for viability via MTT assay. Data points were plotted and unpaired student's t—tests were performed to determine significance. For IC₅₀ calculations, the concentrations were transformed to the logarithmic equivalent followed by normalization of the relative cell viability on a scale of 0-100. The normalized viability was subjected to nonlinear regression against the logarithmically converted inhibitor concentrations in the three parameters variable slope mode to determine the IC₅₀. IC₅₀s are shown in Table 3 and Table 4.

TABLE 3
IC₅₀s of daunorubicin and aptamers alone and immobilized
on various networks of polynucleotides

| | | | ΔFold over | | ΔFold over |
|---|---|---|---|---|---|
| IC₅₀ (pM) | Free | 3x3v1 | Free | TRD1 | Free |
| Daunorubicin | 58,400 | — | — | — | — |
| ABWT050902 | 529 | 218 | 2.4x | — | — |
| ABWT050904 | 976 | 233 | 4.2x | — | — |
| #1 | 531 | 147 | 3.6x | 95 | 5.6x |
| ZW25 | 3,542 | 445 | 8.0x | 102 | 34.7x |
| CY30 | 5,073 | 931 | 5.5x | 133 | 38.1x |

TABLE 4
IC₉₀s of aptamers alone and immobilized
on networks of polynucleotides

| IC₉₀ (pM) | Free | 3x3v1 | TRD1 |
|---|---|---|---|
| ABWT050902 | N/A | 61,800 | — |
| ABWT050904 | N/A | 17,600 | — |
| #1 | 108,000 | 208,000 | 165,000 |
| ZW25 | N/A | 128,000 | 7830 |
| CY30 | N/A | 8,700 | 93,600 |

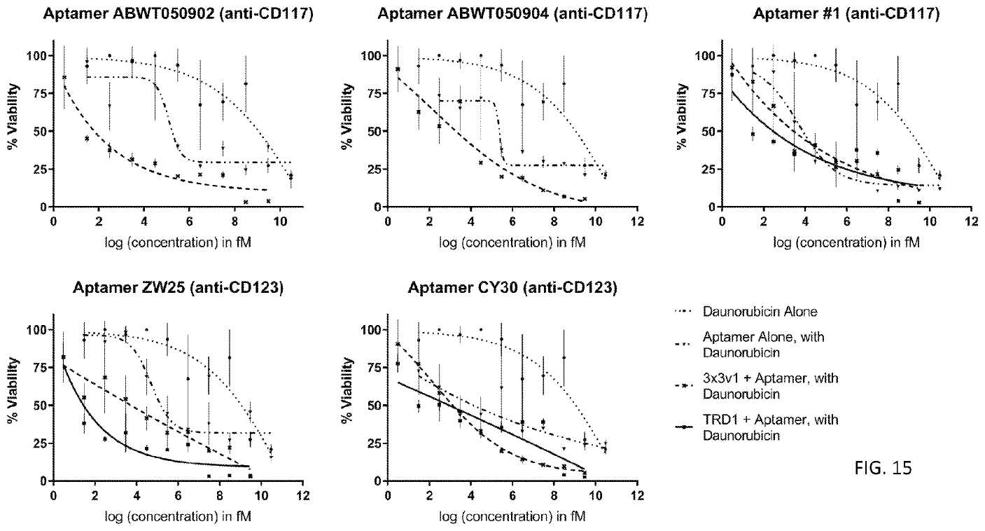

As shown in FIG. 15, daunorubicin-loaded aptamers displayed on various networks of polynucleotides potentiate their cell-killing effects against AML cells, as evidenced by reduced the IC₅₀ values of aptamers on networks of polynucleotides compared to aptamers alone. The data also demonstrates the effects of geometries of networks of polynucleotides on cell killing effect. Of interest is the differential performance of the 3x3v1 and TRD1. The aptamer spacing on both 3x3v1 nets and TRD1 is 5 nm. However, aptamers on 3x3v1 nets are arranged in clusters with an inter-cluster binder distance of 15 nm. Comparatively, aptamers on TRD1 are not arranged in clusters in this example and are evenly spaced across the TRD1 surface. Though 3x3v1 net provided an IC₅₀ enhancement over aptamer alone, it is marginal, i.e., below a 10-fold improvement. This is exemplified by CD123-binding aptamers ZW25 and CY30. The widest dimension on CD123 is ˜5 nm as measured on PDB 4JZJ; (doi.org/10.2210/pdb4JZJ/pdb; Broughton, S. E., et al., (2014) Cell Rep 8: 410-419) using the distance measurement tool in UCSF Chimera 1.16. When arranged on 3x3v1 nets, ZW25 and CY30 only improve IC₅₀ 8.0-fold and 5.5-fold, respectively, compared to free ZW25 and free CY30, respectively. However, when these aptamers were immobilized on TRD1, IC₅₀ improves by 34.7-fold and 38.1-fold, respectively. This demonstrates the importance of binder and cluster spacing, as well as the geometry of the network of polynucleotides in the design of DNA nanostructures optimized for a biological effect.

Also of interest are the curve forms of several of the aptamers, specifically ABWT050902, ABWT050904, #1, and ZW25. When these aptamers are free in solution, their inhibition curves are sigmoidal, demonstrating that with increasing concentration, free aptamer biological activity decreases, likely due to increasing inter-aptamer interactions that can disrupt aptamer conformations and/or inhibit target binding. When immobilized on a network of polynucleotides (e.g., 3x3v1 or TRD1), these sigmoidal curves flatten into hyperbolic curves, demonstrating a more even trend between aptamer concentration and biological activity. This is likely due to the advantages that immobilization of the network of polynucleotides provides to aptamer binding activity. When immobilized, aptamer spacing is controlled, which mitigates disruptive inter-aptamer concentrations, allowing for a more direct relationship between aptamer concentration and biological activity.

### Example 6: Formation of Protein G-Docking Oligo Conjugates

This example demonstrates the functionalization of a network of polynucleotides with a non-aptamer binder, i.e., a protein.

We used a sulfo-SMCC linker to conjugate an oligo (complementary to 3x3v1 docking sites) bearing a 5′ amine to the N terminal cysteine sulfhydryl R group on 6xHis-tagged recombinant Protein G (the oligo also bore a 3′ biotin for later purification). See FIG. 16A. This was performed at room temperature in 1x phosphate buffered saline (PBS), pH 7.4. Oligo functionalization with sulfo-SMCC was performed first (as is typical), followed by two Zeba column (7 kDa molecular weight cutoff) cleanups to remove the excess sulfo-SMCC. Functionalized oligos were then placed in molar excess to Cys-Protein G. Purification proceeded via usage of monomeric avidin resin (to remove any unconjugated Cys-Protein G) followed by Ni²⁺-NTA resin (to remove any unconjugated oligos). Oligo-Protein G conjugates were then docked to 3x3v1 (27 docking sites) and 3x3v2 (9 docking sites) nets and assayed for activity on SPR binding assays.

3x3v1 net was used in the experiment shown in FIG. 16B. This net has 27 docking sites. 3x3v2 net was used in the experiment shown in FIG. 16C. This net has 9 docking sites. Despite 3x3v1 net having 27 available docking sites, the agarose gel image in FIG. 16B demonstrates a near-saturation of docking sites when the molar ratio of 3x3v1 net to Oligo-Protein G Conjugate is 1:16 (i.e., 27 docking sites:16 conjugates). It is likely that this saturation occurred because of the steric hindrances involved. The docking oligo alone has a molecular mass of approximately 10,000 Daltons. Protein G has an approximate molecular mass of 27,000 Daltons. Combined, the Conjugate has an approximate molecular mass of 37,000 Daltons. Therefore, those 3x3v1 nets are able to dock 27 docking oligos per Net, when those oligos shift from approximately 10,000 Daltons to approximately 37,000 Daltons upon conjugation to Protein G, it becomes highly likely that the molecular volume of the Conjugate is not capable of fully occupying the 3x3v1 docking sites, which are on a 5 nm spacing. Because of this, we performed the docking experiment shown on the agarose gel image in FIG. 16D, which docked Conjugates to 3x3v2 net, which have 9 docking sites (instead of 3 docking sites at each Holliday junction on the 3x3 net, there is one docking site). As a result, docking Conjugates at a 9:1 molar ratio to 3x3v2 nets (9 conjugates: 9 docking sites) produced an obvious up-gel band shift compared to the 4.5:1 molar ratio (4.5 Conjugates to 9 docking sites). Moreover, an 18:1 molar ratio (18 Conjugates to 9 docking sites) did not produce additional up-gel shifting compared to the 9:1 molar ratio docking condition, demonstrating that the 9:1 ratio saturates all of the available docking sites on 3x3v2 nets. This experiment further demonstrates the optimization of the spacing of docking sites on networks of polynucleotides in consideration of dimension of the binders is helpful to maximize the occupancies of the binders on the network of polynucleotides.

### Example 7: Multi-Targeting of the Networks of Polynucleotides

This example demonstrates (i) networks of polynucleotides can be designed with the ability to dock multiple aptamers, enabling singular network of polynucleotides to have specific affinity to multiple targets and (ii) a single network of polynucleotides can be designed to target more than one target simultaneously, and through this targeting, experience higher avidity to the target cells than monotargeted networks of polynucleotides.

PTR45 (FIG. 17C) was assembled and docked with the CD117-binding aptamer ABWT050902 (bearing a D1 dock site consensus sequence) and anti-SARS-CoV-2 nucleocapsid (N) protein aptamer A15 (bearing a D2 dock site consensus sequence), referred to as the 45-PTR-02-A15 net. These aptamers and networks of polynucleotides were prepared and applied to SPR binding assays as described in Experiment 2. The key difference between these networks of polynucleotides and the TRD1 used in Experiment 2b is that the TRD1 of Experiment 2b only bore ABWT050902, whereas the PTR45 used in this experiment bear two clusters of aptamers: ABWT050902 on one side of the network of polynucleotides, and A15 on the other side.

These results demonstrate that a singular 45-PTR-02-A15—can bind to multiple targets, i.e., CD117 and SARS-CoV-2 N protein. This shows that singular networks of polynucleotides can be designed to bind multiple targets, either individually or simultaneously, endogenously expressed on cell surfaces. This also demonstrates that networks of polynucleotides can be designed such that their targeting of cancer cells can be more selective than other prior arts, such as bispecific antibodies or CAR-T cells. More specifically, networks of polynucleotides can be designed with binder clusters that encourage high affinity binding of the network of polynucleotides to cells expressing antigen profiles specific to cancer cells, while making binding to immune cells—which are likely to express one or more of the target antigens, and at varying expression levels—less likely. For example, a network of polynucleotides designed with three clusters of binders where one cluster binds CD33, another binds CD 117, and the third binds CD123, will have a lower affinity for immune cells expressing low amounts of any one of the named antigens. However, this network of polynucleotides will have high affinity for AML cells expressing all three the named antigens, enhancing the “on target, on tumor” selectivity of the network of polynucleotides.

We used the TRD1 tile structure and altered the identity and spacing of the dock sites. In FIG. 17A, every labeled square (e.g., p3_200, p1_005, p1_006, etc.) is a staple strand that has a dock site appended to it in the original TRD1 design. In FIG. 17B, the staples represented by the light green squares maintain their D1 docking sites, staples represented by the dark orange squares have been changed to D2 docking sites, and staples represented by pale yellow squares have had their dock sites removed, i.e., they are only staples in this design. Because the tile in this design is “polarized” (i.e., binders with D1 dock sites can only dock to sites on the left of the tile, and binders with D2 dock sites can only dock to sites on the right of the tile), it is called a “polarized tall rectangle”, or “PTR”. In this design, out of a total of 88 possible docking sites, only 76 sites are used, or 86% of total. Therefore, this design is called “PTR-86”. Similarly, in FIG. 17C, the number of staples bearing docking sites has been halved compared to PTR-86; 40 out of a possible 88 sites are used, or 45% of total. Therefore, this design is called “PTR-45”. These experiments were performed using conditions similar to those described in Experiment 3.

FIG. 17D shows that PTR86 could kill various AML cell lines as a function of biomarker expression and extent of targeting. Monotargeted PTR86 02:02 (only targeted to CD 117 via aptamer ABWT050902) demonstrated almost no killing of HL-60 cells (no CD117 expression), and significant (<30%) killing of HEL92.1.7 and KASUMI-1 cells (high CD 117 expression). Similarly, Monotargeted PTR86 30:30 (only targeted to CD123 via aptamer CY30) demonstrated slight killing of HEL92.1.7 cells (low CD123 expression), and significant (<30%) killing of HL-60 and KASUMI-1 cells (high CD123 expression). Dual-targeted PTR86 02D1:30D2 (targeted to CD117 via aptamer ABWT050902, and to CD123 via aptamer CY30), demonstrated significant cell killing of all three cell lines. Notably, dual-targeted PTR86 02D1:30D2 demonstrated killing of KASUMI-1 cells that was superior to the monotargeted networks of polynucleotides. Moreover, dual-targeted PTR86 02D1:30D2 demonstrated killing of KASUMI-1 cells that was superior to PTR86 02D1:30D2 killing of HL60 and HEL92.1.7 cells.

FIG. 17D demonstrates that dual-targeted PTR86 02D1:30D2 (targeting both CD 117 and CD123) caused more killing of KASUMI-1 cells than monotargeted networks of polynucleotides. FIG. 17E demonstrates that monotargeted networks of polynucleotides kill more KASUMI-1 cells than HL-60 or HEL92.1.7 due to antigen expression levels and other cellular sensitivities to daunorubicin and networks of polynucleotides across these cell lines. In addition, dual-targeted PTR02D1:30D2 kills similar percentages of HL-60 and HEL92.1.7 cells compared to monotargeted PTR86. However, dual-targeted PTR02D1:30D2 kills more KASUMI-1 cells than either of the PTR86 monotargeted to either CD 117 or CD123. This demonstrates that dual-targeted networks of polynucleotides have not only enhanced affinity and killing of cells expressing both targeted antigens compared to cells expressing only one of the targeted antigens, but also that dual-targeted networks of polynucleotides have a targeting property that can be exploited. These multitargeted networks of polynucleotides can be designed such that they can selectively discriminate between cells of varying antigen expression levels while also potentiating the cell killing effects of therapeutics.

FIG. 17E shows the differences in abilities of monotargeted and dual-targeted networks of polynucleotides to kill various AML cell lines. Comparing high CD117-expressing cell lines HEL92.1.7 and KASUMI-1, monotargeted PTR86 02:02 (targeted only to CD117; 76 ABWT050902 aptamers) demonstrates an increase of KASUMI-1 cell killing of 23.3% over HEL92.1.7; an effect caused in part by differing CD 117 expression levels and cellular sensitivities to daunorubicin and networks of polynucleotides. Comparing high CD123-expressing cell lines HL-60 and KASUMI-1, monotargeted PTR86 30:30 (targeted only to CD123; 76 CY30 aptamers) demonstrates an increase of KASUMI-1 cell killing of 22.8% over HEL92.1.7; an effect caused in part by differing CD123 expression levels and cellular sensitivities to daunorubicin and networks of polynucleotides. Dual-targeted PTR86 02D1:30D2 (targeted to both CD117 and CD123 via 38 ABWT050902 aptamers and 38 CY30 aptamers, respectively), demonstrated and increase in KASUMI-1 cell killing of 40.0% over HL-60 cells, and 35.0% over HEL92.1.7 cells.

### Example 8: The Effect of Binder Spacing on the Network of Polynucleotides

This example demonstrates the inter-binder distance on the surfaces of the network of polynucleotides affects the biological efficacy of the network of polynucleotides.

ABWT050902 aptamers with D1 dock sites were loaded with daunorubicin as described previously. Aptamers were then immobilized on PTR45 and PTR86 (please refer to FIG. 17B and FIG. 17C). PTR45 was loaded with 20 ABWT050902 D1 aptamers, whereas PTR86 were loaded with 40. KASUMI-1 cells were grown, incubated with analytes, and measured via MTT cell killing assays as described previously.

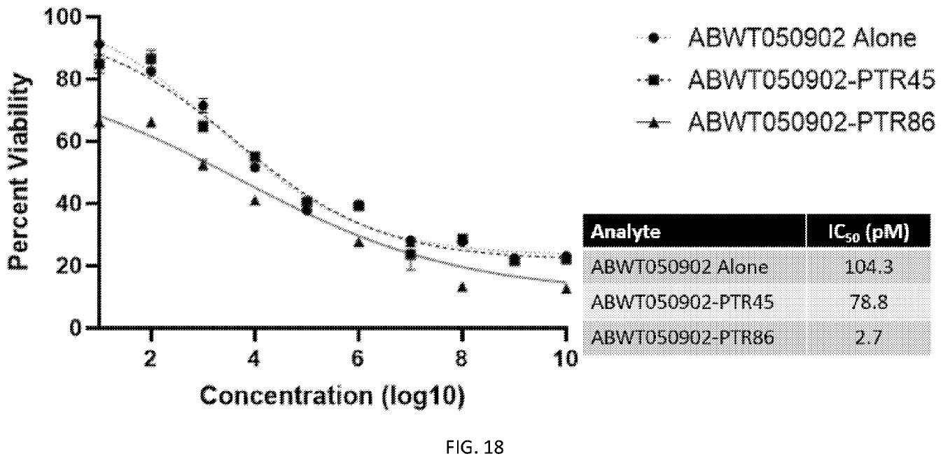

As shown in FIG. 18, the effects of ABWT050902 (alone and immobilized on the PTR including PTR86 and PTR45) on KASUMI-1 cell viability. In these results, ABWT050902 aptamers alone and ABWT050902 aptamers on PTR45 had similar biological efficacies, killing KASUMI-1 cells with similar IC₅₀s (104.3 μM compared to 78.8 μM, respectively). For ABWT050902-PTR86, IC₅₀ changed from 78.8 μM for ABWT050902-PTR45 to 2.7 μM, yielding a 39.4-fold improvement in efficacy (compared to 1.32-fold for ABWT050902-PTR45 over ABWT050902 aptamer alone).

These results demonstrate the importance and interconnectedness of avidity and binder spacing on networks of polynucleotides. It is known that at a 5 nm spacing (the spacing used on PTR86), that ABWT050902 retains binding activity to CD117. It is also known that the widest dimension on CD117 is ˜5 nm. Logically, a CD117-binding network of polynucleotides designed such that immobilized aptamers are maintained at a spacing of 5 nm—a spacing that is compatible with CD117 structural dimensions—should have superior avidity to CD117-expressing cells compared to networks of polynucleotides with a wider binder spacing. In this experiment, ABWT050902 aptamers immobilized on PTR45 with a spacing of 10 nm demonstrated marginal IC₅₀ improvement compared to ABWT050902 aptamers alone. This demonstrated that the design of ABWT050902-PTR45 was inferior to ABWT050902-PTR86. This experiment demonstrates that inferiority is only avoided upon using the non-obvious methods of the present invention, which designs networks of polynucleotides with binder arrangements that consider both binder sterics and target dimensions—a key differentiator between the present invention and the prior art.

### Example 9. Docking of Fc Domain-Bearing Binders to Networks of Polynucleotides

This example demonstrates docking binders bearing an Fc domain on a network of polynucleotides.

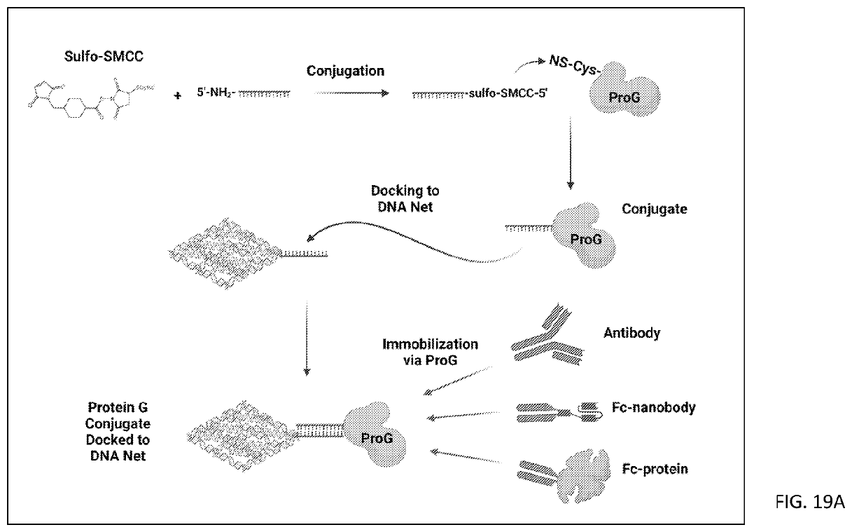

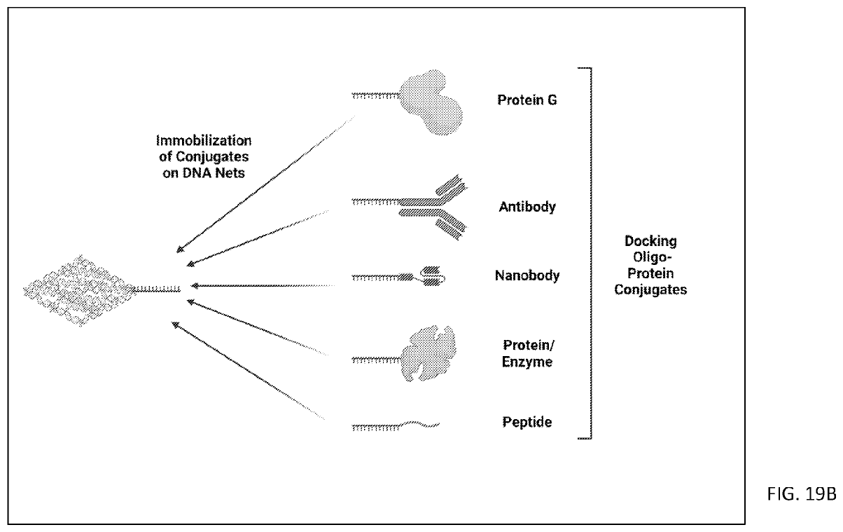

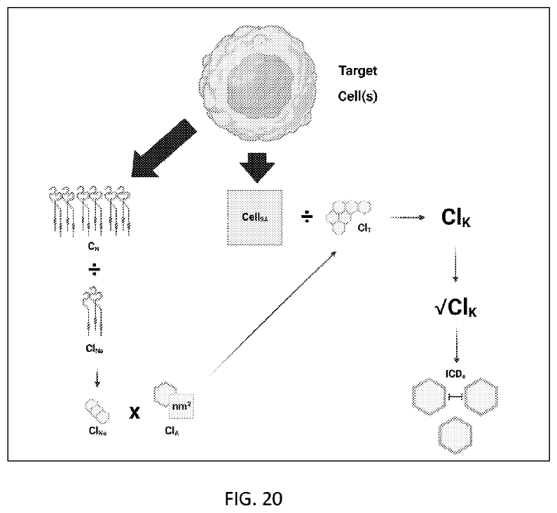

Though in most cases networks of polynucleotides rely on Watson-Crick base-pairing to immobilize—or dock—binders to the surfaces of a network of polynucleotides, it is possible to immobilize other molecular entities on networks of polynucleotides, such as antibodies and Fc-fusion proteins. Having obtained the results from Experiment 6, it will be possible to exploit the presence of Protein G on networks of polynucleotides to in turn immobilize Fc-fusion proteins and antibodies on networks of polynucleotides through Protein G's activity, i.e., its ability to non-covalently bind Fc domains. This ability is illustrated in FIG. 19A. The immobilization of antibodies and Fc-fusion proteins, such as—but not limited to—Fc-nanobodies, greatly extends the repertoire of binder classes compatible with networks of polynucleotides.

For those binders that are larger than aptamers, such as antibodies (aptamers are typically 10 kDa to 30 kDa, whereas antibodies and Fc-fusion proteins can be 150 kDa or more), the design of the hosting networks of polynucleotides and, specifically, the docking site spacing on the networks of polynucleotides requires precision beyond that required for aptamer binders. For example, an antibody at approximately 5× the size of an aptamer needs spacing in excess of 5 nm on the surfaces of a network of polynucleotides(aptamers tolerate this minimum spacing due to their compact sizes). However, antibodies bear two variable domains. Therefore, though some antibodies require approximately 10 nm spacings, the stoichiometry of 2 variable domains per antibody largely preserves the number of binder active domains on the surface of the network of polynucleotides, which maintains the potential for avidity enhancements. This example illustrates the need to consider the dimensions of the binder in the creation of polynucleotide network designs that do not sterically hinder the binder's ability to bind its target(s).

Other non-antibody binders that can be of sizes similar to aptamers can be immobilized on the surface of the network of polynucleotides. These non-antibody binders may include, but are not limited to peptides, nanobodies, or other proteins and enzymes.

In addition to the scheme described above and illustrated in FIG. 19A, the successful use of sulfo-SMCC to conjugate Protein G with a docking oligo demonstrates other proteins (for example, peptides, nanobodies, and other proteins and enzymes) can also be similarly conjugated. Protein G is not required for the docking of other proteins to networks of polynucleotides (FIG. 19B).

1. An artificial biopolymer complex comprising:
a network of polynucleotides comprising structural units connected to one another via a series of arms and junctions, wherein intersections of two or more arms form the junctions, wherein:
each of the structural units has a predetermined shape defined by one or more strands of polynucleotides;
at least a portion of the one or more strands of polynucleotides of each structural unit is complementary to at least a portion of the one or more strands of polynucleotides of another structural unit, and the complementary portions of the strands of the polynucleotides of adjacent structural units are hybridized to connect the adjacent structural units;
the complementary portions of the strands of the polynucleotides of adjacent structural units form the arms with a predetermined length; and
the intersections of the two or more arms form the junctions at a predetermined distance from one another based on the predetermined length of the arms, wherein the predetermined distance is in a range of 5 nm to 999 nm;
antigen binders attached to a surface of the network of polynucleotides, wherein:
the antigen binders bind to antigens of a target analyte; and
wherein at least some of the antigen binders are attached at uniquely addressable loci on the arms forming the junctions, wherein the uniquely addressable loci are separated by predetermined inter-binder distances such that the antigen binders are positioned on the network of polynucleotides in a predetermined two-dimensional or three-dimensional spatial pattern that matches a two-dimensional or three-dimensional spatial pattern of the antigens on the target analyte.

2. (canceled)

3. The artificial biopolymer complex of claim 1, wherein each structural unit is defined by six or more polynucleotides.

4. The artificial biopolymer complex of claim 1, wherein the two-dimensional or three-dimensional spatial pattern of the binders is defined by intermolecular spacing of the antigens on a surface of the target analyte, and wherein the predetermined inter-binder distances of the uniquely addressable loci of the antigen binders match the intermolecular spacing of the antigens such that the antigen binders on the network of polynucleotides align spatially with the antigens on the surface of the target analyte.

5. The artificial biopolymer complex of claim 4, wherein the predetermined inter-binder distances of the uniquely addressable loci of the antigen binders match the intermolecular spacing of the antigens such that the antigen binders on the surface of the network of polynucleotides align spatially with the antigens on the surface of the target analyte.

6. The artificial biopolymer complex of claim 1, wherein:
each of the antigens is (i) a length and width in angstroms or nanometers from other antigens on the target analyte or (ii) a length, width, and depth from the other antigens on the target analyte, which define intermolecular spacing of the antigens; and
each antigen binder is (i) a length and width in angstroms or nanometers from other antigen binders on the network of polynucleotides or (ii) a length, width, and depth in angstroms or nanometers from other antigen binders on the network of polynucleotides, which defines the predetermined inter-binder distances of the uniquely addressable loci of antigen binders; and
the predetermined inter-binder distances of the uniquely addressable loci of the antigen binders match the intermolecular spacing of the antigens such that the antigen binders on the surface of the network of polynucleotides align spatially with the antigens on the surface of the target analyte.

7. The artificial biopolymer complex of claim 1, wherein:
the antigens form antigen clusters on the target analyte, wherein at least some of the antigen binders are arranged in antigen binder clusters;
each antigen binder is attached to one of the two or more arms that form a junction; and
the binders of each antigen binder cluster are attached to the arms at uniquely addressable loci that are a predetermined distance from the junction, wherein the uniquely addressable loci are separated by predetermined intra-cluster binder distances such that the binders of each antigen binder cluster are positioned on the network of polynucleotides in a predetermined two-dimensional or three-dimensional spatial pattern that matches a two-dimensional or three-dimensional spatial pattern of the antigens in an antigen cluster.

8. The artificial biopolymer complex of claim 7, wherein:
wherein the binders from different clusters of antigen binders are separated by inter-cluster binder distance such that different antigen binder clusters are positioned on the network of polynucleotides in a predetermined two-dimensional or three-dimensional spatial pattern that matches a two-dimensional or three-dimensional spatial pattern of the antigen clusters.

9. The artificial biopolymer complex of claim 7, wherein each of the predetermined intra-cluster binder distances of the uniquely addressable loci of the binders of each antigen binder cluster is between 1 nm and 15 nm.

10. (canceled)

11. An artificial biopolymer complex comprising:
a network of polynucleotides comprising connector chains of polynucleotides attached to a structural chain of polynucleotides, wherein the connector chains are shorter than the structural chain, wherein at least a portion of each chain of the connector chains is complementary to at least a portion of the structural chain, and the complementary portions of the connector chains and structural chain are hybridized to connect the connector chains to the structural chain;
antigen binders attached to a first surface of the network of polynucleotides, wherein:
the antigen binders bind to antigens of a target analyte; and
the antigen binders are attached at uniquely addressable loci on the structural chain via the connector chains, wherein the uniquely addressable loci are separated by predetermined inter-binder distances such that the antigen binders are positioned on the network of polynucleotides in a predetermined two-dimensional or three-dimensional spatial pattern that matches a two-dimensional or three-dimensional spatial pattern of the antigens on the target analyte.

12. (canceled)

13. The artificial biopolymer complex of claim 11, wherein the network of polynucleotides forms a tile, a tube, or a tetrahedron.

14. The artificial biopolymer complex of claim 11, wherein the two-dimensional or three-dimensional spatial pattern of the antigens is defined by intermolecular spacing of the antigens on a surface of the target analyte, and wherein the predetermined inter-binder distances of the uniquely addressable loci of the antigen binders match the intermolecular spacing of the antigens such that the antigen binders on the first surface of the network of polynucleotides align spatially with the antigens on the surface of the target analyte.

15. The artificial biopolymer complex of claim 14, wherein:
each of the antigens is (i) a length and width in angstroms or nanometers from other antigens on the target analyte or (ii) a length, width, and depth from the other antigens on the target analyte, which define the intermolecular spacing of the antigens; and
each antigen binder is (i) a length and width in angstroms or nanometers from other antigen binders on the network of polynucleotides or (ii) a length, width, and depth in angstroms or nanometers from the other antigen binders on the network of polynucleotides, which defines the predetermined inter-binder distances of the uniquely addressable loci of the antigen binders; and
the predetermined inter-binder distances of the uniquely addressable loci of the antigen binders match the intermolecular spacing of the antigens such that the antigen binders on the network of polynucleotides align spatially with the antigens on the surface of the target analyte.

16. The artificial biopolymer complex of claim 11, wherein:
the antigens form antigen clusters on the target analyte, wherein at least some of the antigen binders are arranged in antigen binder clusters; and
each binder of an antigen binder cluster is attached to uniquely addressable loci on the structural chain via the connector chain, wherein the uniquely addressable loci are separated by predetermined intra-cluster binder distances such that each antigen binder cluster are positioned on the network of polynucleotides in a predetermined two-dimensional or three-dimensional spatial pattern that matches a two-dimensional or three-dimensional spatial pattern of the antigens in an antigen cluster on the target analyte.

17. The artificial biopolymer complex of claim 16, wherein the binders of different clusters of antigen binders are separated by a predetermined inter-cluster distance such that antigen binder clusters are positioned on the network of polynucleotides in a predetermined two-dimensional or three-dimensional spatial pattern that matches a two-dimensional or three-dimensional spatial pattern of the antigen clusters.

18. The artificial biopolymer complex of claim 16, wherein:
each antigen binder in an antigen binder cluster is (i) a length and width in angstroms or nanometers from other binders of the antigen binder cluster on the network of polynucleotides or (ii) a length, width, and depth in angstroms or nanometers from the other binders of each antigen binder cluster on the network of polynucleotides, which defines the predetermined intra-cluster binder distances of the uniquely addressable loci of the binders in each antigen binder cluster; and
the predetermined inter-binder distances of the uniquely addressable loci of the binders of each antigen binder cluster match intermolecular spacing of the antigens in each cluster such that the binders in the antigen binder cluster on the network of polynucleotides align spatially with the antigens in an antigen cluster.

19. The artificial biopolymer complex of claim 16, wherein each of the predetermined intra-cluster binder distances of the uniquely addressable loci of each antigen binder cluster is between 1 nm and 15 nm.

20. The artificial biopolymer complex of claim 11, wherein the network of polynucleotides has a curvature such that the antigen binders are at least partially enveloped by a surface of the network of polynucleotides.

21. The artificial biopolymer complex of claim 11, wherein each of the antigens comprises one or more epitopes or domains;
the first set of binders are arranged in sets of clustered antigen binders;
each binder of a set of clustered antigen binders is attached to one of the two or more arms that form a junction; and
the binders of each of the sets of clustered antigen binders are attached to the arms at uniquely addressable loci that are a predetermined distance from the junction, wherein the uniquely addressable loci are separated by predetermined intra-binder distances such that each set of clustered antigen binders are positioned on the network of polynucleotides in a predetermined two-dimensional or three-dimensional spatial pattern that matches a two-dimensional or three-dimensional spatial pattern of the one or more epitopes on an antigen.

22. (canceled)

23. (canceled)

24. (canceled)

25. The artificial biopolymer complex of claim 11, wherein the network of polynucleotides has a second surface,
wherein the first surface is attached with the antigen binders, and the second surface is attached with biomarker binders,
wherein the biomarker binders bind to biomarkers on immune cells,
wherein the uniquely addressable loci at which the biomarker binders are attached are separated by predetermined inter-binder distances such that biomarker binders are positioned on the network of polynucleotides in a predetermined two-dimensional or three-dimensional spatial pattern that matches a two-dimensional or three-dimensional spatial pattern of the biomarkers on the immune cells.

26.-36. (canceled)

37. A method for treating a subject, the method comprising:
obtaining the artificial biopolymer complex of claim 1; and
administering the artificial biopolymer complex to the subject in an amount sufficient to provide a treatment effect.

38.-41. (canceled)
